(19) United States
(12) Patent Application Publication
(10) Pub. No.: US 2025/0334588 A1
(43) Pub. Date: Oct. 30, 2025

(54) TREATING TUBEROUS SCLEROSIS COMPLEX-ASSOCIATED DISEASES

(71) Applicant: The Brigham and Women's Hospital, Inc., Boston, MA (US)

(72) Inventors: Yan Tang, Boston, MA (US); Elizabeth P. Henske, Cambridge, MA (US); David Joseph Kwiatowski, Weston, MA (US)

(21) Appl. No.: 18/855,136

(22) PCT Filed: Apr. 10, 2023
(86) PCT No.: PCT/US2023/018041

§ 371 Date: Oct. 8, 2024

Related U.S. Application Data
(60) Provisional application No. 63/329,416, filed on Apr. 9, 2022.

(51) Int. Cl. G01N 33/68; A61K 45/06; C12N 15/113

(52) U.S. Cl. CPC G01N 33/6872; A61K 45/06; C12N 15/1136; G01N 33/6893; C12N 2310/11; C12N 2310/14; C12N 2310/531; G01N 2333/475; G01N 2800/52

(57) ABSTRACT

Provided herein are compositions and methods using a therapeutic agent targeting mTORCI and a therapeutic agent targeting MDK for treating a Tuberous Sclerosis Complex (TSC)-associated disease, e.g., Angiomyolipoma (AML) and lymphangioleiomyomatosis (LAM), or for treating sporadic LAM/AML. Also provided are methods of identifying subjects for treatment, e.g., with checkpoint inhibitors.

### CLAIM OF PRIORITY

This application claims the benefit of U.S. Provisional Application Ser. No. 63/329,416, filed on Apr. 9, 2022. The entire contents of the foregoing are incorporated herein by reference.

### FEDERALLY SPONSORED RESEARCH OR DEVELOPMENT

This invention was made with Government support under Grant No. W81XWH-19-1-0152 from the Department of Defense, and Grant No. HL131022 awarded by the National Institutes of Health. The Government has certain rights in the invention.

### TECHNICAL FIELD

Provided herein are compositions and methods using a therapeutic agent targeting mTORC1 and a therapeutic agent targeting MDK for treating a Tuberous Sclerosis Complex (TSC)-associated disease, e.g., Angiomyolipoma (AML) and lymphangioleiomyomatosis (LAM), or for treating sporadic LAM/AML. Also provided are methods of identifying subjects for treatment, e.g., with checkpoint inhibitors.

### BACKGROUND

Tuberous Sclerosis Complex (TSC) is an autosomal dominant disease with an incidence of 1:6000 births. TSC is caused by loss-of-function mutations in the tumor suppressor genes TSC1 and TSC23. Second hit loss of the remaining wild-type copy of TSC1 or TSC2 leads to hyperactive mammalian target of rapamycin complex 1 (mTORC1), and drives tumor growth in multiple organs³. Angiomyolipoma (AML) and lymphangioleiomyomatosis (LAM) are common and related manifestations of TSC that can lead to renal and pulmonary insufficiency, respectively^{4,5}. AML and LAM also occur sporadically in patients without TSC³⁻⁵.

### SUMMARY

Provided herein are methods for treating a Tuberous Sclerosis Complex (TSC)-associated disease. The methods comprise administering to a subject in need thereof a therapeutically effective amount of a therapeutic agent targeting mTORC1 and a therapeutic agent targeting MDK. Additionally provided are therapeutic agents targeting mTORC1 and a therapeutic agent targeting MDK for use in a method of treating a Tuberous Sclerosis Complex (TSC)-associated disease. In some embodiments, the TSC-associated disease is angiomyolipoma (AML) or lymphangioleiomyomatosis (LAM). In some embodiments, the TSC-associated disease is a cortical dysplasia, subependymal nodule, subependymal giant cell astrocytoma (SEGA), cardiac rhabdomyoma, dermatologic or ophthalmic tumor, renal cyst, multifocal micronodular pneumocyte hyperplasia (MMPH), splenic hamartoma, or perivascular epithelioid tumor (PEComa).

Also provided herein are methods for treating lymphangioleiomyomatosis (LAM) or angiomyolipoma (AML). The methods comprise administering to a subject in need thereof a combination of a therapeutic agent targeting mTORC1 and a therapeutic agent targeting MDK. Additionally provided are a therapeutic agent targeting mTORC1 and a therapeutic agent targeting MDK for use in a method of treating lymphangioleiomyomatosis (LAM) or angiomyolipoma (AML) in a subject. In some embodiments, the subject does not have a diagnosis of TSC or a mutation in the TSC1 or TSC2 tumor suppressor genes.

Also provided herein are methods for selecting a treatment for a Tuberous Sclerosis Complex (TSC)-associated disease in a subject. The methods comprise determining a level of MDK in a sample from the subject; comparing the level of MDK in the sample to a reference level of MDK; identifying a subject who has a level of MDK above the reference level; and selecting a treatment comprising administering to identified the subject a therapeutically effective amount of a therapeutic agent targeting mTORC1 and a checkpoint inhibitor, and optionally a therapeutic agent targeting MDK. In some embodiments, the TSC-associated disease is angiomyolipoma (AML) or lymphangioleiomyomatosis (LAM). In some embodiments, the TSC-associated disease is a cortical dysplasia, subependymal nodule, subependymal giant cell astrocytoma (SEGA), cardiac rhabdomyoma, dermatologic or ophthalmic tumor, renal cyst, multifocal micronodular pneumocyte hyperplasia (MMPH), splenic hamartoma, or perivascular epithelioid tumor (PEComa).

Further, provided herein are methods for selecting a treatment for lymphangioleiomyomatosis (LAM) or angiomyolipoma (AML). The methods comprise: determining a level of MDK in a sample from the subject; comparing the level of MDK in the sample to a reference level of MDK; identifying a subject who has a level of MDK above the reference level; and selecting a treatment comprising administering to identified the subject a therapeutically effective amount of a therapeutic agent targeting mTORC1 and a checkpoint inhibitor, and optionally a therapeutic agent targeting MDK. In some embodiments, the subject does not have a diagnosis of TSC or a mutation in the TSC1 or TSC2 tumor suppressor genes. In some embodiments, the methods further comprise administering the treatment to the identified subject.

Also provided herein are compositions comprising a combination of a therapeutic agent targeting mTORC1 and a therapeutic agent targeting MDK, and optionally a checkpoint inhibitor.

In some embodiments, the mTORC1 inhibitor is selected from the group consisting of MLN0128, MHY1485, PI-103, PP242 (torkinib), PP30, XL388, AZD2014 (vistusertib), voxtalisib (SAR24540; XL765), vistusertib, OSI-227, WAY-600, WYE-132, WYE-687, or sapanisertib (TAK-228); PF-04691502; Gedatolisib (PKI-587; PF05212384); AZD 8055 ((5-(2,4-bis((S)-3-methylmorpholino)pyrido[2,3-d]pyrimidin-7-yl)-2-methoxyphenyl) methanol); Torin-1 (1-[4-[4-(1-oxopropyl)-1-piperazinyl]-3-(trifluoromethyl)phenyl]-9-(3-quinolinyl)-benzo[h]-1,6-naphthyridin-2 (1H)-one); torin-2; apitolisib; gedatolisib; GSK2126458 (GSK458); CC-223; 4H-1-benzopyran-4-one derivatives; rapamycin (sirolimus) and derivatives thereof, including: temsirolimus, umirolimus, everolimus, ridaforolimus (deforolimus), and zotarolimus; rapalogs, optionally AP23464, AP23841, 40-(2-hydroxyethyl) rapamycin; 40-[3-hydroxy (hydroxymethyl)methylpropanoate]-rapamycin (CC1779); 40-epi-(tetrazolyt)-rapamycin (ABT578); 32-deoxorapamycin; 16-pentynyloxy-32 (S)-dihydrorapanycin; and phosphorus-containing rapamycin derivatives; cornarin A, dactolisib, omipalisib, samotolisib, KU-0063794, gadatolisib, dactosulib tosylate, CC-115, apitolisib, bimarilisib, VS-5584, GDC-0349, CZ415, WYE-354, onatasertib, mTOR-inhibitor 3, palomid 529, PQR620, (+)-usnic acid, MT 63-78, MTI-31, FT-1518, AZD3147, and RMC-5552.

In some embodiments, the MDK inhibitor is iMDK; an anti-midkine antibody; an RNA Aptamer; or an inhibitory nucleic acid targeting midkine. In some embodiments, the inhibitory nucleic acid targeting midkine is an antisense oligonucleotide, siRNA, or shRNA.

In some embodiments, the methods further comprise administering a checkpoint inhibitor or a treatment comprising chemotherapy, radiotherapy, and/or resection.

In some embodiments, the checkpoint inhibitor is, e.g., an inhibitor of PD-1 signaling, optionally an antibody that binds to PD-1, CD40, or PD-L1; an inhibitor of Tim3 or Lag3, optionally an antibody that binds to Tim3 or Lag3; an inhibitor of CTLA4, optionally an antibody that binds to CTLA-4; or an inhibitor of T-cell immunoglobulin and ITIM domains (TIGIT), optionally an antibody that binds to TIGIT.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

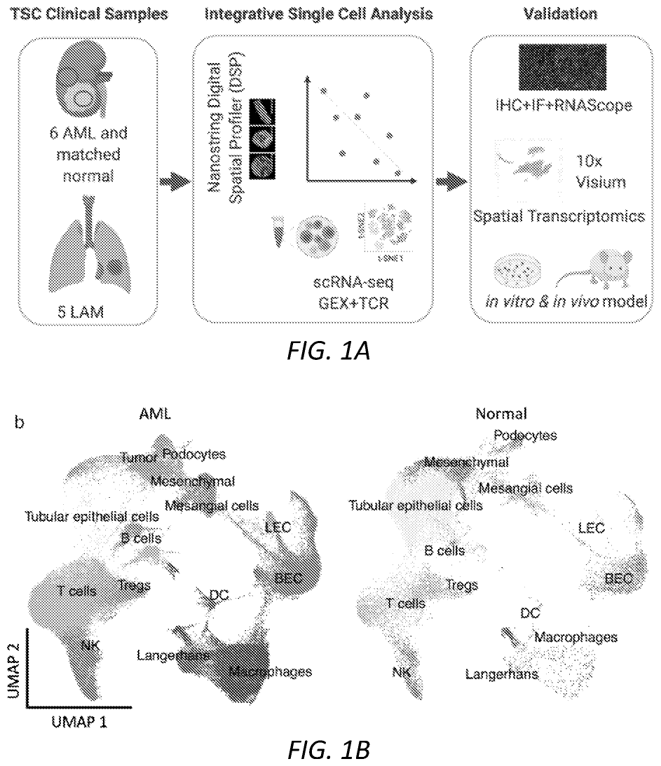

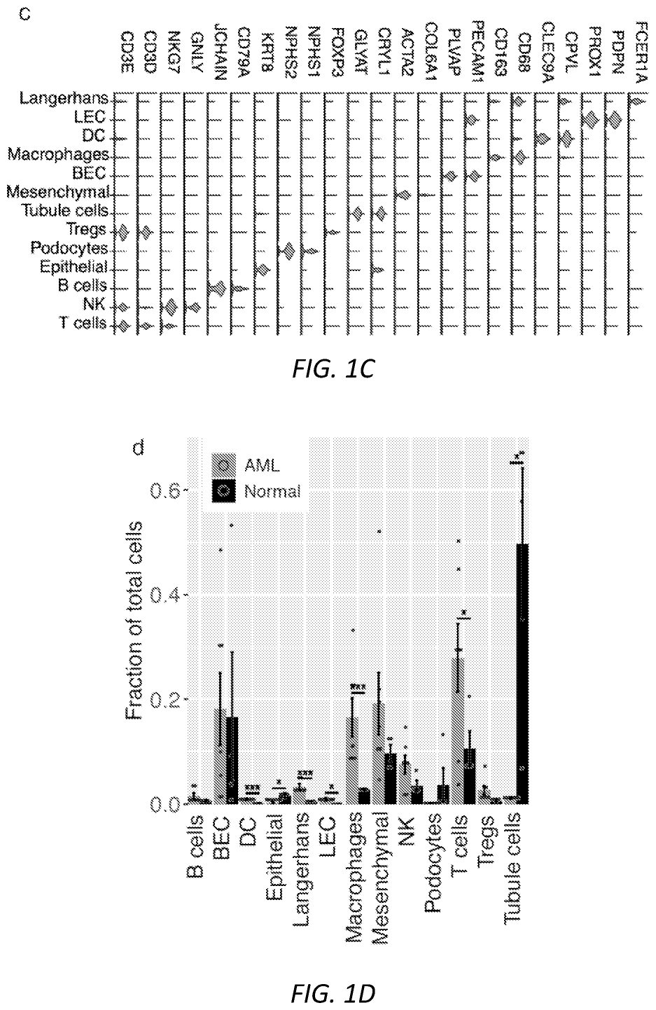

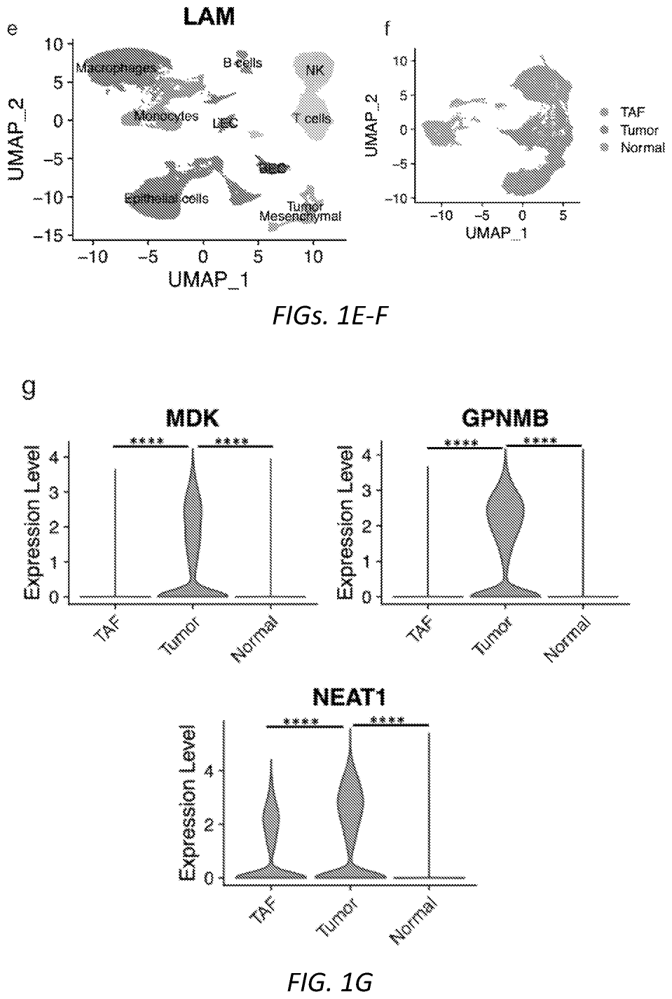

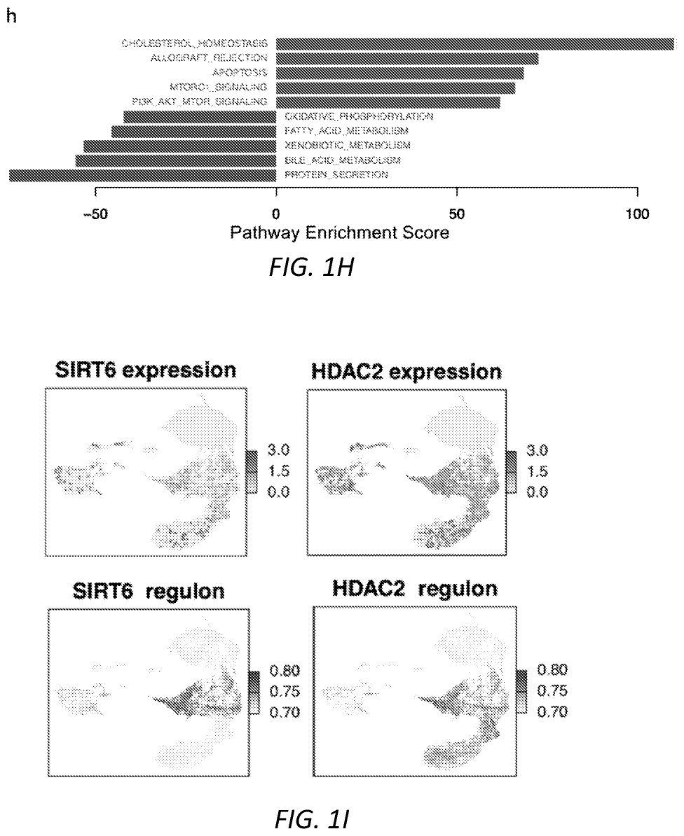

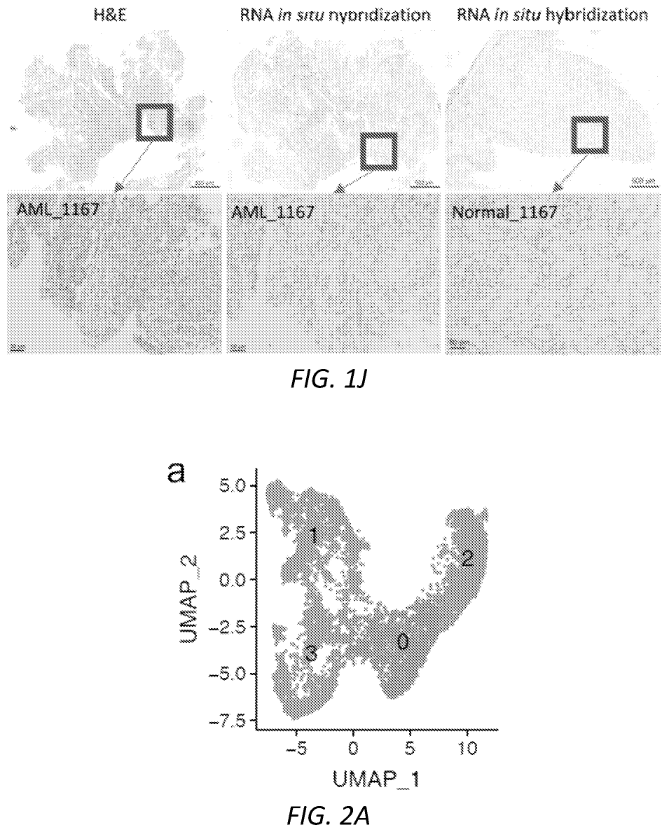

FIGS. 1A-J. Single-cell atlas of angiomyolipoma (AML) and lymphangioleiomyomatosis (LAM).
a. Workflow showing samples collected and integrative analysis of scRNA-Seq, paired scTCR-Seq and spatial transcriptomics, followed by in vitro and in vivo mechanistic studies (created with BioRender.com).
b. Uniform Manifold Approximation and Projection (UMAP) plots of major cell types identified in six AML tumors (left) and four matched normal kidneys (right). LEC: lymphatic endothelial cells; BEC: blood endothelial cells; Tregs: regulatory T cells; NK: natural killer cells; DC: dendritic cells.
c. Violin plots of marker genes of each cell type. The y axis represents the normalized gene expression value.
d. Quantification of fractional representation of cell types in tumors (n=6) and matched normal (n=4) tissues. Standard errors are shown for each group. *p<0.05, **p<0.01, ***p<0.001, two-sided t-test. Data are presented as mean±SEM.
e. UMAP plot showing major cell types identified in five LAM lungs. LEC: lymphatic endothelial cells; BEC: blood endothelial cells; NK: natural killer cells.
f. Re-clustering of mesenchymal cells from AML tumors and matched normal kidneys.
g. Expression of midkine (MDK), GPNMB, and NEAT1 in AML tumor cells compared to normal kidney mesenchymal cells and TAFs. The y axis represents the normalized gene expression value. ****p<0.001 (Wilcoxon test).
h. Hallmark pathways enriched in AML cells and in matched normal mesenchymal cells. x-axis shows pathway enrichment score. Top five enriched pathways are shown.
i. Expression and regulon activity of SIRT6 and HDAC2 in tumor and normal mesenchymal cells using same UMAP coordinates of F. Regulon activities of these two transcription factors were calculated based on the expression of their target genes. Note: TAF cells colored in grey were not analyzed for regulon activity.
j. RNA in situ hybridization (ISH) assessment of MDK in AML tumor and in adjacent normal kidney. RNA in situ hybridization assessments were performed on the same samples subjected to single cell analysis. An H&E image of the same sample is also provided. These are representative images of 5 tumor samples and 4 matched normal samples.

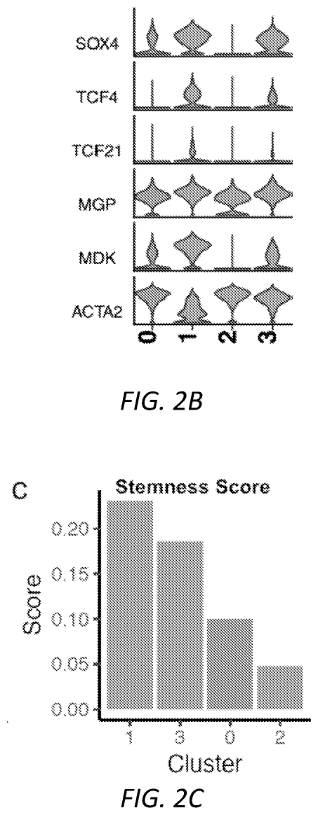

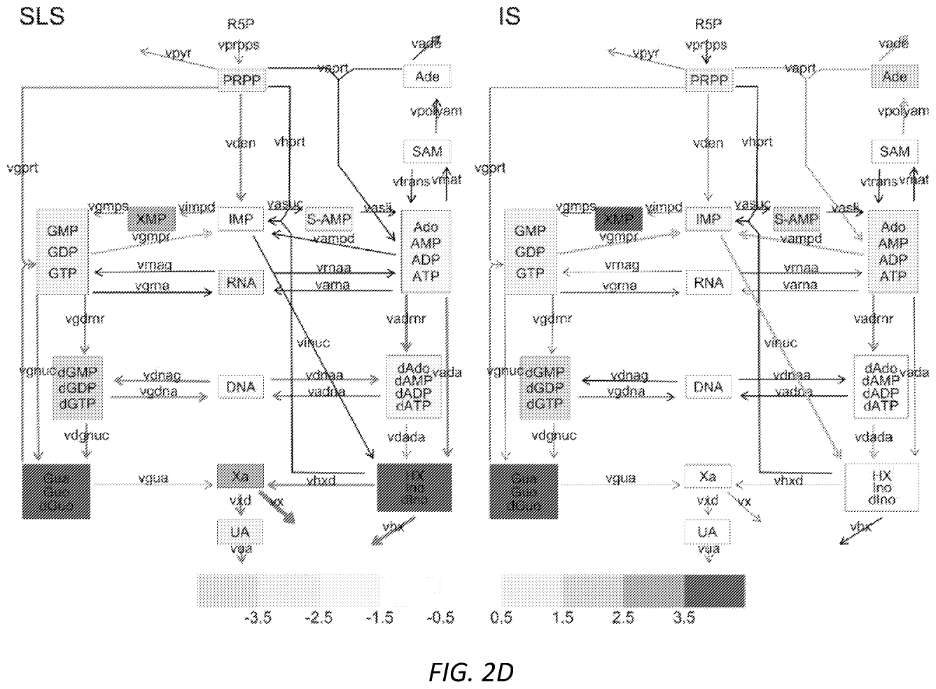

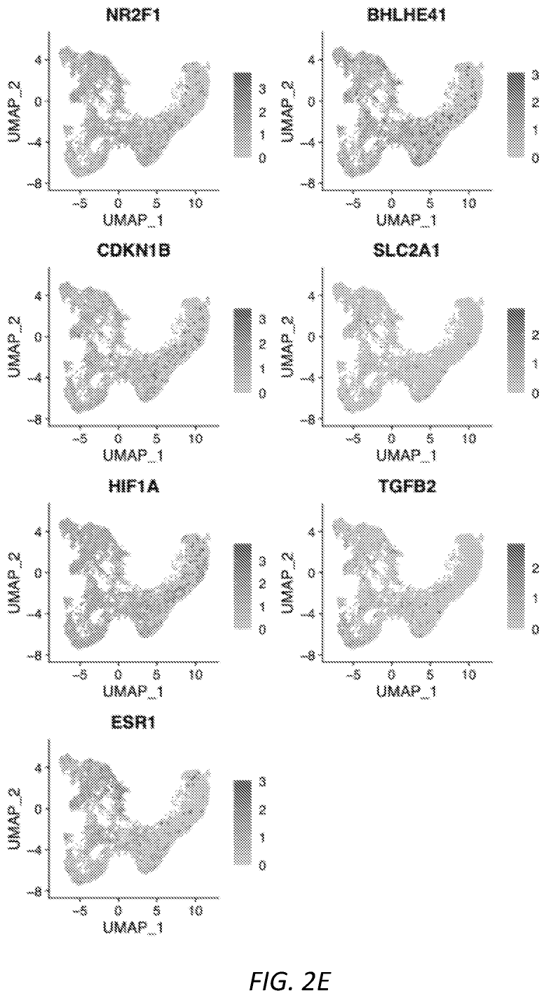

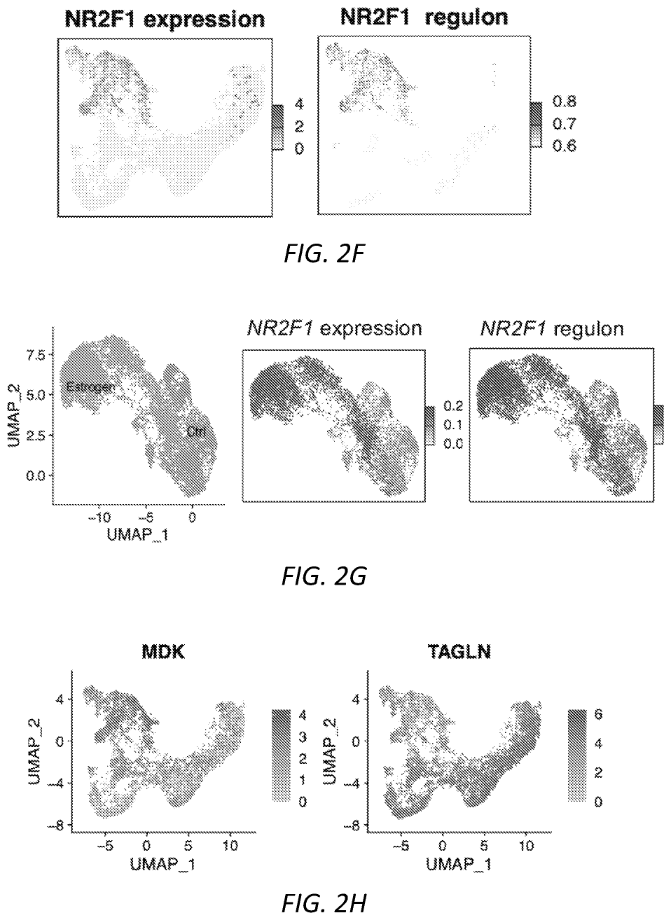

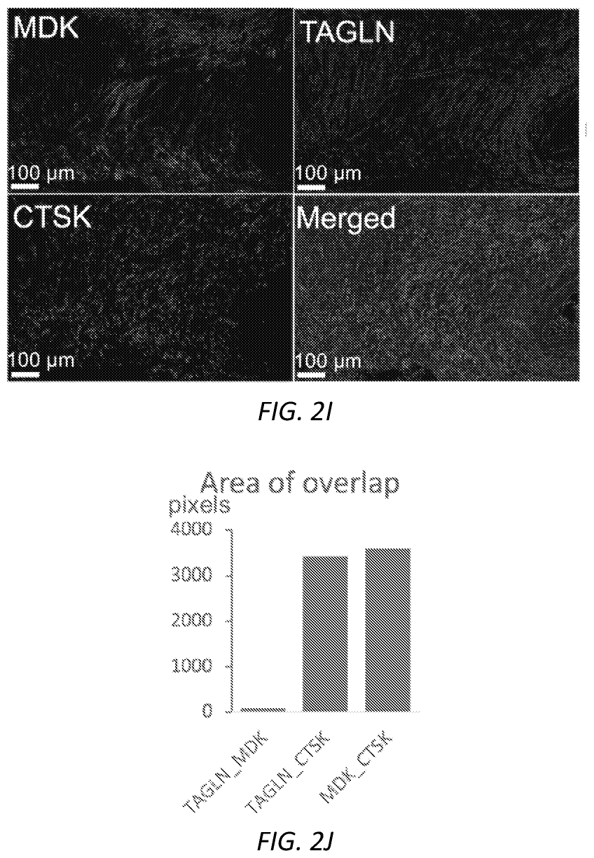

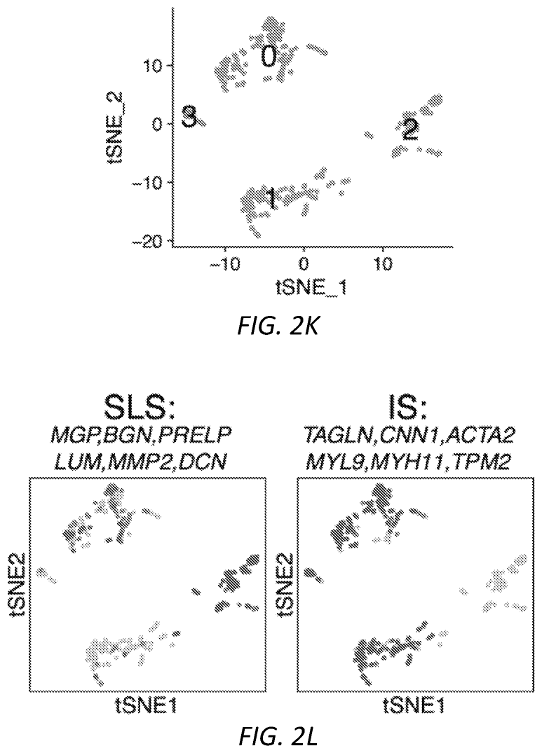

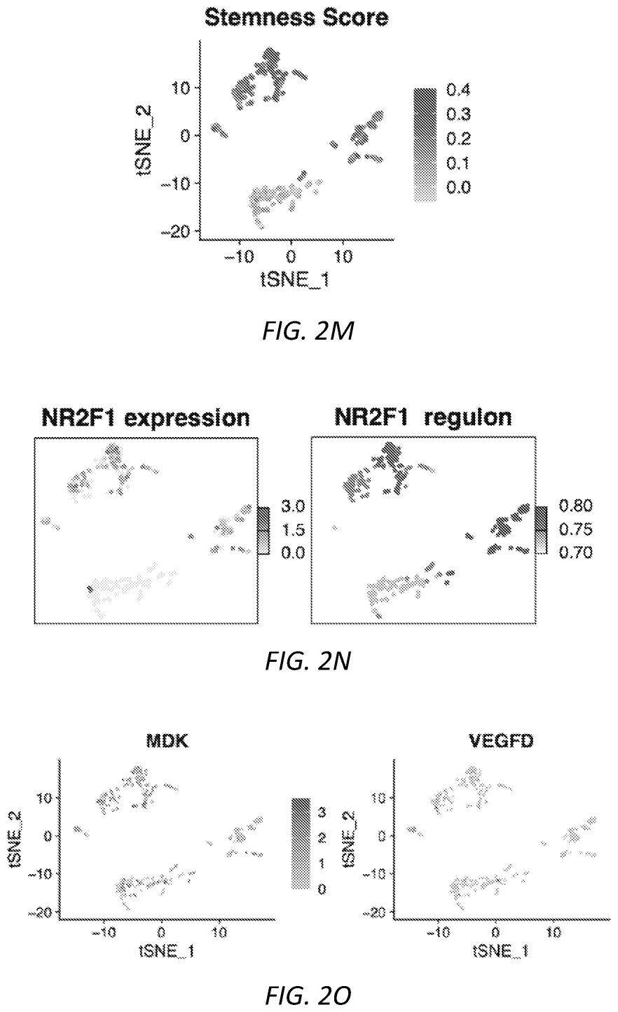

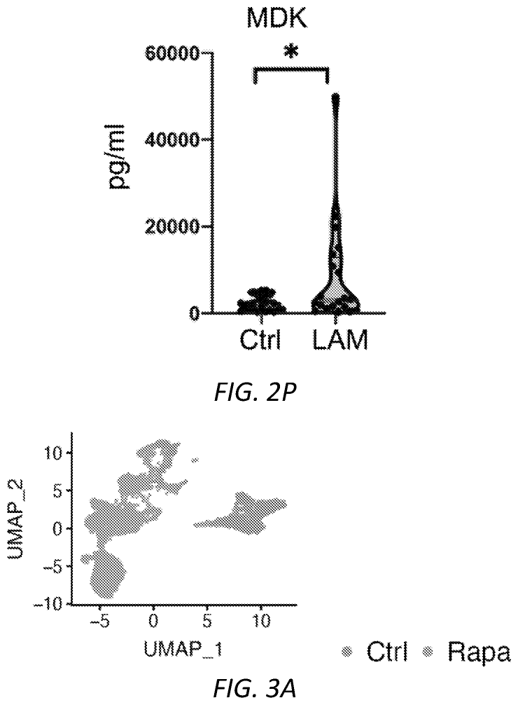

FIGS. 2A-P Heterogeneous cellular states in AML and LAM.
a. UMAP plot of AML cells only showing two distinct clusters (cluster 1 and cluster 2) and two transitional clusters (0 and 3).
b. Violin plots of highly expressed genes of each cluster. The y axis represents the normalized gene expression value.
c. Stemness score calculated using 50 tumor stem cell marker genes for each cluster.
d. Inferred purine metabolism flux in SLS and IS populations relative to matched normal mesenchymal cells using pseudo-bulk RNA-seq generated from single cell transcriptomes. Relative levels of hypoxanthine (HX) and inosine/deoxyinosine (Ino/dlno) are upregulated in SLS.
e. Feature plots of expression of dormancy marker genes in the tumor cell population.
f. Analysis of NR2F1 expression and regulon activity in AML cells. Left panel: expression of NR2F1 in cluster 1 and 2; right panel: NR2F1 regulon activity based on 41 downstream target genes. Note: only cluster 1 and cluster 2 were compared for regulon activity. Other clusters are colored in grey.
g. NR2F1 expression and regulon activity in LAM patient-derived TSC2-deficient cells 621-101 cells with and without estrogen treatment. Left panel: t-SNE plot of cells from estradiol treated group and cells from control group; middle panel: expression of NR2F1; right panel: NR2F1 regulon activity.
h. Expression of MDK and TAGLN in AML cell clusters.
i. Triple staining for MDK, TAGLN, and CTSK. Representative images of 5 samples.
j. Quantification of co-staining of MDK, TAGLN and CTSK shows little co-localization of MDK and TAGLN (first bar), while both TAGLN and MDK co-localize with the tumor marker gene CTSK (second and third bars). The y axis represents area of overlap (arbitrary unit).
k. Re-clustering of the LAM cells from 5 LAM lungs, revealing four clusters.
l. Average expression of SLS (left) and IS (right) marker genes in the LAM clusters. An SLS population (cluster 2 in K), IS population (cluster 1 in K), and an intermediate state (Cluster 0 and 3 in K) were identified.
m. SLS population (Cluster 2) and intermediate state LAM cells (Cluster 0 and 3) show high stemness scores.
n. Expression and regulon analysis of NR2F1 in LAM cells. Left panel: NR2F1 expression; right panel: NR2F1 regulon activity corresponding to the degree of regulation of 23 downstream target genes.
o. Feature plot showing expression of MDK and VEGFD in LAM cells.
p. ELISA assessment of serum MDK levels in healthy cohorts (n=19) and in LAM patients (n=20). *: pvalue=0.0361, two-sided t-test.

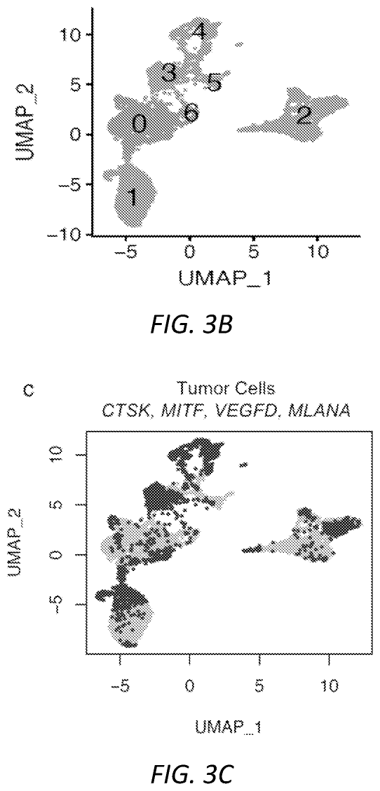

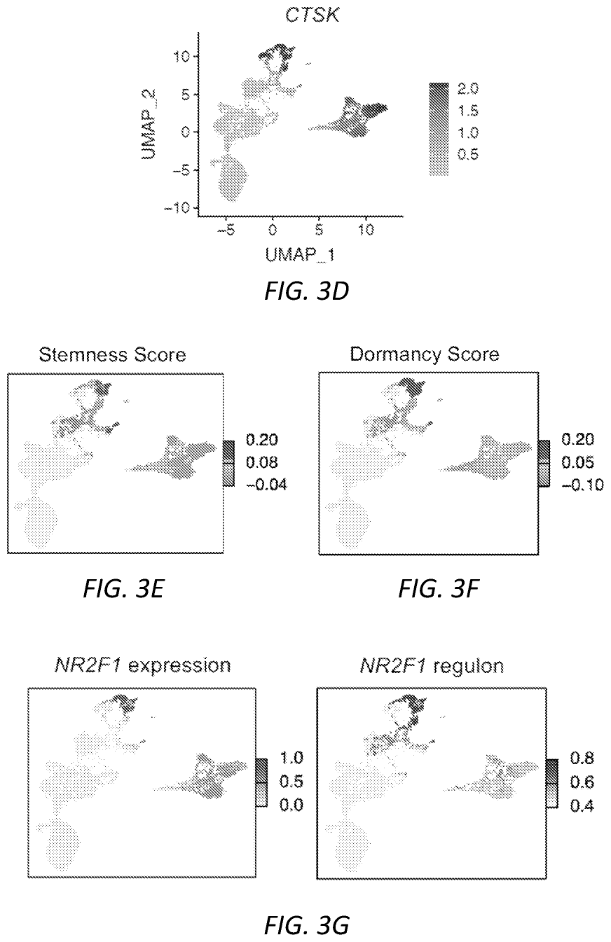

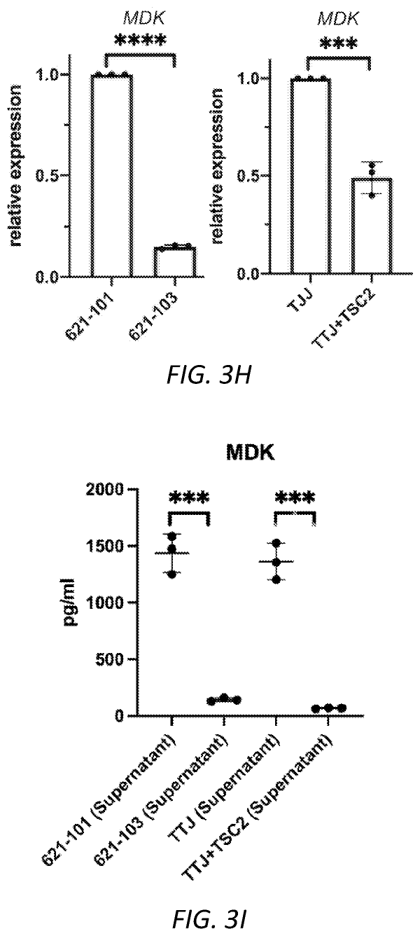

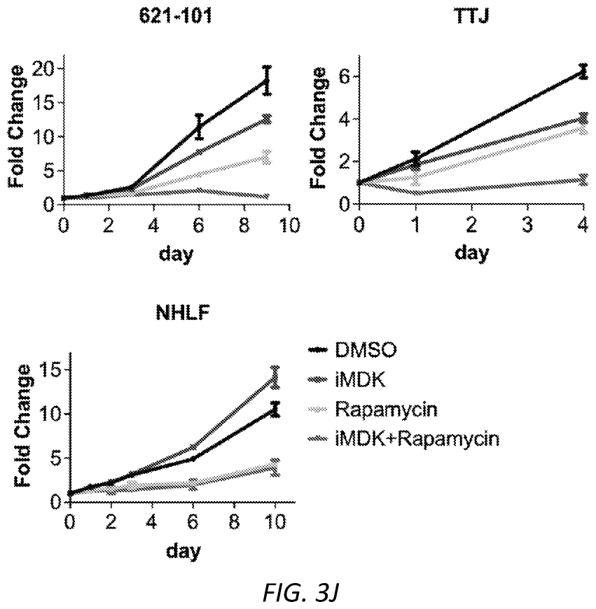

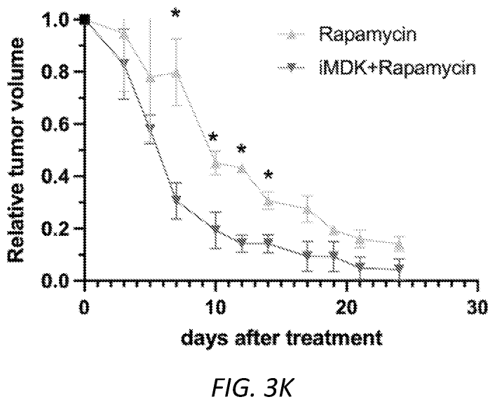

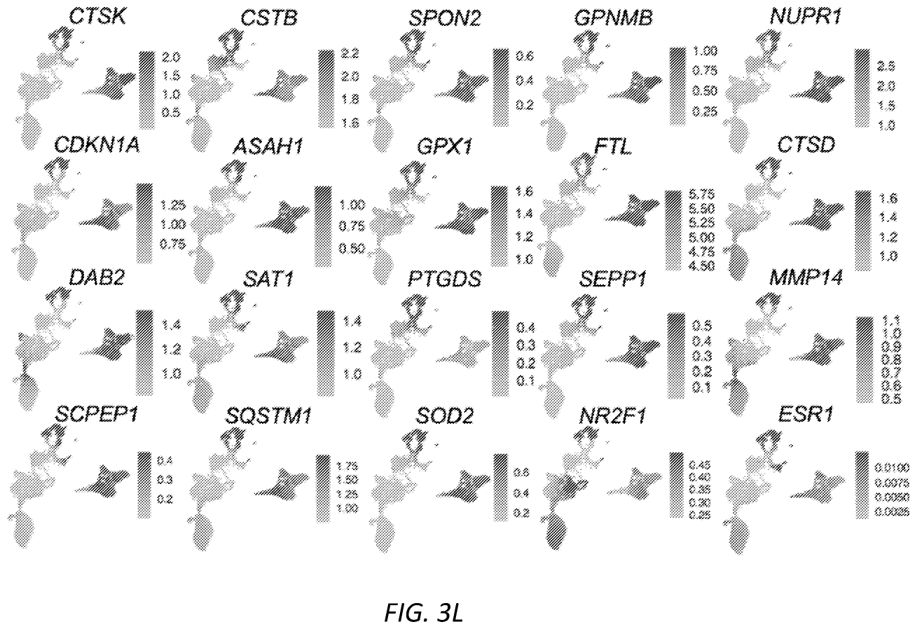

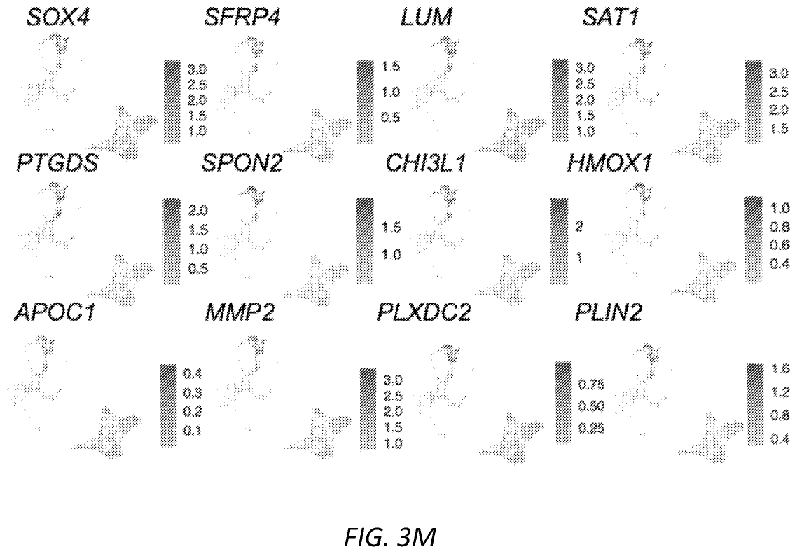

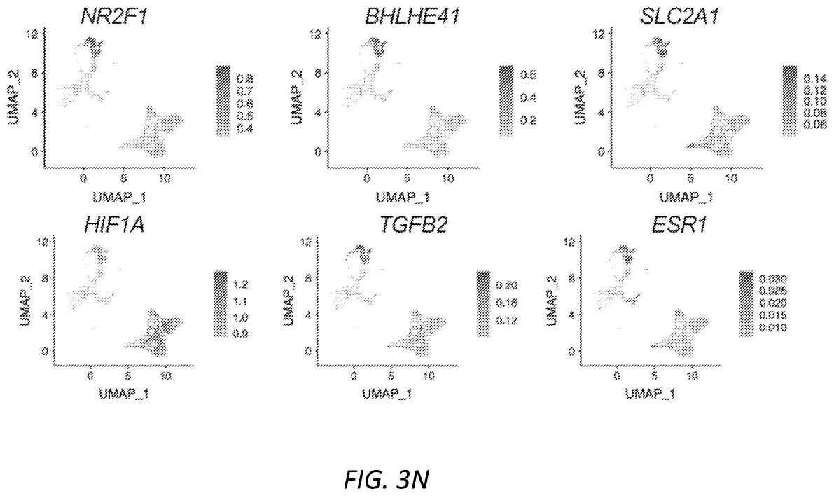

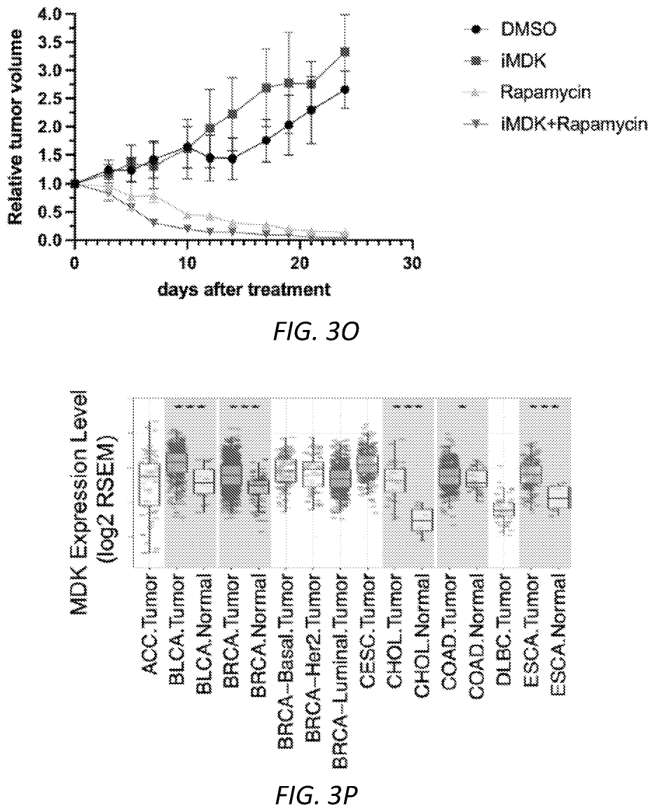

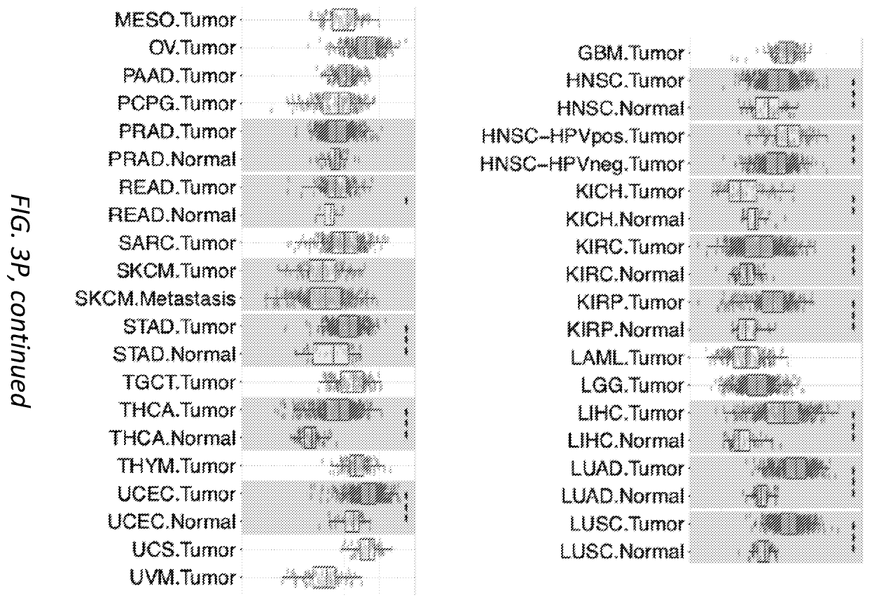

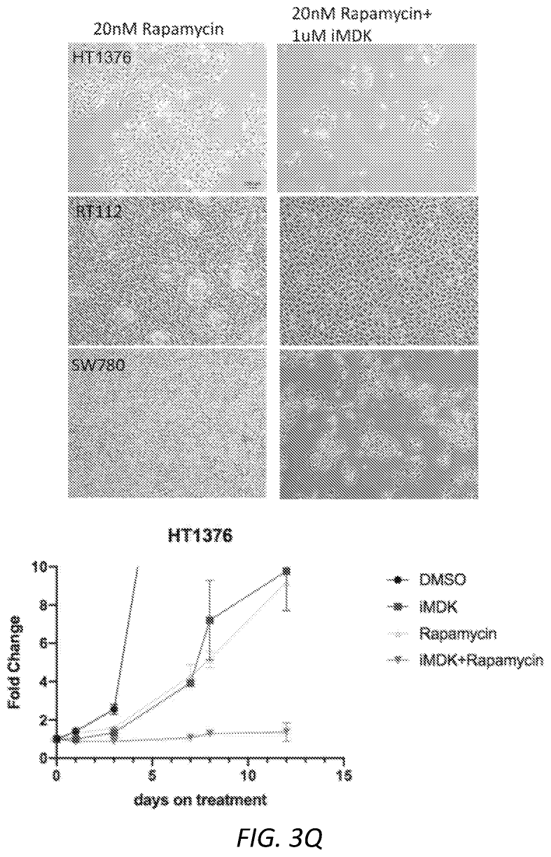

FIGS. 3A-Q. The stem-like population of AML cells may contribute to rapamycin resistance.
a. UMAP plot of primary cultures derived from AML tumor colored by treatment. Cells were treated with DMSO as control or 20 nM rapamycin for 24 hours before scRNA-Seq.
b. UMAP plot of the AML-derived primary cell culture.
c. Expression of 5 AML markers in the primary AML culture before and after rapamycin treatment (as in A).
d. CTSK expression in cells before and after rapamycin (as in A).
e. Cluster 4 in the DMSO control group showed high stemness score, calculated using a panel of 50 cancer stem cell marker genes (see method). Note that stemness score was only calculated in DMSO control group, and the cells in the rapamycin treatment group are colored in light grey.
f. Cluster 4 in the DMSO control group showed high dormancy score, calculated using known dormancy marker genes (see method). Note that dormancy score was only calculated in DMSO control group, and the cells in rapamycin treatment group are colored in light grey.
g. Expression (left) and regulon activity (right) of NR2F1 in control group. Note: only cluster 2 and cluster 4 were comparatively analyzed for regulon activity. The color bars indicate expression level and regulon activity only for these two clusters; other cells are colored in light grey.
h. Relative expression of MDK in TSC2-deficient cell lines compared to TSC2-addback cells. Left panel: patient-derived TSC2-deficient 621-101 cells (n=3) compared to TSC2-addback 621-103 cells (n=3); right panel: mouse kidney derived TSC2-deficient TTJ cells (n=3) compared to TSC2-addback cells (n=3). Data are presented as mean±SD. ***p-value=0.0004; ****p-value<0.0001, two-sided t-test.
i. MDK protein level in the cell culture supernatants (n=3 per cell line) measured by ELISA. Data are presented as mean±SD. ***p<0.001, two-sided t-test.
j. Proliferation measured by crystal violet assay. Treatments were: DMSO, 1 μM iMDK, 20 nM rapamycin, and combination of iMDK and rapamycin for days indicated. All experiments were replicated 3 times. 621-101 and TTJ are TSC-deficient. NHLF: normal human lung fibroblasts. Data are presented as mean±SD.
k. Tumor size reduction relative to pre-treatment tumor volume in rapmayin treatment and combined iMDK and rapamycin treatment groups. p-values were calculated by two-sided t-test. *: p<0.05. Data are presented as mean+SD. Day 7: p-value=0.001; day 10: p-value=0.001; day 12: p-value=0.001; day 14: p-value=0.001. Relative tumor size after treatment for all treatment groups can be found in FIG. 30: TTJ xenograft mice (n=6 per group) were treated 3 times/wk with DMSO, iMDK (9 mg/kg), rapamycin (3 mg/kg), or combined iMDK (9 mg/kg) and rapamycin (3 mg/kg). Averaged tumor size was reduced to <20% of pre-treatment volume after 4 treatments in the combination treatment group, in contrast to 8 treatments in the rapamycin treatment group.
l. Expression of genes identified as upregulated in AML cells in this study before and after rapamycin treatment in the primary culture.
m. Expression of genes upregulated in SLS in the control group.
n. Expression of dormancy marker genes in the control group.
o. Tumor volume relative to pre-treatment tumor volume. Data are presented as mean±SD. Tumor volume was measured immediately before each treatment. TTJ xenograft mice (n=6 per group) were treated 3 times/wk with DMSO, iMDK (9 mg/kg), rapamycin (3 mg/kg), or combined iMDK (9 mg/kg) and rapamycin (3 mg/kg).
p. Expression of MDK across cancer types and matched normal tissues (data obtained from TCGA). Box plots show the first quartile, median, and the third quartile. P-value Significant Codes: 0≤***<0.001≤**<0.01≤*<0.05≤. <0.1. Refer number of RNA-seq datasets for each cancer type:
cancer.gov/aboutnci/organization/ccg/research/structural-genomics/tcga/studied-cancers.
q. Cell growth inhibited by combination treatment of rapamycin and iMDK on 3 bladder cancer cell lines. Left panel: microscopy pictures showing growth of 3 bladder cancer cell lines treated with 20 nM rapamycin alone (first column) or combination treatment of 20 nM rapamycin and 1 μM iMKD (second column) for 6 days. Scale bar, 100 μm (for all images). Right panel: cell proliferation, assessed by crystal violet assay, of bladder cell line HT1376 on the treatment of DMSO (vehicle), 20 nM rapamycin, 1 μM iMDK or combination of 20 nM rapamycin and 1 μM iMDK for 14 days. All experiments were replicated 3 times. Data are presented as mean±SD.

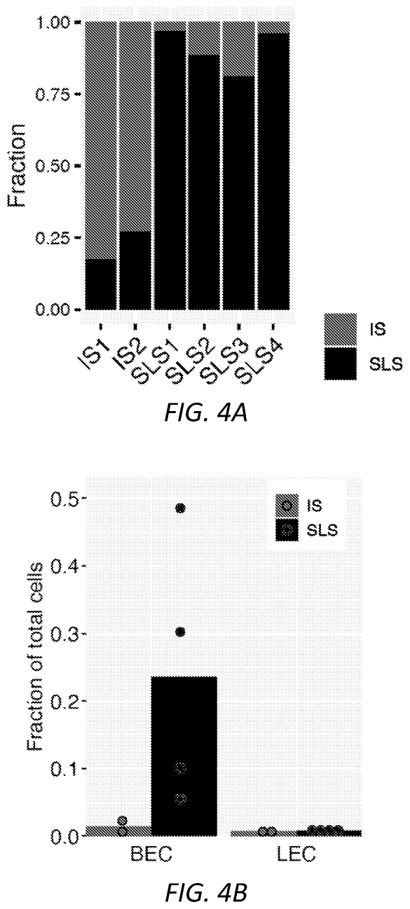

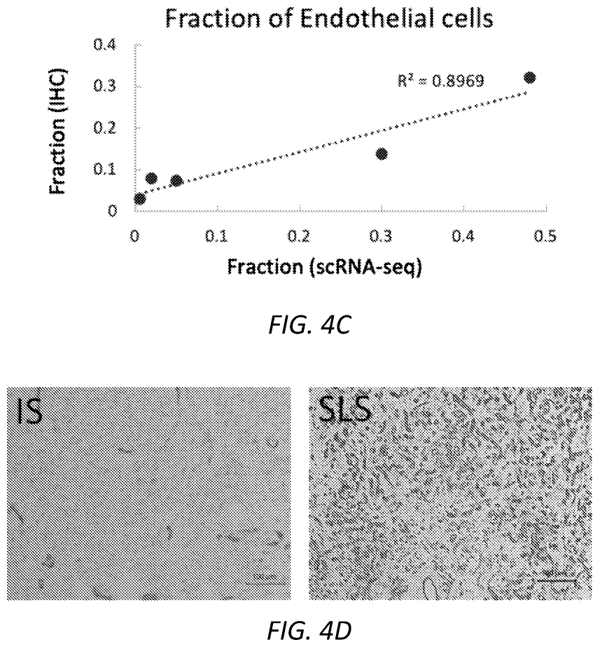

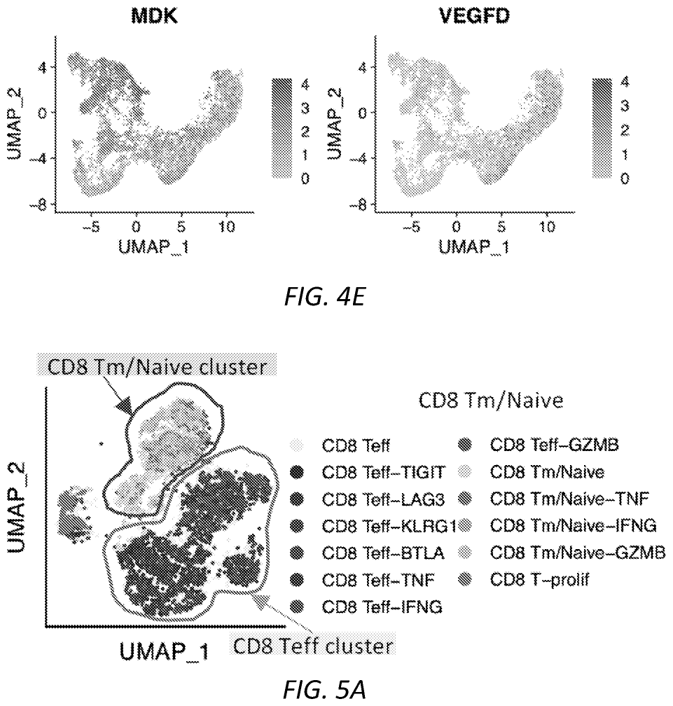

FIGS. 4A-E. Endothelial cell remodeling in SLS-dominant tumors.
a. Percentage of SLS and IS cells in the six AML tumors profiled.
b. Quantification of fractional representation of blood endothelial cells (BEC) and lymphatic endothelial cells (LEC) in SLS-dominant (n=4) and IS-dominant tumors (n=2).
c. Comparison of percentage of blood endothelial cells identified by single cell profiling and CD31 IHC for five AML tumors.
d. Representative IHC staining of endothelial cells with anti-CD31 in two IS-dominant tumors and in three SLS-dominant tumors.
e. Expression of MDK and VEGFD in AML cell populations. The MDK figure is also shown in FIG. 2h, and is repeated here for ease of comparison.

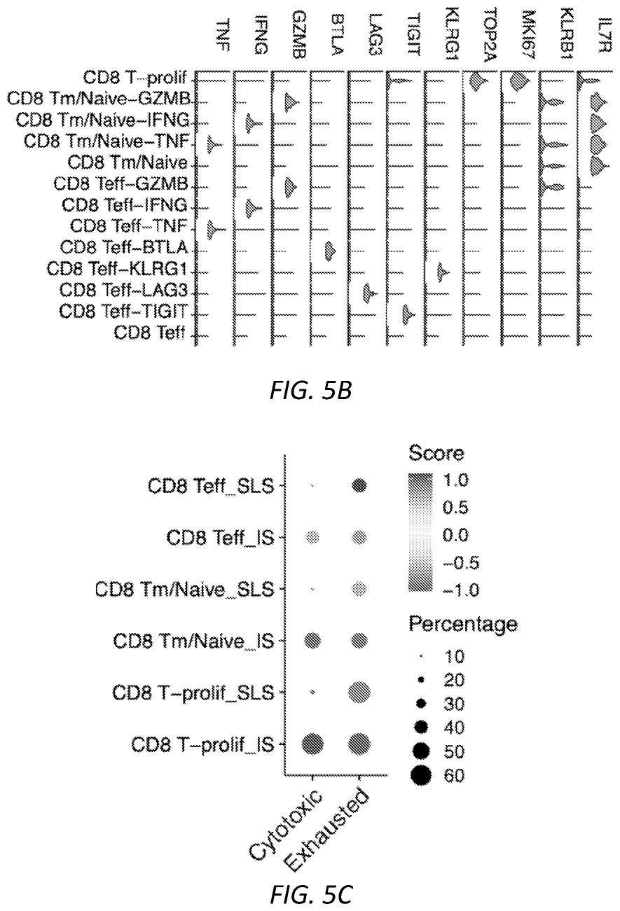

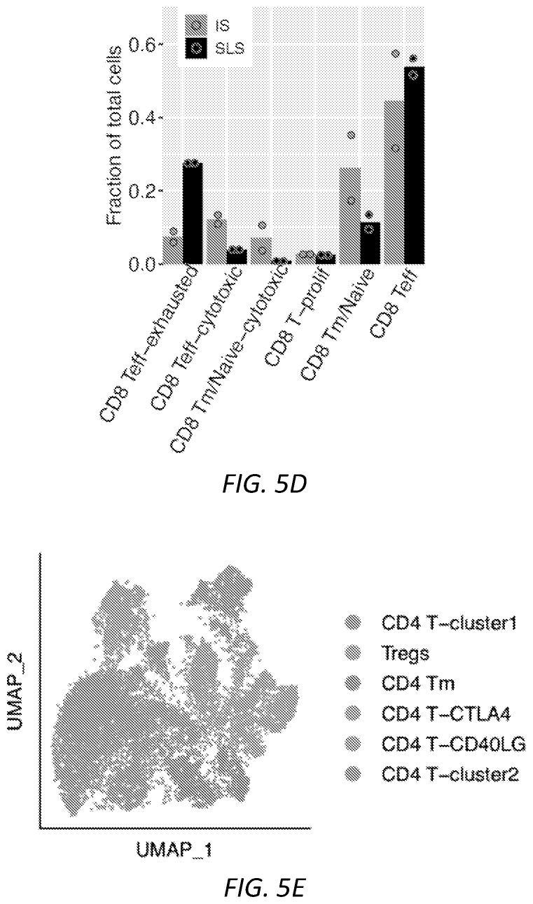

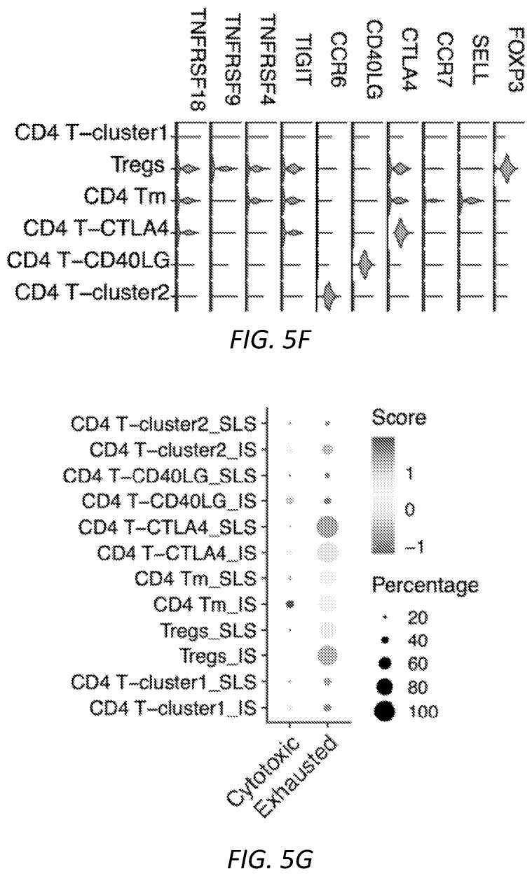

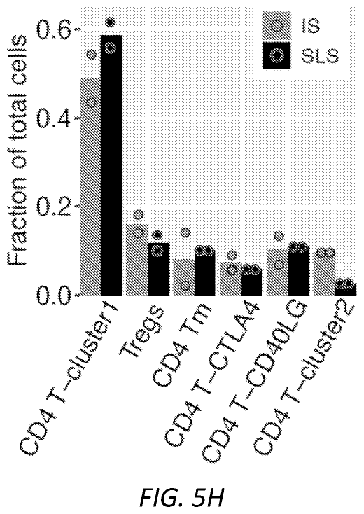

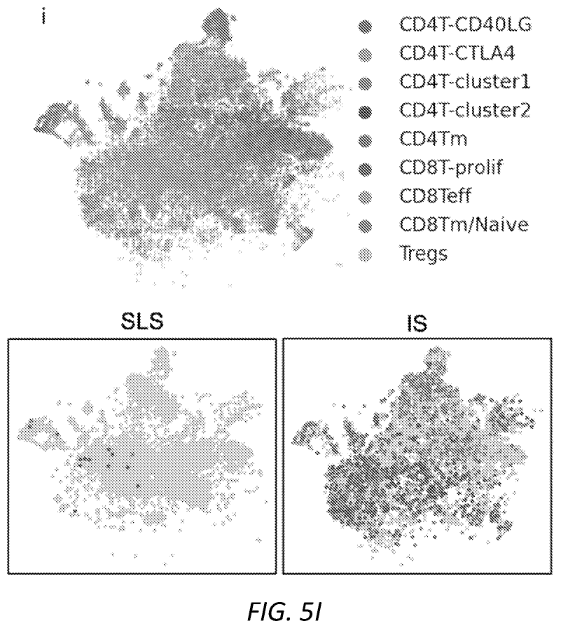

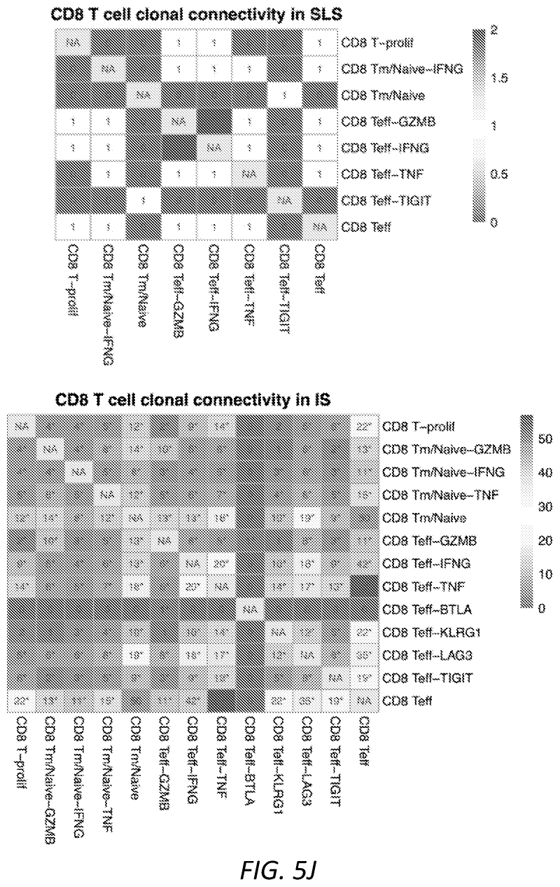

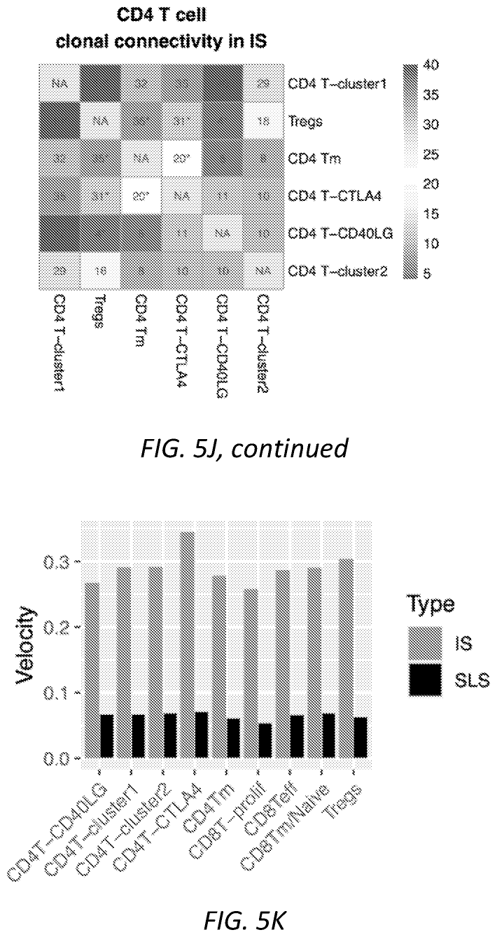

FIGS. 5A-K. T cell dysfunction and suppressed T cell clonal expansion in SLS-dominant tumors.
a. UMAP plot of CD8+ T cells obtained from four AML tumors (downsampled to have equal number of cells from SLS or IS dominant tumors). Phenotypic clusters are represented in distinct colors. CD8 Teff: effector CD8+ T cells; CD8 Tm/Naïve: memory/naïve CD8+ T cells; CD8 T-prolif: proliferating CD8+ T cells.
b. Violin plot of representative marker genes of each cluster of CD8+ T cells defined in (A). The y-axis represents the normalized gene expression values.
c. Module score of T cell exhaustion or cytotoxicity across major CD8+ T cell population in SLS or IS dominant tumors. Exhaustion module score was calculated based on relative expression of checkpoint genes TIGIT, LAG3, BTLA and KLRGI. Cytotoxicity module score was calculated based on relative expression of cytotoxic effector genes GZMB, IFNG and TNF. Module scores were scaled with darker color representing higher score. Percentage of cytotoxic or exhausted cells in each population is represented by the circle size.
d. Quantification of fractional presentation of clusters of CD8+ T cells across two subtypes of tumors (n=2 per subtype).
e. UMAP of clusters of CD4+ T cells obtained from four AML tumors (downsampled to have equal number of cells from SLS or IS dominant tumors).
f. Violin plot of representative marker genes of each cluster of CD4+ T cells. The y-axis represents the normalized gene expression value.
g. Module score of T cell exhaustion or cytotoxicity across major CD4+ T cell population in SLS or IS dominant tumors calculated as C.
h. Quantification of fractional presentation of clusters of CD4+ T cells across the two subtypes of tumors (n=2 per subtype).
i. Representative shared T cell clonotypes identified in IS-dominant tumor and in SLS-dominant tumor. Each clonotype is represented by a different color. Major cell groups are display on left panel.
j. Shared TCR clonotypes in CD8+ T cells and CD4+ T cells, after normalizing to total cell numbers. Number of shared clonotypes between each pair of subtypes were displayed. *p<0.05, two-sided Fisher's exact test. No shared clonotypes were identified in CD4+ T cells in SLS-dominant tumor.
k. RNA velocity of T cell population calculated based on ratio of unspliced and spliced transcripts in each cell. (Left panel) velocity vectors represented by arrows indicate potential differentiation paths; (right panel) Quantitative analysis of RNA velocity of subtypes of T cells derived from IS versus SLS tumors.

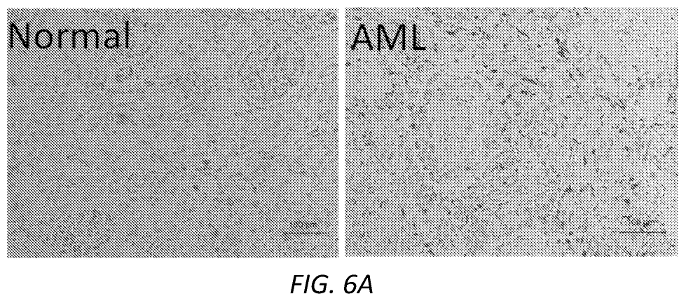

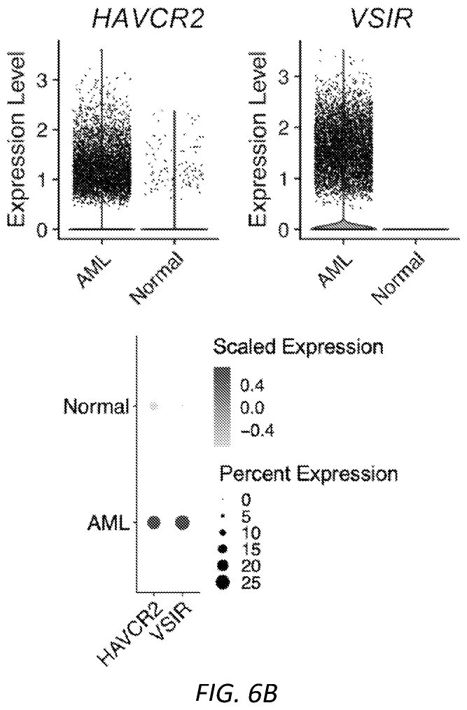

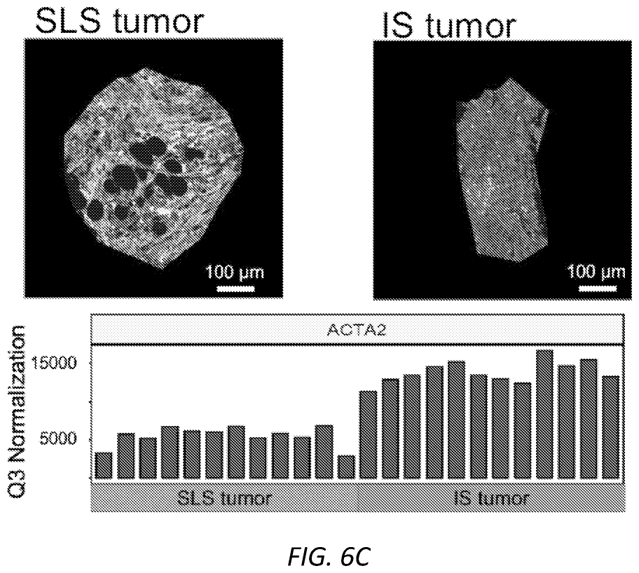

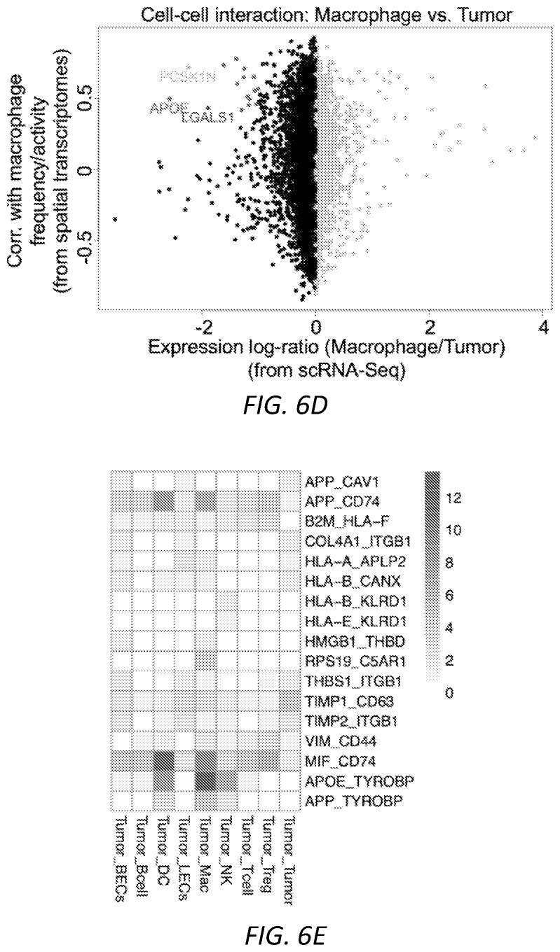

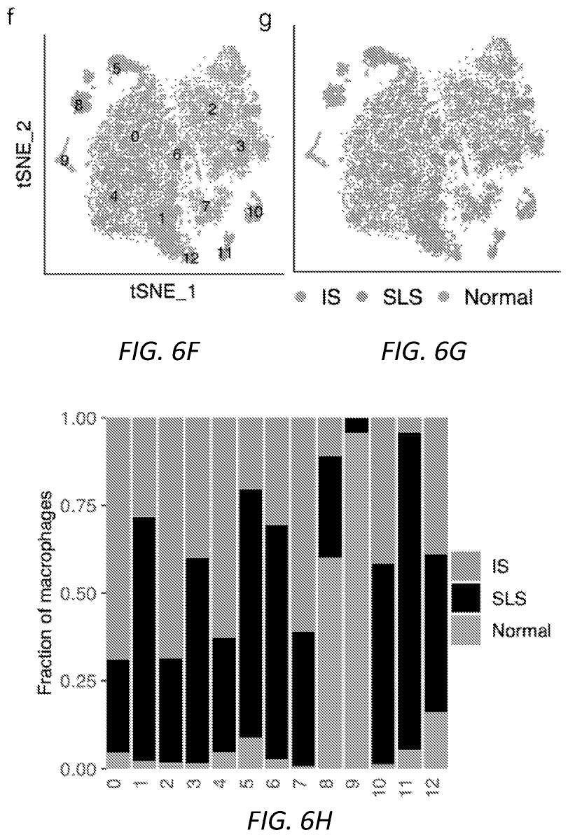

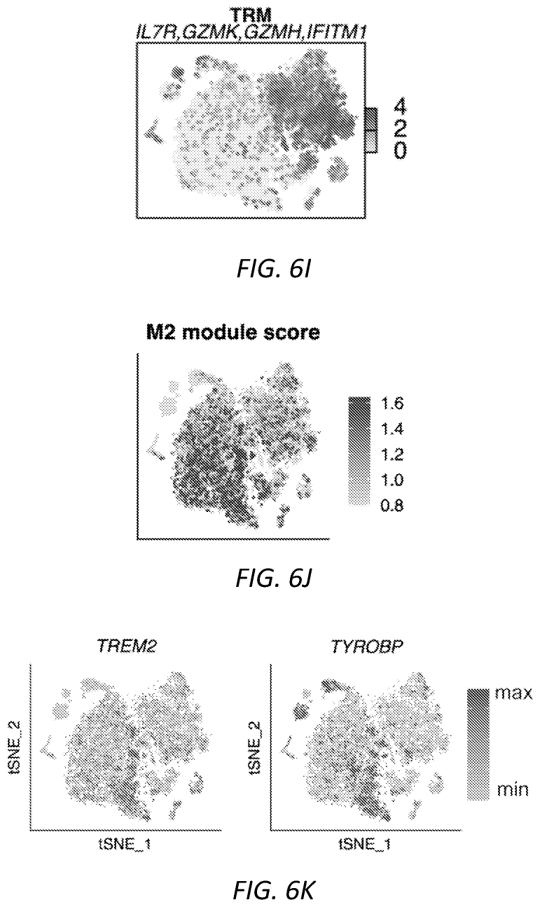

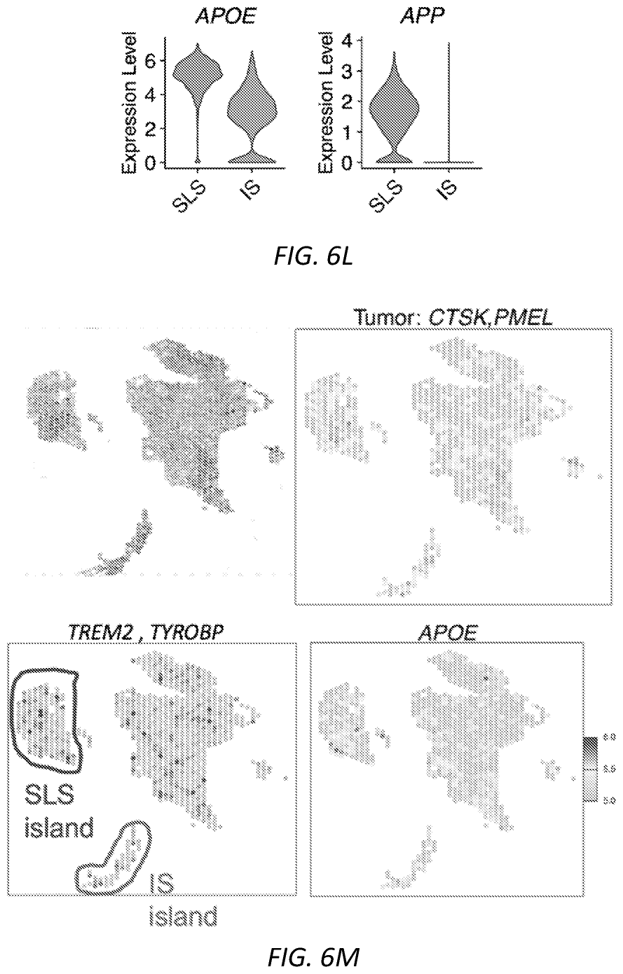

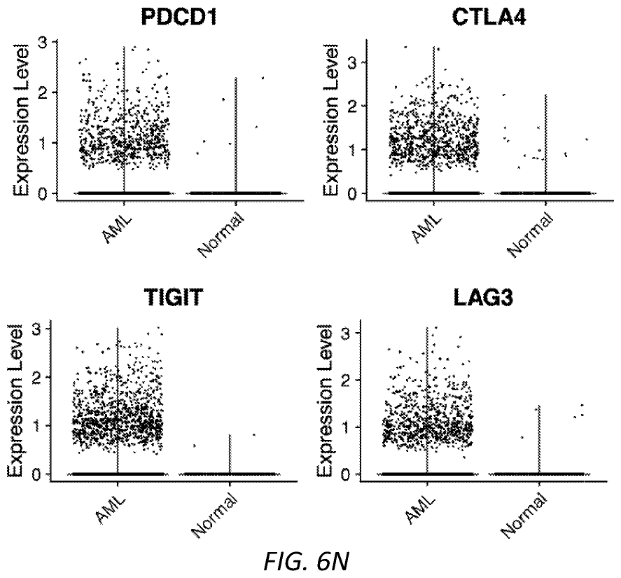

FIGS. 6A-N. The suppressive immune environment is shaped by tumor cell states.
a. Representative CD68 IHC staining of 5 AML tumors and 4 matched normal samples.
b. Higher expression of TIM3 (HAVCR2) and VISTA (VSIR) in macrophages obtained from tumors compared to macrophages obtained from matched normal kidneys. Left panel: violin plot showing expression of HAVCR2 and VSIR; right panel: dot plot showing scaled expression and the percentage of cells expressing these genes.
c. Nanostring digital spatial profiling of one SLS-dominant and one IS-dominant tumor. Left panel: a representative ROI (Region of Interests) from SLS-dominant tumor; middle panel: a representative ROI from IS-dominant tumor; right panel: expression of ACTA2 across all ROIs after Q3 normalization (From left to right columns are 12 SLS-dominant tumor ROIs and 12 IS-dominant tumor ROIs). Scale bars, 100 μm.
d. Inferred interactions between tumor cells and macrophages calculated by integrative analysis of spatial transcriptomics of the representative SLS-dominant tumor (12 ROIs) and scRNA-seq. x-axis displays relative expression of genes in single cell data. Only genes that are expressed in both single cell data and spatial transcriptomics data are shown. Left side are genes relatively highly expressed in tumor cells; right side are genes relatively highly expressed in macrophages. Y-axis displays Pearson Correlation Coefficient (PCC) of gene expression with macrophage frequency in spatial transcriptomics data. Genes with log-ratio less than-1.5 and correlation coefficient higher than 0.4 are colored. APOE: PCC=0.49, p=0.1 (correlation test, two-sided).
e. Interactions between tumor cells and other cell types calculated as the product of the average ligand expression and average receptor expression (only interactions with a score greater than 1 across any cell type pair are displayed). Each column shows a pair of cell types, and each row shows the ligand-receptor pair. The color indicates interaction score. Column label: cell type expressing the ligand and cell type expressing the receptor are separated by “_”. Row label: ligand and receptor are separated by “_”.
f. tSNE plot of macrophages colored by cluster (downsampled to have equal number of cells from SLS or IS tumors).
g. tSNE plot of macrophages colored by sample type of origin.
h. Fraction of macrophages obtained from subtypes of tumors or from matched normal across clusters.
i. Average expression of IL7R, GZMK, GZMH, and IFITM1.
j. M2 module score calculated by relative expression of CD163, MRCI, VEGFA, GPNMB and TREM2.
k. Feature plot showing expression of TREM2 and TYROBP (DAP12).
l. Violin plot showing expression of APOE and APP in tumor cells (SLS vs. IS).
m. Spatial transcriptomic profiling of an independent AML tumor using 10× Visium platform. Panels from left to right: 1) H&E stained tissue, 2) averaged expression of CTSK and PMEL; spots with expression of CTSK and PMEL higher than the median of all spots were annotated as tumor, 3) SLS spots and IS spots were identified by marker gene expression; averaged expression of TREM2 and TYROBP are displayed in red, 4) expression of APOE.
n. Violin plot of expression of immune checkpoint genes in macrophages obtained from tumors or from matched normal kidneys.

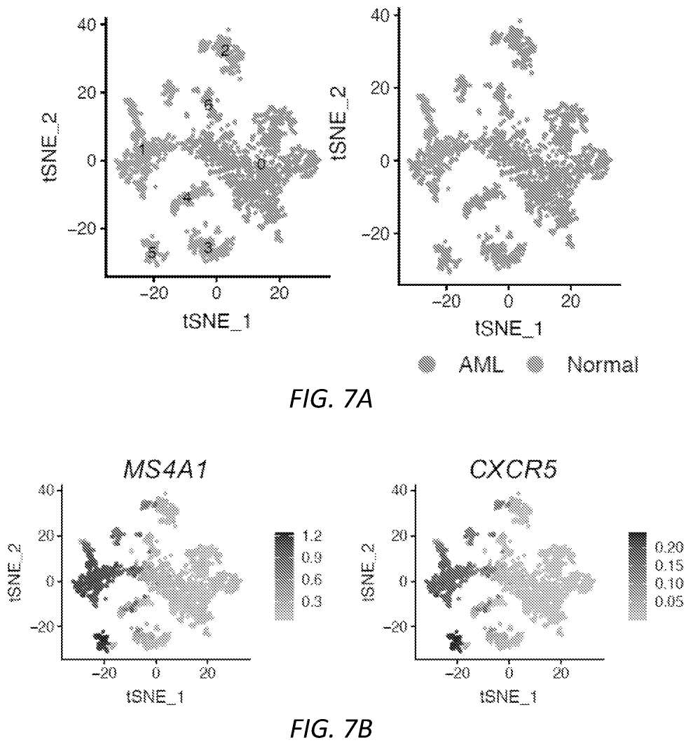

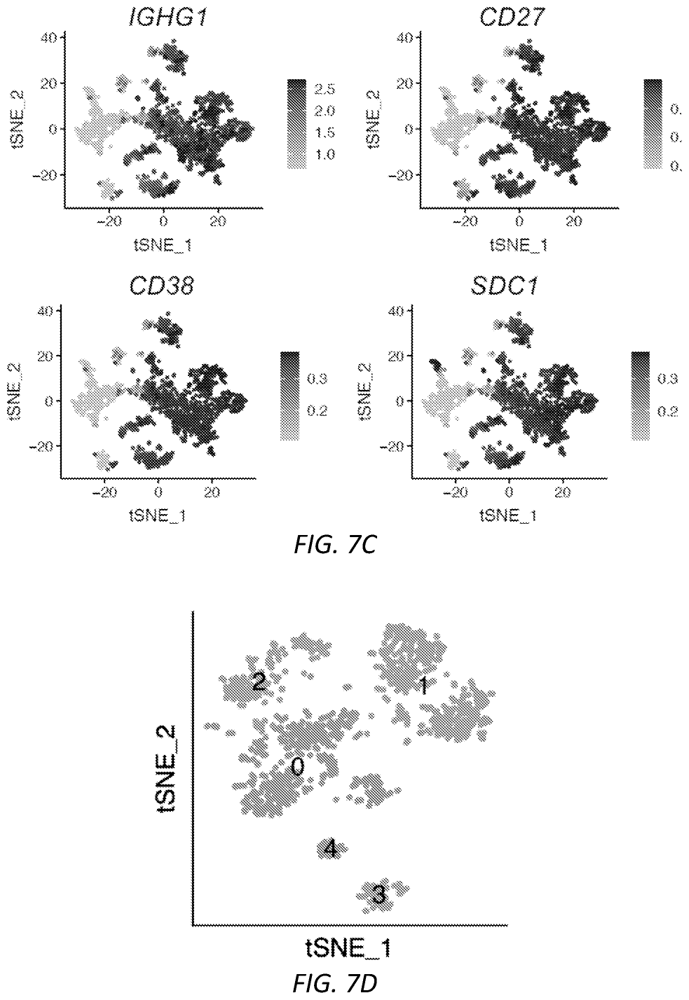

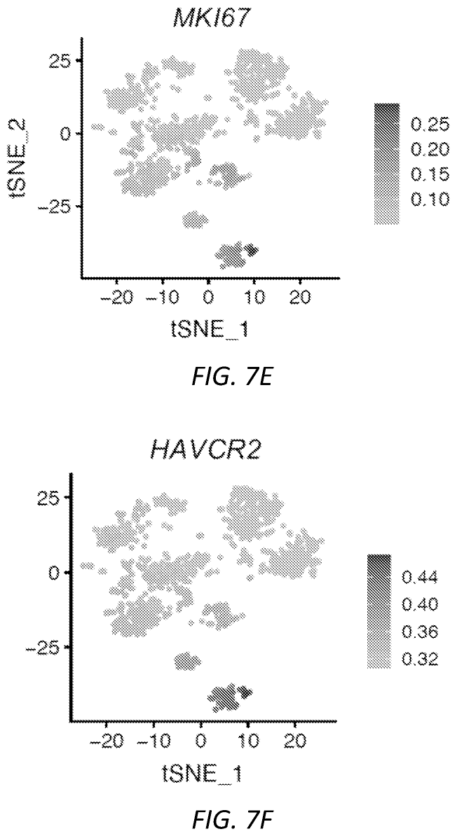

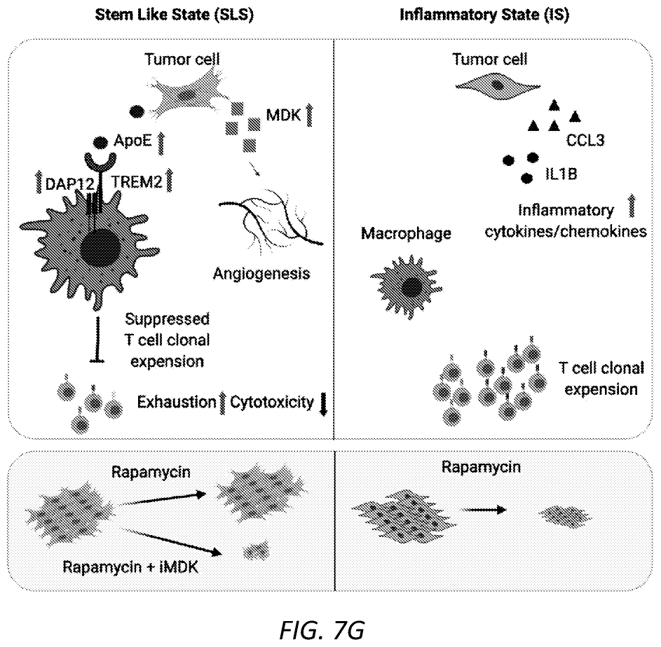

FIGS. 7A-G. Molecular interactions between tumor and immune compartment inferred by ligand-receptor co-expression.
a. tSNE plot of 1,620 B cells colored by cluster (left) or the origin (right).
b. Feature plot showing expression of follicular B cell marker genes MS4A1 and CXCR5.
c. Feature plot showing expression of plasma B cell marker genes.
d. tSNE plot of dendritic cells from AML tumors which are colored by cluster.
e. High expression of MKI67 in proliferating dendritic cells.
f. High expression of TIM3 (HAVCR2) in proliferating dendritic cells.
g. Schematic showing the main discoveries from this study: identification of two cell states (SLS and IS), their differential cellular ecosystem with active crosstalk between tumor cells and microenvironment, and association with rapamycin resistance and immune modulation. In SLS tumor cells, upregulated APOE may modulate tumor-associated macrophages toward an immune suppressive state by directly binding to TREM2/TYROBP receptor complex, leading to T cell dysfunction and diminished T cell clonal expansion; upregulated MDK expression may induce angiogenesis and drive persistence in response to mTORC1 inhibition. MDK is identified as a potential therapeutic target combining with rapamycin for persisting SLS tumor. In contrast, IS tumors with upregulated inflammatory pathways exhibit higher T cell cytotoxicity/proliferation and sensitivity to rapamycin treatment.

### DETAILED DESCRIPTION

The mTORC1 inhibitors sirolimus (rapamycin) and everolimus (Afinitor) are closely related compounds termed rapalogs, and are FDA-approved for the therapy of LAM and AML, respectively. Rapalogs induce a modest response in most patients with a median 50% volume reduction of AML⁶ and stabilization of lung function in LAM for at least 12 months⁷, with recurrent tumor growth and lung function decline after treatment cessation. Therapeutic strategies that eliminate, rather than suppress, tumor cells in TSC, are urgently needed.

Prior efforts to characterize TSC tumors using bulk RNA-Sequencing (RNA-Seq) has advanced our understanding of the unique transcriptional programs of TSC tumors⁸, including the important role of Melanocyte Inducing Transcription Factor (MITF)⁹, but were limited in the ability to reveal tumor cell heterogeneity, or interaction between tumor and microenvironment⁸. In contrast, single cell RNA-Sequencing (scRNA-Seq) enables comprehensive investigation of heterogeneity of tumor and microenvironment cells and global mapping of molecular interactions among cell types. Two recent single cell studies on LAM lungs have yielded important insight into the cellular origin of LAM cells and revealed alveolar epithelial remodeling by LAM cells^{10,11}. However, these studies were limited by the small number of LAM cells identified (<200 LAM cells).

Tumor cell heterogeneity and plasticity is increasingly recognized as an important and common aspect of tumor biology. The occurrence of multiple cell states in tumors and plasticity of inter-conversion of cell states likely contributes to therapeutic resistance¹². In AML, three different cell types represent the neoplastic process (fat, muscle, and vessels)¹³. Cellular heterogeneity is evident in both AML and LAM, but the precise components of this heterogeneity, how the different cellular elements inter-relate, and how each element responds to therapy are unexplored. In addition, aberrant vascular hypertrophy is also typical of AML¹³, and may contribute to an hypoxic tumor microenvironment. Tumor cells can acquire stemness and dormancy due to hypoxic conditions, and become stress and therapy resistant¹⁴.

Emerging data suggest that the immune system plays a key role in the pathogenesis and potentially the therapy of LAM and AML. Natural killer cells are enriched and activated in LAM^{15,16} Evidence of T cell infiltration and exhaustion have been observed in human AML and LAM and in mouse models, and there is clear benefit of immunotherapy in mouse models of TSC and LAM^{17,18}. This T cell infiltration and dysfunction are unexpected since AML have a very low neoantigen burden¹⁹. Macrophage infiltration was also observed in renal AML²⁰, hepatic AML²¹ and TSC skin tumors²². Despite these advances in understanding the immune microenvironment of LAM and AML, a comprehensive analysis has not been possible. In addition, the identification of molecular interactions between AML/LAM tumor cells and other cell types in the microenvironment has not previously been possible.

To address these points, we interrogated the tumor microenvironment of AML and LAM. Single cell profiling of 5 LAM specimens, 6 AML and 4 matched normal kidneys revealed two distinct cell states in AML/LAM cells: a stem-like state (SLS) and an inflammatory state (IS). SLS tumor cells exhibited high stemness and dormancy marker expression, and showed rapamycin resistance in primary angiomyolipoma-derived cultures. Midkine (MDK) was highly expressed specifically in SLS cells, and MDK inhibitor treatment enhanced the therapeutic effect of rapamycin in patient-derived TSC2-deficient AML cells in vitro and in vivo. Integrative analysis of single cell data and spatial transcriptomic profiling of these tumors further revealed a modulatory axis from SLS tumor cells to suppressive TREM2+/TYROBP+ macrophages, leading to T cell dysfunction. Concurrent single cell T cell receptor sequencing (scTCR-Seq) analysis demonstrated a substantial suppression of clonal expansion and T cell RNA velocity in SLS-dominant tumors compared to IS-dominant tumors. In contrast, inflammatory state (IS) tumor cells with low MDK expression showed high expression of cytokines and were enriched with immune regulatory pathways. Substantial T cell clonal expansion with elevated cytotoxic programs was observed in IS-dominant tumors compared with SLS-dominant tumors. Taken together, these data reveal differential immune remodeling by previously unrecognized distinct cells states in mTORC1-hyperactive tumors, and provide a rationale for precision immunotherapy in TSC.

mTORC1, a protein complex made up of comprised of mTOR, raptor, GBL and deptor,¹¹⁸ is estimated to be hyperactive in at least half of all human malignancies and plays a central role in tumorigenesis¹⁰⁵⁻¹⁰⁷. The present work provides a comprehensive atlas of tumor cells and the tumor microenvironment in mTORC1 hyperactive AML and LAM. Our analysis highlights a complex cellular ecosystem with active crosstalk between AML cells and the tumor microenvironment and distinct AML/LAM cell states associated with rapamycin resistance and immune modulation (FIG. 7h). In addition to confirming known genes and pathways contributing to TSC pathogenesis, this work highlights previously unrecognized pathways that likely contribute to tumor progression, and pinpoint targets for the future of immunotherapy in TSC. This study represents an important step toward understanding intra-tumoral expression heterogeneity in mesenchymal tumors, a far less studied tumor type than epithelial tumors.

Among the key findings is the identification of a conserved drug resistant tumor cell state characterized by stemness and dormancy seen in both AML and LAM. Rapamycin and its analogs induce a cytostatic effect in TSC treatment, resulting in some shrinkage and then stabilized tumor volume. Here, we reveal two distinct cell states (SLS and IS) in the tumor cell population, and identify underlying transcription factors that appear to drive the development of these different cell states in response to the tumor microenvironment, characterized by distinct expression of tumor stem cell and dormancy programs or inflammatory programs. Immunofluorescent staining confirmed the existence of these cell states, as predicted by single cell transcriptomic profiling. SLS cells with stemness and dormancy properties contribute to rapamycin tolerance as shown by our in vitro treatment analyses. Inhibition of MDK, a gene highly expressed in SLS cells, enhanced rapamycin's therapeutic effect both in vitro and in vivo, suggesting that MDK may at least partially account for the molecular mechanism of rapamycin tolerance in TSC, in line with role of MDK in drug resistance observed in other cancers^{77,108}. Thus, intra-tumoral heterogeneity, which is believed to underlie therapy resistance in many malignant tumors, also occurs in mTORC1-hyperactive AML and LAM, and combinatorial targeting of mTORC1 and factors such as MDK that contribute to this heterogeneity may enhance the efficacy of mTORC1 inhibition.

SLS-dominant tumors were enriched for both blood endothelial cells and lymphatic endothelial cells when compared to IS-dominant tumors, indicating differential induction of vascular remodeling of endothelial cells. We validated this enrichment of endothelial cells by IHC. Lymphatic vascularization is a hallmark of both AML and LAM, AML cells can metastasize to regional lymph nodes, and it has been proposed that LAM cells metastasize to the lungs from a distant unknown site-of-origin^{24,109} VEGFD is thought to promote lymphangiogenesis and lymphatic metastasis²⁴. Serum VEGFD levels are elevated in about two-thirds of LAM patients, serving as an important diagnostic biomarker¹¹⁰. Whether other growth factors may contribute to lymphangiogenesis in LAM, including the one-third of LAM patients without elevated VEGF-D, is a critical unanswered question. We identified MDK as a secreted factor that may promote lymphangiogenesis and angiogenesis in SLS-dominant tumors, and found that MDK is elevated in the serum of LAM patients, suggesting that it may be a critical mechanistic link to lymphangiogenesis in LAM as well as a candidate therapeutic target.

Compared to matched normal kidneys, a higher percentage of T cells was observed in AML tumors, and proliferating T cells were solely observed in tumors, indicating tumor-induced T cell activation and expansion. This concept is supported by increased expression of genes associated with inflammation in tumor-associated T cells revealed by comparative pathway analysis. This T cell infiltration in tumors was validated by IHC and supports the conclusion of a prior study of T cells in AML¹⁷. Evidence of T cell exhaustion was present in the effector T cell population, consistent with T cell exhaustion previously reported in human AML and LAM and in mouse models^{17,18}, which may curtail the proliferation and cytotoxicity of tumor-recognizing T cells¹¹¹. Intriguingly, CD8+ T cells derived from SLS-dominant exhibited much higher exhaustion and lower cytotoxicity compared to those from IS-dominant tumors. Integrative analysis of paired scRNA-Seq and scTCR-Seq revealed that clonal expansion and T cell velocity were almost completely suppressed in SLS-dominant tumors.

We observed striking macrophage infiltration in these renal AML, validated by IHC and consistent with previous observations in hepatic AML²¹, emphasizing a possible role of the innate immune system in TSC. M2 polarization of TAMs is implicated in tumor promotion and immune suppression¹¹². A subset of M2-like TAMs was observed in AML, characterized by high expression of M2 marker genes. Interestingly, it seems that macrophage alternative polarization in AML tumors is shaped by different tumor cell states. Specifically, SLS-dominant tumors were enriched with M2-like macrophages with high expression of TREM2 and TYROBP, a receptor complex on macrophages recently shown to suppress T cell function in tumor microenvironment^{96,97}. Because TREM2+/TYROBP+ tumor-infiltrating macrophages inhibit T cell proliferation in animal models of sarcoma, colorectal cancer, and mammary tumor^{96,97}, it is possible that these suppressive macrophages are responsible for the observed T cell dysfunction and almost complete suppression of T cell clonal expansion and differentiation observed in SLS-dominant tumors. Integrative analysis of spatial transcriptomic profiling and single cell analysis identified a connection between APOE (primarily expressed by tumor cells) and macrophage population frequency, which was robustly recapitulated by a further integrative analysis of bulk RNA-Seq and single cell analysis. Genome-wide ligand-receptor analysis revealed APOE-TYROBP as the strongest tumor-microenvironment interaction, suggesting a regulatory axis from tumor cells to suppressive TAMs. The TREM2/TYROBP complex acts as a receptor for amyloid-beta protein 42, a cleavage product of the amyloid-beta precursor protein APP⁹³ and APOE¹¹³. Consistently, both APP and APOE showed higher expression in SLS AML cells compared to IS type. Since expression of known immune checkpoint ligands was extremely low on tumor cells in both SLS- and IS-dominant tumors, T cell function and proliferation/differentiation may be inhibited indirectly by the SLS tumor cells via induced suppressive TAMs. While tumor mutation burden has been associated with response to immune checkpoint therapy in multiple cancer types, it is not a perfect marker of response, and suppressive myeloid cells have gained attention as a critical determinant of therapeutic resistance in multiple cancer types¹¹⁴. This work suggests that in TSC tumors, which are known to have an extremely low mutational burden¹⁹, suppressive myeloid cells may drive immune suppression, and blocking tumor-myeloid cell crosstalk may enhance immune regulation of these tumors.

Thus, provided herein are methods for treating Tuberous Sclerosis Complex (TSC)-associated diseases, e.g., angiomyolipoma (AML) and lymphangioleiomyomatosis (LAM), by administering a combination of therapeutic agents targeting mTORC1 and therapeutic agents targeting MDK, to enhance the efficacy of mTORC1 inhibition.

### Methods of Treatment

The methods described herein include methods for the treatment of Tuberous Sclerosis Complex (TSC)-associated benign and malignant tumors. In some embodiments, the TSC-associated tumor is a cortical dysplasia, subependymal nodule, subependymal giant cell astrocytoma (SEGA), cardiac rhabdomyoma, dermatologic or ophthalmic tumor, renal cyst, multifocal micronodular pneumocyte hyperplasia (MMPH), splenic hamartoma, perivascular epithelioid tumors (PEComas), lymphangioleiomyomatosis (LAM), or angiomyolipoma (AML); Also provided herein are methods for the treatment of LAM and AML in the absence of a diagnosis of TSC or of mutations in the TSC1 or TSC2 tumor suppressor genes, e.g., LAM/AML that occur sporadically. Methods for identifying subjects are known in the art (see, e.g., Wang et al., RadioGraphics 2021 41:7, 1992-2010). Generally, the methods include administering a therapeutically effective amount of a treatment as described herein, to a subject who is in need of, or who has been determined to be in need of, such treatment. In some embodiments, the methods include administering a therapeutically effective amount of a treatment comprising an agent that inhibits mTORC1 and an agent that inhibits MDK. The methods can also optionally include administering an immunotherapy (e.g., a checkpoint inhibitor), or a standard treatment comprising chemotherapy, radiotherapy, and/or resection.

As used in this context, to “treat” means to ameliorate at least one symptom of the disorder. For example, a treatment can result in a reduction in tumor size or growth rate, a reduction in risk or frequency of reoccurrence, a delay in reoccurrence, a reduction in metastasis, increased survival, and/or decreased morbidity and mortality, inter alia.

mTORC1 Inhibitors

A number of mTORC1 inhibitors are known in the art and include, but are not limited to, ATP-competitive mTORC1 inhibitors, e.g., MLN0128, MHY1485, PI-103, PP242 (torkinib), PP30, XL388, AZD2014 (vistusertib), voxtalisib (SAR24540; XL765), vistusertib, OSI-227, WAY-600, WYE-132, WYE-687, or sapanisertib (TAK-228); PF-04691502; Gedatolisib (PKI-587; PF05212384); AZD 8055 ((5-(2,4-bis((S)-3-methylmorpholino)pyrido[2,3-d]pyrimidin-7-yl)-2-methoxyphenyl) methanol); Torin-1 (1-[4-[4-(1-oxopropyl)-1-piperazinyl]-3-(trifluoromethyl)phenyl]-9-(3-quinolinyl)-benzo[h]-1,6-naphthyridin-2 (1H)-one); torin-2; apitolisib; gedatolisib; GSK2126458 (GSK458); CC-223; FKBP12 enhancers; 4H-1-benzopyran-4-one derivatives; and rapamycin (also known as sirolimus) and derivatives thereof, including: temsirolimus (Torisel®), umirolimus, everolimus (Afinitor®; WO94/09010) ridaforolimus (also known as deforolimus or AP23573), and zotarolimus; rapalogs, e.g., as disclosed in WO98/02441 and WO01/14387, e.g. AP23464 and AP23841; 40-(2-hydroxyethyl) rapamycin; 40-[3-hydroxy (hydroxymethyl)methylpropanoate]-rapamycin (also known as CC1779); 40-epi-(tetrazolyt)-rapamycin (also called ABT578); 32-deoxorapamycin; 16-pentynyloxy-32 (S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). Other mTORC1 inhibitors include cornarin A, dactolisib, omipalisib, samotolisib, KU-0063794, gadatolisib, dactosulib tosylate, CC-115, apitolisib, bimarilisib, VS-5584, GDC-0349, CZ415, WYE-354, onatasertib, mTOR-inhibitor 3, palomid 529, PQR620, (+)-usnic acid, MT 63-78, MTI-31, FT-1518, and AZD3147. In some embodiments, the mTOR inhibitor is a bisteric inhibitor (see, e.g., WO2018204416, WO2019212990 and WO2019212991), such as RMC-5552. See also Hua et al., Targeting mTOR for cancer therapy. J Hematol Oncol 12, 71 (2019); Wolin et al., A phase 2 study of an oral mTORC1/mTORC2 kinase inhibitor (CC-223) for non-pancreatic neuroendocrine tumors with or without carcinoid symptoms. PLoS One. 2019 Sep. 17; 14 (9): e0221994; Moore et al., Phase I study of the investigational oral mTORC1/2 inhibitor sapanisertib (TAK-228): tolerability and food effects of a milled formulation in patients with advanced solid tumours. ESMO Open. 2018 Feb. 1; 3 (2): e000291. Many of the above are commercially available, e.g., from MedChemExpress.

### MDK Inhibitors

A number of MDK inhibitors are known in the art and include, but are not limited to, iMDK (3-(2-(4-Fluorobenzyl) imidazo[2,1-b]thiazol-6-yl)-2H-chromen-2-one, 3-(2-(4-Fluorobenzyl) imidazo[2,1-b][1,3]thiazol-6-yl)-2H-chromen-2-one, available, e.g., from Axon Medchem or Calbiochem)), see, e.g., Hao et al., PLoS One. 2013 Aug. 16; 8 (8): e71093, as well as anti-midkine antibodies (see, e.g., U.S. Pat. Nos. 10,590,192, 9,163,081, 9,624,294, EP 0998941, and US20140170144; RNA Aptamers (see for example Kishida and Kadomatsu, British Journal of Pharmacology (2014) 896-904, EP2924120, WO2008059877), and inhibitory nucleic acids targeting midkine, e.g., antisense oligonucleotides (e.g., morpholino oligonucleotides), siRNA, or shRNA (see, e.g., US20110159022, WO2018016674, Kishida and Kadomatsu, British Journal of Pharmacology (2014) 896-904). Exemplary sequences of human midkine are as follows:

| Nucleic acid | Protein | Name/variant |
|---|---|---|
| NM_001012334.2 | NP_001012334.1 | midkine isoform a precursor - variant 1 |
| NM_001012333.3 | NP_001012333.1 | midkine isoform a precursor - variant 2 |
| NM_001012333.3 | NP_001012333.1 | midkine isoform a precursor - variant 3 |
| NM_001270550.1 | NP_001257479.1 | midkine isoform a precursor - variant 4 |
| NM_001270551.3 | NP_001257480.1 | midkine isoform a precursor - variant 5 |
| NM_001270552.3 | NP_001257481.1 | midkine isoform b precursor - variant 6 |

Additional inhibitors can be identified, e.g., using methods described in Matsui T, Ichihara-Tanaka K, Lan C, Muramatsu H, Kondou T, Hirose C, Sakuma S, Muramatsu T. Midkine inhibitors: application of a simple assay procedure to screening of inhibitory compounds. Int Arch Med. 2010 Jun. 21; 3:12. See also Muramatsu T. Midkine: a promising molecule for drug development to treat diseases of the central nervous system. Curr Pharm Des. 2011; 17 (5): 410-23.

### Checkpoint Inhibitors

The present methods can include administering an immunotherapy comprising a checkpoint inhibitor, e.g., an inhibitor of PD-1 signaling, e.g., an antibody that binds to PD-1, CD40, or PD-L1, or an inhibitor of Tim3 or Lag3, e.g., an antibody that binds to Tim3 or Lag3, or an antibody that binds to CTLA-4, or an antibody that binds to T-cell immunoglobulin and ITIM domains (TIGIT).

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; and U.S. Pat. No. 8,591,900, including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3:15-25 (2010); Storz, MAbs. 2016 January; 8 (1): 10-26; US2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab, Tremelimumab, and EPR1476.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP_116171.3. Exemplary antibodies are described in WO2016071448; U.S. Pat. No. 8,552,156; and US PGPub. Nos. 20180298097; 20180251549; 20180230431; 20180072804; 20180016336; 20170313783; 20170114135; 20160257758; 20160257749; 20150086574; and 20130022623, and include LY3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 March; 276 (1): 80-96; Antoni et al., Am Soc Clin Oncol Educ Book. 2016; 35: e450-8; US PGPub. Nos. 20180326054; 20180251767; 20180230431; 20170334995; 20170290914; 20170101472; 20170022273; 20160303124, and include BMS-986016.

Exemplary anti-TIGIT antibodies that can be used in the methods described herein include those that bind to human TIGIT; an exemplary human TIGIT sequence is provided at NCBI Accession No. NP_776160.2. Exemplary antibodies include AB154; MK-7684; BMS-986207; ASP8374; Tiragolumab (MTIG7192A; RG6058); (Etigilimab (OMP-313M32)); 313R12. See, e.g., Harjunpää and Guillerey, Clin Exp Immunol 2019 Dec. 11 [Online ahead of print], DOI: 10.1111/cei.13407; 20200062859; and 20200040082.

### Pharmaceutical Compositions and Methods of Administration

The methods described herein include the use of pharmaceutical compositions comprising or consisting of an inhibitor of mTORC1 and an inhibitor of MDK as an active ingredient. In some embodiments, the inhibitor of mTORC1 and inhibitor of MDK are in a single composition; in some embodiments, the inhibitor of mTORC1 and inhibitor of MDK are in separate compositions. In some embodiments, no other active compounds are present in the composition(s); in some embodiments, no other active compounds are administered

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88 (2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a kit, container, pack, or dispenser, optionally with instructions for administration, for use in a method described herein.

### Methods of Identifying Subjects for Immunotherapy Treatment

Included herein are methods for identifying and selecting subjects for treatment with an immunotherapy, e.g., with a checkpoint inhibitor. The methods rely on detection of MDK (e.g., MDK protein or mRNA) in a sample from the subject. The methods include obtaining a sample from a subject, and evaluating the presence and/or level of MDK in the sample, and comparing the presence and/or level with one or more references, e.g., a control reference that represents a threshold level of MDK associated with the presence or absence of an immunosuppressive tumor environment.

As used herein the term “sample”, when referring to the material to be tested for the presence of a biological marker using the method of the invention, includes inter alia tissue (e.g., a tumor sample), whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid. The type of sample used may vary depending upon the clinical situation in which the method is used. In some embodiments, the sample is serum and MDK protein is measured. Various methods are well known within the art for the identification and/or isolation and/or purification of a biological marker such as MDK from a sample. An “isolated” or “purified” biological marker is substantially free of cellular material or other contaminants from the cell or tissue source from which the biological marker is derived, i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84 (14): 6008-6015; Brody (2010) Expert Rev Mol Diagn 10 (8): 1013-1022; Philips (2014) PLoS One 9 (3): e90226; Pfaffe (2011) Clin Chem 57 (5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. Nos. 5,118,937; 5,045,694; 5,719,060; 6,225,047)

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48 (8): 1178-1185; Miranda (2010) Kidney International 78:191-199; Bianchi (2011) EMBO Mol Med 3:495-503; Taylor (2013) Front. Genet. 4:142; Yang (2014) PLoS One 9 (11): e110641); Nordstrom (2000) Biotechnol. Appl. Biochem. 31 (2): 107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289 (5485): 1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of MDK. Measurement of the level of a biomarker can be direct or indirect. For example, the abundance levels of MDK can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such as beta actin for example, can be used.

Gene arrays can be prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, the level of MDK is comparable to or above the threshold level of the protein(s) (e.g., in the control reference or in a healthy tissue of the same kind of tissue from the same subject), then the subject can be identified as unlikely to have an immunosuppressive tumor environment, and as likely to respond to treatment with an immunotherapy. In some embodiments, once it has been determined that a person is unlikely to have an immunosuppressive tumor environment, then a treatment comprising an immunotherapy and an mTORC1 inhibitor, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of MDK, e.g., a control reference level that represents a level of MDK associated with presence or absence of an immunosuppressive tumor environment (e.g., wherein levels above the reference indicate the absence of an immunosuppressive tumor environment, and levels below indicates the presence of an immunosuppressive tumor environment.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with presence or absence of an immunosuppressive tumor environment in one defined group is a fold higher, or lower, (e.g., approximately 2-fold, 4-fold, 8-fold, 16-fold or more) than in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point, or in a tissue of the same type (from the same organ) that is normal (not tumor tissue).

In characterizing likelihood, or risk, numerous predetermined values can be established.

### EXAMPLES

The invention is further described in the following examples, which do not limit the scope of the invention described in the claims.

### Materials and Methods

The following materials and methods were used in the examples below.

### EXPERIMENTAL MODEL AND SUBJECT DETAILS

### Patient Samples

LAM specimens, AML tumor samples and matched normal kidneys were collected under IRB approved by the Brigham and Women's Hospital. All patients provided informed written consent. None of these patients received rapalog treatment for six months prior to surgery. AML samples were obtained locally from Massachusetts General Hospital, Brigham and Women's Hospital/Dana-Farber Cancer Institute or Beth Israel Deaconess Medical Center in Boston. LAM samples were obtained either locally from Brigham and Women's Hospital or from National Disease Research Interchange (NDRI). All samples were immediately dissociated and subjected to single cell analysis upon receipt. No specific sampling was performed for AML or LAM samples. The entire piece was analyzed for scRNA-seq.

### Cell Lines

The following cell lines were maintained in our lab: patient-derived TSC2-deficient cell line 621-101, TSC2-addback cell line 621-103, mouse kidney derived TSC2-null cell line TTJ (the gift of Vera Krymskaya) and TSC2-add back cell line TTJ-TSC2. The normal human lung fibroblasts NHLF (CC-2512) was purchased from Lonza Group (Switzerland). All cells were cultured in DMEM supplemented with 10% FBS (Thermofisher Scientific), and were cultured at 37° C. in a humidified chamber with 5% CO₂ during the experiments. TSC2-null and TSC2-add back cell lines are routinely authenticated using western immunoblotting and qPCR to confirm TSC2 protein loss and expression before any experiment. All cells used in this study were tested negative for mycoplasma contamination. All cells in our laboratory are monthly tested for mycoplasma contamination.

### Mice

Animal studies were approved by the Brigham and Women's Hospital Animal Care and Use Committee (IACUC). All husbandry and experiment procedures with mice were conducted in accordance with protocols approved. Mice were provided water and food ad libitum and were housed on a standard light (12h) and dark (12h) cycle at 72° F. and 40% humidity. Athymic nude mice (Crl: NU (NCr)-Foxn1^{nu}, Charles River Laboratories, Wilmington, MA) were all seven-week-old female mice at the time of TTJ cell injection for allograft experiments. In all mice experiments, maximal tumour sizes were not exceeded 1500 mm3, the maximal tumour size permitted by IACUC.

### Single Cell RNA-Seq

### Tissue Dissociation

Fresh tumor and matched normal samples were dissociated into single cell suspension using human tumor dissociation kit (Miltenyi Biotec) and gentleMACS™ Dissociator (Miltenyi Biotec), according to manufacture's manual. Red blood cells were removed by Red Blood Cell Lysis Solution kit (Miltenyi Biotec). Cell suspensions were washed with cold PBS. Viability of all samples were confirmed with trypan blue staining (Invitrogen) to be above 70% before loading to 10× Chromium controller.

### Single Cell RNA Sequencing

Droplet emulsions were immediately recovered for reverse transcription reaction using Bio-rad thermocycler. Single cell expression libraries were constructed using 10× genomics Chromium 5′ barcoding reagents (v1) following manufacturer's manual. Quality of amplified cDNA and constructed libraries were confirmed by BioAnalyzer (Agilent, High Sensitivity DNA Kit). Library sequencing was performed by NextSeq 500 (Illumina).

### Paired Single Cell TCR Sequencing

Aliquots of 2 μl amplified cDNA from the single cell expression library construction workflow were used for TCR library construction according to 10× genomics manufacturer's manual. Quality of amplified cDNA and constructed libraries were confirmed by BioAnalyzer (Agilent, High Sensitivity DNA Kit). Library sequencing was performed by NextSeq 500 (Illumina). Sequencing depth forV (D) J enriched libraries were at least 5000 read pairs per cell. Standard Illumina sequencing primers were used for both sequencing and index reads following 10× manufacturer's protocol.

### Nanostring Whole Transcriptome Digital Spatial Profiling

Two tumors were profiled using Nanostring digital spatial profiler for whole transcriptome analysis. Resected tumor samples were washed with cold PBS and fixed in 50 ml 10% Formalin for 24 hours before embedding. All antibody staining and whole transcriptome sequencing were performed on freshly cut FFPE slides. Regions of interest (ROIs) were selected based on immunofluorescence staining using Nanostring validated antibodies against α-SMA obtained from Invitrogen (clone 1A4, Cat. 53-9760-82, 1:400), CD3e obtained from Origene (clone UMAB54, Cat. UM500048, 1:200) and CD68 obtained from Santa Cruz (clone KP1, Cat. sc-20060AF594, 1:400). Each ROI was uniquely indexed then pooled for sequencing. Sequencing data were Q3 normalized by a standard Nanostring pipeline. Q3 (3rd quartile of all selected targets) normalization was used for all targets that are above the limit of quantitation. Q3 normalization uses the top 25% of expressed genes to normalize across ROIs/segments.

### 10× Visium Spatial Transcriptomics Profiling

Fresh tumor sample and matched normal kidney were immediately OCT embedded within 1 hour after surgery. OCT blocks was cryosectioned at −10° C. and placed on chilled Visium tissue optimization slide (PN: 3000394, 10× Genomics) or spatial gene expression slide (PN: 2000233, 10× Genomics). Slides were kept chilled during sectioning and transportation processes. H&E staining, tissue optimization and gene expression library construction were performed as per manufacturer's manual. Briefly, tissue permeabilization time was set to 18 minutes for gene expression experiment after time-course optimization experiment following manufacturer's protocol. Brightfield H&E images were taken using Keyence BZX800 microscope with a 20× objective. Images were stitched by Keyence BZX800 stitching function. Fluorescent images were taken with dsRed2 filter cube from Chroma Technology (ex/em: 545/30, 620/60) using a 10× objective. Libraries were sequenced on illumina NovaSeq 6000 at 300 pM concentration.

### Multiplex Immunofluorescence, RNA In Situ Hybridization and Immunohistochemistry

FFPE tissue blocks were freshly cut at thickness of 5 μm. The following primary antibodies were used for multiplex immunofluorescence staining performed by iHisto company: MDK [EP1143Y] (ab52637, Abcam, 1:100) (FITC labeled), TAGLN (ab14106, Abcam, 1 ug/ml) (cy5 labeled, red) and CTSK (PB9856, Boster, 0.5 μg/ml) (cy3 labeled, pseudo-colored for visualization). Nuclei were stained with DAPI. Slide images were scanned at 10× magnification.

RNA in situ hybridization (ISH) was performed at Brigham and Women's Hospital Pathology Core according to ACD user manual using RNAscope® 2.5 LS Probe-Hs-MDK-01 (586478, ACD, Bio-techne). All RNA in situ hybridization experiments were performed on the same samples subjected to single cell analysis.

All immunohistochemistry staining were performed at Brigham and Women's Hospital Pathology Core with validated antibodies against CD68 (clone PG-M1, M0876, Dako, 1:100), CD3 (clone F7.2.38, A0452, Dako, 1:50), CD31 (clone JC70A, M0823, Dako, 1:50).

### MDK ELISA Assay

Human and mouse MDK ELISA assays were performed using Human Midkine ELISA Kit PicoKine™ (EK1235, BOSTER) and Mouse MDK/Midkine (Sandwich ELISA) ELISA Kit (LS-F12048-1, LSBio) respectively following manufacturers' manuals. Standards were prepared immediately prior to performing the experiment. For human patient samples, frozen serum samples were thawed to room temperature and centrifuged at 15 minutes at 1000×g immediate before assessment. For cell culture assays, 621-101 cells, 621-103 cells, TTJ cells and TTJ-TSC2 cells were cultured to 90% confluence. Culture supernatants were collected, centrifuged at 500×g for 5 minutes, and assayed immediately.

### Quantitative PCR Assay

Probes Hs00171064_ml (human, ThermoFisher), Mm00440280_g1 (mouse, ThermoFisher), and TaqMan™ Universal Master Mix II, with UNG kit (4440038, ThermoFisher) were used for Quantitative PCR assays.

### Allograft Tumors

Seven-week-old female athymic nude mice (Crl: NU (NCr)-Foxn1^{nu}, Charles River) were subcutaneously injected with 3 million TTJ cells (75 μl cells mixed with 75 μl Matrigel, Corning 356237) on the front flank. All treatments started at day 8 after tumor inoculation when average tumor volume reached around 300 mm³. Mice were randomized in groups for treatment. Mice were treated 3 times per week for a total of 11 treatments with intraperitoneal injections of DMSO vehicle, the small molecule 3-[2-[(4-Fluorophenyl)methyl]imidazo[2,1-b]thiazol-6-yl]-2H-1-benzopyran-2-one (iMDK; TOCRIS Bio-techne, 9 mg/kg), rapamycin (Sirolimus A8167, APExBIO; 3 mg/kg), or combined iMDK (9 mg/kg) and rapamycin (3 mg/kg). Tumor volume was measured immediately before each treatment using a caliper.

Rapamycin Treatment and scRNA-Seq of Tumor-Derived Primary Culture

Resected tumor tissue was dissociated into single cell suspension as described above. Aliquots of 100 μl were dispensed into 10 cm dishes with fresh DMEM supplemented with 10% FBS. Primary cultures were maintained at 37° C. in a humidified chamber with 5% CO₂ for 2 weeks to allow to reach 80% confluence. Fresh media were changed every 3 days. Primary cultures were treated with rapamycin (20 nM) or vehicle for 24 hours before subjecting to droplet scRNA sequencing as described above.

Cell Line Estradiol Treatment and scRNA-Seq

Patient-derived TSC2-deficient 621-101 cells were grown in phenol-free DMEM supplemented with 10% charcoal-stripped FBS for 72 hours, then treated with 100 nM estradiol or ethanol vehicle for 24 hours, and subjected to single cell RNA sequencing as described above.

### Combination Treatment and Proliferation Assay of Cell Lines

621-101 cells, TTJ cells, or normal human fibroblasts NHLF cells were seeded in 12-well plates in DMEM with 10% FBS at 20-30% confluency. Cells were treated with DMSO (control), iMDK (1 μM), rapamycin (20 nM), or combined iMDK (1 μM) and rapamycin (20 nM) until the control group reached over 100% confluency. Drugs were refreshed every 2 days to ensure maximum activity. Cell proliferation was assessed using Crystal violet Assay Kit (Cell viability) (ab232855, abcam).

### Quantification and Statistical Analysis

Statistical analyses were performed with R, MATLAB, or GraphPad Prism (GraphPad Software). Statistical parameters are reported at appropriate places in main text, supplemental materials, figures and figure legends, including sample numbers, measures of center, standard deviation, or standard error (mean±SD or SEM), statistical significance.

### Single Cell RNA Sequencing Data Processing

Cell Ranger pipeline (10× Genomics) was used for reference genome alignment and generating gene-cell counts matrices. Raw sequencing data was aligned to GRCh38 reference genome using Cell Ranger pipeline (10× Genomics) to generate gene counts matrix by cell barcodes. Sequencing depth was on average 30,285 reads/cell. Data normalization and integration were performed using the Seurat (v4.0.2) R package³⁴. Cells were filtered from downstream analysis with the criteria of <200 genes or >6000 genes detected and >0.1 fraction of mitochondrial gene. Samples were normalized individually and integrated with the IntegrateData function. The integrated Seurat object was further scaled by regressing out UMI count and fraction of mitochondrial genes. Optimal principal components used for dimensionality reduction was determined empirically for each analysis by the drop off in PC variance. Cell cycle regression was not performed given small proliferating cell clusters identified in this study. Differential gene expression was analyzed using Seurat ‘FindAllMarkers’ or ‘FindMarker’ functions.

### Cell Type Annotation

We first used an automatic cell type annotation R package SingleR²³ to annotate cell types. Briefly, this algorithm computes the spearman correlation between the transcriptome of the test cell and reference data (i.e., bulk RNA-seq of a pure cell type or cell state) to define cell type label. The reference datasets used in this study include Human Primary Cell Atlas (HPCA) and Blueprint-Encode. We then manually refined cell type annotation based on marker genes identified using unsupervised clustering and differential expression analyses³⁴. All cell type marker genes used in this study were from literature.

### Tumor Cell Population Analysis

Tumor cells were identified as expressing at least two of the five literature reported marker genes²⁵⁻³⁰ above median value across all mesenchymal cells with non-zero values. Clustering and tumor cell state annotation were performed using normalized raw data. Tumor stemness score was calculated using Seurat AddModuleScore function based on relative expression of 50 tumor stem cell marker genes described previously⁵⁷.

### T Cell Population Analysis

Four AML tumors and paired normal kidneys were analyzed for T cell function, two of which were SLS-dominant and two of which were IS-dominant. T cell population was downsampled to have equal number of cells from SLS or IS samples. We calculated an exhaustion score for each cell based on relative expression of known checkpoint genes, including TIGIT, LAG3, and KLRG1; and a cytotoxic score based on relative expression of cytotoxic effectors, including GZMB, IFNG and TNF. Cells with expression of at least one checkpoint gene or one cytotoxic effector gene were calculated for the scores and were regarded as exhausted or cytotoxic respectively.

### Single Cell T Cell Receptor and T Cell Clonotype Analysis

Raw FASTQ reads were mapped to human GRCh38 V (D) J reference genome (v3.1.0, 10× Genomics) using Cell Ranger pipeline (10× Genomics). Sequencing depth was on average 20,876 reads/cell. The filtered contig annotation file was used for downstream analysis that contains high-confident contigs. For clonotype analysis, we downsampled to roughly equal number of cells derived from SLS and IS tumors. After normalizing the cell numbers, we detected 4,667 unique clonotypes in two IS-dominant tumors and 220 unique clonotypes in two SLS-dominant tumors. Clonotype size ranged from 1 to 632 cells in IS-dominant tumors and 1 to 23 cells in SLS-dominant tumors. We further defined clonotype expansion as that a clonotype shared by at least three cells within individual sample, and clonotype sharing as that a clonotype detected in any two or more T cell subtypes within individual sample. we detected that 69% of clonotypes were expanded in IS-dominant tumors, and 18% clonotypes were expended in SLS-dominant tumors. In IS-dominant tumors, we identified 229 and 319 shared clonotypes in CD8+ and CD4+ T cells respectively, whereas, in SLS-dominant tumors, we identified 5 and 0 shared clonotypes in CD8+ and CD4+ T cells respectively. One-sided Fisher's exact test followed by Benjamini-Hochberg correction was used to assess statistical significance of clonotype sharing among T cell subtypes on cluster-by-cluster contingency tables.

### RNA Velocity Analysis

RNA velocity was calculated using scVelo (v0.2.2, python package)⁸⁵ to infer the differentiation trajectory directionality and future cell state from ratio of unspliced and spliced mRNAs within a single cell. Individual loom file was generated for each sample based on Cell Ranger output file using velocity python package¹¹⁵ Then loom files were merged together for SLS samples and IS samples respectively. For visualization, we used Seurat generated single cell UMAP coordinates to project RNA velocity vectors onto the two-dimension embeddings.

### Regulon and Pathway Analysis

Transcription factor enrichment and regulon activity were assessed using SCENIC package⁴⁵ and human cisTarget databases: hg19-500 bp-upstream-7species.mc9nr.feather and hg19-tss-centered-10 kb-7species.mc9nr.feather. Seurat normalized expression matrix was used as input. Only the protein coding genes were analyzed for motif enrichment. We used GSVA (version 1.36.3) for pathway enrichment analysis with default parameters. The database used was Hallmark Gene Set from Molecular Signatures Database (MsigDB)¹¹⁶.

### Visium Spatial Transcriptomics Data Processing

Raw FASTQ files were aligned to human GRCh38 reference genome using Space Ranger pipeline (10× Genomics). Raw data were processed using Seurat (v4.0.2) for normalization using SCTranform function. Custom scripts were used to map normalized spot-level data to histology images for visualization. Tumor enriched spots were identified as spots with averaged value of CTSK and PMEL higher than median of average value across all spots. We then calculated scores for SLS and IS cell states on tumor-enriched spots using the marker genes MGP (for SLS) and ACTA2 (for IS) as these two marker genes were identified as most robust markers for these two distinct cell types in scRNA-Seq data. We used a stringent criterion to annotate SLS cell state: spots with value of MGP (SLS marker) higher than 75% across all spots and value of ACTA2 (IS marker) lower than 75% across all spots. Vice versa, IS state was identified as spots with value of ACTA2 (IS marker) higher than 75% across all spots and value of MGP (SLS marker) lower than 75% across all spots. SLS enriched island or IS enriched island were identified as island that only contain SLS state or IS state tumor enriched spots. Since the expression levels of TREM2 and TYROBP were within similar range, values of these two genes were simply averaged for each spot and plotted.

### Spatial Correlation Analysis

For the major cell types identified in scRNA-seq analysis (Tumor cells, T cells, B cells, macrophages, lymphatic endothelial cells, blood endothelial cells, NK cells and dendritic cells), we re-defined cell type marker genes with the criteria that the relative average expression is 3 times higher than any other cell types and expressed in at least 50% of cells of the given cell type. The Nanostring spatial transcriptomics data were Q3 normalized and log 2 transformed. Then, we calculated the relative frequency of each cell type in the Nanostring spatial transcriptomics datasets (12 bulk RNA-seq of ROIs of SLS tumor, and 12 bulk RNA-seq ROIs of IS tumor) by the average expression of scRNA-Seq re-defined cell type marker genes. To identify genes that may mediate cell-cell interactions, we performed Pearson correlation analysis of expression of genes that are primarily expressed in one cell type in the single cell data with the predicted frequency/activity of another cell type in the Nanostring spatial transcriptomics data of each ROI, followed by correlation test for significance assessment of the correlation coefficient. The assumption is that if a gene is highly expressed in one cell type and highly correlated with frequency/activity of another cell type, the given gene may mediate the interaction of these two cell types as previously described⁹⁰.

### Ligand-Receptor Interaction Analysis

Ligand-receptor interaction analysis was performed to infer potential cell-cell interactions via direct ligand-receptor binding using algorithm described previously⁸⁶ (https://github.mit.edu/mkumar/scRNAseq_communication). The set of ligand-receptor pairs were obtained from previous study¹¹⁷. We manually added more ligand-receptor pairs discovered more recently, including immune checkpoints and innate immune regulation. Briefly, interaction score of given ligand-receptor interaction between two cell types was calculated as the product of average ligand expression across all cells of one cell type and the average receptor expression across all cells of another cell types as previously described⁸⁶. We calculated average expression of ligand and receptor in all cell types using normalized expression data of the aggregated scRNA-Seq dataset. The statistical significance of pairs of interaction was determined by one-sided Wilcoxon rank-sum test.

### Kinetic Modeling of Purine Metabolic Pathway

As previously described⁶⁰, we employed a kinetic model of purine metabolism⁵⁹ that has the format of a Generalized Mass Action (GMA) system, where all processes are represented as products of power-law functions. The model contains 16 metabolites and 37 fluxes and a large number of regulatory signals⁶⁰. The diagram of the model structure was drawn using custom scripts. We generated pseudo-bulk expression data from scRNA-Seq data by averaging expression of each gene across all non-zero cells in a given cell type. We used the differential expression of each gene in the tumor cells compared to matched normal mesenchymal cells as a corresponding change in enzyme amount. The enzyme activities were lumped into apparent rate constants in the original model formulation. Therefore, the differential expression of each gene was modeled as a corresponding change in its respective reaction rate constant parameter. All other parameters were retained the same as at the original steady state. The equations of the model were then integrated to get a new steady state where the variable concentrations and fluxes of the system were studied.

### Bulk RNA-Seq Analysis

Bulk RNA-seq data were download from dbGaP (phs001357.v1.p1), including 10 TSC samples and 4 healthy controls. Genes with missing data in more than 5 samples were removed from downstream analysis. Raw data were log 2 normalized.

### Data Availability

The scRNA-seq data of 5 LAM samples, 6 AML and 4 matched normal kidney samples that support the findings of this study are available in GEO (GSE190260). The scTCR-seq data of 4 tumors and 10×Genomics spatial transcriptomics data are available in GEO (GSE208262). Nanostring data are available in GEO. The publicly available bulk RNA-seq data from TSC patients⁸ used in this study are available in the Database of Genotypes and Phenotypes (dbGaP) under the accession code phs001357.v1.p1. GRCh38 reference genome.

### Example 1. Single Cell Analysis of Angiomyolipomas (AML) and Lymphangioleiomyomatosis (LAM)

AML and LAM are hallmark manifestations of TSC3, and are also seen sporadically in patients without TSC. Six renal AML tumors and four matched normal tissues (Table A) obtained at the time of tumor resection were assessed with scRNA-Seq and paired scTCR-Seq using the 10× Chromium single cell 5′ chemistry (FIG. 1a). Five LAM lungs (table A) obtained at lung transplantation were also analyzed with scRNA-Seq. After filtering out low-quality cells, a total of 108,071 cells from the AML and 33,136 cells from the matched normal kidneys were analyzed; 42,202 cells were analyzed from the LAM lung samples.

TABLE A
Mutations identified in AML tumors. Genetic mutations
identified in each AML tumor are listed.

| AML ID | mutation |
|---|---|
| AML1098 | TSC1 c.1798C > T p.Q600* at 79% |
| AML1162 | TSC2 c2974 C > T (p.Q992*), exon 26 - in 41.33% of 1196 reads; |
| | TSC2 c5226 indels ( ), exon 40 - in 12.86% of 916 reads |
| AML1166 | TSC2 c.4537G > T (p.E1513*), exon 35 - in 35% of 343 reads; |
| | TSC2 c.4442dup (p.S1482Efs*42), exon 34 - in 36% of 297 reads |
| AML1167 | TSC2 c.1372C > T (p.R458*), exon 13 - in 47.56% of 1214 reads |
| AML1171 | TSC2 c.1111_1119 + 1delinsTC ( ), exon 11 - in 19% of 146 reads |
| AML1172 | TSC1 c.2074C > T (p.R692*), exon 17 - in 12% of 316 reads; TSC1 |
| | c.989_1011dup (p.I338*), exon 10 - in 4% of 304 reads |

Data integration identified tumor cells and all other expected major cell types in the immune and stromal compartments of AML tumors and matched normal tissues (FIG. 1b-c). Cell types were annotated first by unbiased cross-referencing to two databases of pure cell types, using SingleR²³, with normalized data. This was followed by manual annotation with cell type specific marker genes to refine cell type identification. AML and LAM cells were identified using a panel of five well-established marker genes known from prior work to be highly expressed in both AML and LAM²⁴ (CTSK²⁵, PMEL²⁶, VEGFD^{27,28}, MITF²⁹, and MLANA³⁰). Graph-based clustering was performed on the mesenchymal cell population using Seurat, resulting in eight clusters. Cells expressing at least two of the five marker genes at or above median expression across all mesenchymal cells with non-zero values were identified as AML/LAM cells. We observed that nearly all cells meeting this criterion were in three clusters (clusters 1, 2 and 6), and therefore, we annotated all cells in these three clusters as tumor cells. The number of AML cells (6%) and LAM cells (0.66%) was low. Importantly, no cells with this expression pattern were observed in the normal kidney specimens, strongly suggesting that this method of tumor cell identification was specific, although it may have undercounted the tumor cell fraction in both AML and LAM. Cells from each patient sample contributed to each cluster, suggesting an absence of major batch effects. Normal kidney contained 49% epithelial cells in contrast to 1.1% epithelial cells in AML, as expected (FIG. 1d). Many immune populations were enriched in tumors compared to matched normal, including macrophages (18.3% vs 2.7%), dendritic cells (4% vs 0.7%), and T cells (32.6% vs 14.1%). We also identified proliferating T cells and proliferating macrophages in AML.

The major cell types identified in LAM lung included immune cells (T cells, NK cells, B cells, macrophages and monocytes), mesenchymal cells, epithelial cells and endothelial cells (lymphatic and blood) (FIG. 1e). Proliferating macrophages were also identified in LAM. In contrast to the AML, no proliferating T lymphocytes were identified in the LAM specimens.

### Example 2. Global Mapping of Pathways and Genetic Regulatory Networks in AML Cells

Re-clustering of mesenchymal population showed separate clusters of cells from normal kidneys and AML tumors (FIG. 1f). The trimodal cluster consists of cells derived solely from AML tumors. Besides AML cells (as described above), the cluster also contains tumor associated fibroblasts (TAF) with no expression of tumor marker genes but high expression of known TAF marker genes: Tumor-Derived Adhesion Factor (IGFBP7)³¹, Fibroblast-Specific Protein-1 (FSP1/S100A4)³², Platelet-Derived Growth Factor Receptor Beta (PDGFRB)³², Secreted Protein Acidic And Rich in Cysteine (SPARC), and SPARC-Like Protein 1 (SPARCL1) (FIG. 1f). TAF have been shown to promote tumor proliferation in many human cancers³³.

Differential gene expression analysis by Seurat^{34,35} identified 160 genes uniquely upregulated in tumor cells compared with TAF and normal kidney, including genes previously reported (e.g., GPNMB⁸, SQSTM1/p62³⁶, MMP 2³⁷, PTGDS³⁸) and genes involved in tumor metastasis (e.g. MMP11, MDK, DCN, PDPN) (FIG. 1g). Two long non-coding RNAs (lncRNAs) (MALATI, NEAT1) were upregulated in both tumor cells and tumor-associated fibroblasts compared to matched normal mesenchymal cells (FIG. 1g), suggesting remodeling of fibroblasts by AML cells.

To identify pathways differentially regulated in AML cells vs. TAF and normal kidney, we used Gene Set Variation Analysis (GSVA)³⁹, a non-parametric, unsupervised method for estimating variation of gene set enrichment. Hallmark gene set analysis (containing 50 gene sets) identified genes involved in cholesterol homeostasis as the most upregulated pathway in AML cells, consistent with prior work^{40,41}, while the second most upregulated pathway was mTORC1 signaling, a well-known biochemical effect of TSC2 loss in AMLs and LAM (FIG. 1h). ROS, glycolysis, and adipogenesis pathways were also enriched in AML, consistent with prior work^{36,42-44}

To investigate transcriptional networks driving the expression characteristics of AML, we used Single-Cell Regulatory Network Inference and Clustering (SCENIC)⁴⁵. This regulon analysis revealed that more regulons were upregulated in AML cells rather than downregulated. Known TSC-associated transcription factors^{9,46-48} were re-identified, such as MITF and TFE3, for which both expression and regulon activity were much higher in tumor cells compared to normal kidney mesenchymal cells. Similarly, SREBF1/SREBF2 and PPARG, known master regulators of lipid and cholesterol metabolism downstream of mTORC140,41,49, had both high expression and high regulon activities in AML cells. This analysis also identified transcription factors and regulons associated with AML, including several involved in epigenetic regulation, e.g. HDAC2, SIRT6, FOXN3, MEF2A (FIG. 1i).

Specific genes of interest include MDK (newly identified here as highly expressed in AML) and GPNMB (a known marker of AML⁵⁰), both of which are increased in AML cells in both the scRNA-Seq dataset (FIG. 1g) and the bulk RNA-seq dataset of tumor samples⁸. Using RNA in situ hybridization, MDK expression was detected in AML tumors but not in adjacent normal kidney (FIG. 1j), consistent with the single cell data. MDK is a direct target of the transcription factor SP 1⁵¹, and regulon analysis showed enriched SPI expression and activity in the cells with high MDK expression. AML cells with high MDK expression showed higher expression of HIF1A, which binds to a hypoxia responsive element in the MDK promoter⁵². MDK is an heparin-binding growth factor⁵³ that promotes cell growth and angiogenesis^{54,55}.

Based on an evaluation of data published in Giannikou et al., Modern Pathology 2021 34 (2): 264-279, MDK RNA levels were seen to be 55-fold higher in Subependymal giant cell astrocytoma in comparison to normal brain. However, MDK RNA levels were not significantly different in Subependymal giant cell astrocytoma in comparison to other types of brain tumors.

### Example 3. AML Tumor Cells Exhibit Two Major States: Stem-Like and Inflammatory

UMAP re-clustering of the 6,596 AML cells revealed four clusters (FIG. 2a-b). Differential expression analysis revealed high expression of modulated smooth muscle genes⁵⁶ in cluster 1 and high expression of contractile smooth muscle genes⁵⁶ in cluster 2. Clusters 0 and 3 appeared to represent intermediate or transitional cell states between clusters 1 and 2, with a gradient expression of modulated and contractile smooth muscle marker genes. Cluster 1 showed relatively high expression of the mesoderm-specific transcription factor 21 (TCF21), a master regulator of phenotypic modulation of smooth muscle cells⁵⁶ (FIG. 2b). In disease conditions, phenotypic modulation transforms smooth muscle cells from a differentiated contractile state into a dedifferentiated modulated state. We noticed that several genes (SOX4, TCF4) (FIG. 2b), known to be stem cell markers, were upregulated in cluster 1, and therefore calculated the “stemness score” using a curated list of 50 tumor stemness marker genes⁵⁷. Cluster 1 showed the highest stemness scores which declined in a gradient leading to cluster 2 (FIG. 2c), as well as high activity of signaling pathways involved in stemness including Notch, Hedgehog, and WNT pathways. Cluster 2 was enriched in immune pathways, and showed high expression of inflammatory genes including CCL3, CCL4 and IL1B (FIG. 2b). Based on these features, we defined cluster 1 as a stem-like state (SLS) and cluster 2 as an inflammatory state (IS). Differential expression analyses of cluster 1 (SLS) versus cluster 2 (IS) identified 231 differentially expressed genes at fold change >2. Metabolic kinetic models using generalized mass action (GMA) equations have been used to simulate and predict biological processes^{58,59}. We previously showed that kinetic models of metabolic pathway systems can be used to interpret transcriptomic profiles measured during disease for cellular metabolism modeling⁶⁰. Purine related metabolism is linked to the mTORC1 pathway⁶¹⁻⁶³, and high levels of purine nucleotides are required to maintain cancer stemness⁶⁴, while external hypoxanthine supplementation promotes tumor stemness⁶⁴. Therefore, we generated pseudo-bulk RNA-seq data from single cell transcriptomes to infer cellular purine metabolism in both SLS and IS populations as well as normal mesenchymal cells obtained from matched normal kidney in this study. We found that metabolism of guanine/guanosine in the purine pathway was elevated in both tumor cell states compared to normal controls (FIG. 2d). In contrast, hypoxanthine and inosine metabolism were elevated specifically in the SLS population, suggesting that metabolic mechanisms may contribute to the high stemness features seen in the population.

The SLS (cluster 1) also showed higher expression of genes associated with TGF-beta signaling and the hypoxia pathway (two main triggers of tumor cell dormancy)^{14,65}. It has been increasingly recognized that a hypoxic microenvironment, as well as stress induced during metastasis, trigger a dormant state in which tumor cells become resistant to drug treatment and stress⁶⁶. Further analysis of a panel of dormancy marker genes revealed high expression in the SLS population (cluster 1), including the transcription factor NR2F1 (FIG. 2e). NR2F1 serves as a critical node in the induction and maintenance of tumor stem cell dormancy by integrating epigenetic programs of quiescence and survival^{14,67}. Regulon analysis confirmed that NR2F1 regulon activity (pathway activity of 41 genes regulated by NR2F1) was upregulated in the SLS (cluster 1) (FIG. 2f).

Other dormancy marker genes also showed high expression in SLS, including DEC2 (BHLHE41), Hypoxia Inducible Factor 1 Subunit Alpha (HIF1A), and estrogen receptor alpha (ESRI) (FIG. 2e). Estrogen receptor alpha was shown to be required by breast cancer cells to enter NR2F1-dependent dormancy¹⁴. Hormonal signaling is of particular interest in TSC, since 1) LAM affects almost exclusively women, 2) LAM and AML cells express ER alpha, and 3) estrogen impacts the survival, metastasis, and metabolism of TSC2-deficient cells in models of LAM⁶⁸. To investigate whether ER alpha contributes to dormancy in TSC-deficient settings, as suggested by the scRNA-Seq data, we used TSC2-deficient 621-101 cells⁶⁹, which were derived from a LAM patient's angiomyolipoma. The cells were treated with 100 nM estradiol or vehicle control for 24 hours and subjected to scRNA-Seq. All of the major dormancy genes were upregulated in the estradiol treated group compared to the control group. The related gene Estrogen Related Receptor Alpha (ESRRA) was also elevated by estradiol treatment. Regulon analysis further showed that estradiol treatment increased NR2F1 expression and regulon activity (FIG. 2g).

The identification of SLS and IS populations was validated in tumor specimens by co-staining with antibodies to SLS and IS markers (MDK and TAGLN respectively, FIG. 2i), and Cathepsin K (AML/LAM marker gene²⁵). MDK positivity was observed primarily in one population, while TAGLN positivity was observed primarily in a separate population (FIG. 2i). As expected, CTSK stained both populations. Quantification revealed little co-localization of MDK and TAGLN, versus extensive co-staining of MDK with CTSK or TAGLN with CTSK (FIG. 2j), supporting the existence of two distinct populations of AML cells, MDK⁺ and TAGLN⁺.

### Example 4. Cell Populations Occur in LAM that are Similar to the Two Types Observed in AML

In the sporadic form of LAM, angiomyolipoma are common, and genetic studies have shown that the AML and LAM cells arise from a common precursor cell⁷⁰. To determine whether the two cell states identified in AML are present in pulmonary LAM, we analyzed 57,186 cells from five LAM lungs using the same marker gene set and method as used for AML. A total of 375 LAM cells were identified (FIG. 1e). Considering the LAM cells alone, clustering revealed four clusters (FIG. 2k). Similar to AML, one cluster expressed SLS/modulated smooth muscle marker genes (cluster 2) and another cluster expressed IS/contractile smooth muscle marker genes (cluster 1) (FIG. 2l). An intermediate state with expression of all these genes was also identified (cluster 0). The SLS population and the intermediate state showed higher stemness score (FIG. 2m) and genes associated with dormancy were upregulated in the SLS population and intermediate state, similar to the SLS cluster in AML. In addition, like the SLS AML cells, the SLS cluster of LAM cells had upregulation of NF2F1 expression and regulon activity (FIG. 2n). Interestingly, the expression of VEGFD, a validated LAM biomarker⁷¹, was much lower than MDK (a potent angiogenic and lymphangiogenic growth factor^{55,72}) in LAM cells (FIG. 20), suggesting a potential role of MDK in LAM-associated lymphangiogenesis. Thus, we measured MDK serum levels in women with LAM and healthy controls and found that MDK levels were 3.7-fold higher in LAM patients (n=20) compared to healthy controls (n=19) (p=0.0361, FIG. 2p).

### Example 5. The Stem-Like Population of AML Cells May Contribute to Rapamycin Resistance

Rapalog therapy for AML and LAM leads to sustained but incomplete responses, with regrowth of AML and ongoing loss of lung function in LAM when treatment is stopped^{6,7}. These partial responses suggest possible drug tolerance in a subset of AML/LAM tumor cells. Our observation of elevated stemness and dormancy in a subset of tumor cells, typical features of drug-tolerant tumor persister cells⁷³, led us to directly examine rapamycin tolerance in AML cells. We developed a primary culture from one of the AML tumors profiled in this study (AML1162 with TSC2 mutation allele frequency of 41%). After one week in culture, these cells were treated with either DMSO (control) or rapamycin for 24 hours followed by scRNA-Seq profiling. A total of 2,066 cells and 4,083 cells were analyzed in the control and treatment group, respectively, after filtering out low quality cells (FIG. 3a). Merging these two sets of cells, UMAP clustering identified seven clusters (FIG. 3b). Using the same expression criteria described above, a total of 2004 candidate AML cells were identified, accounting for 33% of all cells (FIG. 3c).

Rapamycin had a striking effect on overall transcriptomes, and most clusters were composed nearly entirely of either treated or untreated cells. Strikingly, we identified a small cluster (cluster 4) that contained AML cells from both control and rapamycin treatment groups, suggesting that it contained cells that are resistant to rapamycin, or at least cells in which transcription was not changed by rapamycin treatment. In this cluster, the expression of many AML tumor marker genes was unaffected by rapamycin, in contrast to other clusters where rapamycin suppressed expression of these tumor genes (FIG. 3L), including CTSK (FIG. 3d).

Further analysis of cluster 4 showed high expression of tumor marker genes (FIG. 3M, only DMSO control group is shown in the UMAP), with a strikingly similar expression pattern to that of the SLS population of AML tumors. For instance, we have identified elevated expression of SOX4, PTGDS, MMP2 among other marker genes in AML tumors, suggesting that cluster 4 corresponds to the SLS state of AML cells. In addition, cells in the cluster 4 showed a high stemness score (FIG. 3e), and high dormancy score (calculated by expression of known dormancy marker genes¹⁴ including the dormancy inducer NR2F1 and hormonal regulator ESRI) (FIG. 3f and FIG. 3n). Consistent with these results, NR2F1 regulon activity was high in this cluster (FIG. 3g).

Levels of the dormancy inducer NR2F1 and the hormonal regulator ESRI (ERa) were unchanged by rapamycin (FIG. 3L), suggesting that dormancy may be associated with treatment resistance. Expression of SQSTM1 (p62) and SOD2, which help to maintain cellular ROS homeostasis in TSC36, were unaffected by rapamycin in cluster 4 (FIG. 3L), suggesting that redox homeostasis maintenance may be involved in treatment tolerance. These data suggest that the SLS state is resistant to rapamycin treatment, which is consistent with the notion that acquired stemness and dormancy render tumor cells resistant to chemical therapeutics⁷⁴⁻⁷⁶

MDK is reported to mediate drug resistance in other tumors⁷⁷, and we observed high expression of MDK in the SLS population (FIG. 2h). To determine whether MDK is involved in rapamycin tolerance and whether MDK is regulated by TSC pathway, we used two cellular models of TSC and found that expression of MDK was upregulated in TSC2-deficient AML patient-derived 621-101 cells compared to TSC2-reexpressing 621-103 cells, as well as in mouse kidney derived TSC2-deficient TTJ cells⁷⁸ compared to TSC2-addback TTJ+TSC2 cells (FIG. 3h). Because MDK is a secreted cytokine, we further assessed MDK protein levels in the cell culture medium by ELISA. MDK levels were significantly higher in both the patient-derived and mouse-derived TSC2-deficient cell lines compared with TSC2-addback controls (FIG. 3i).

Next, to assess the importance of MDK expression on rapamycin resistance in vitro and in vivo, we used an MDK inhibitor (iMDK) that specifically inhibits MDK but not other growth factors such as VEGF or pleiotrophin (PTN) (homologous to MDK)⁷⁹ and was shown to potently inhibit MDK and thus enhance PD-1 therapy in melanoma mouse models⁸⁰. TSC2-deficient cells (621-101, TTJ) and normal human fibroblasts (NHLF) were treated with DMSO, rapamycin (20 nM), iMDK (1 μM), or a combination of rapamycin (20 nM) and iMDK (1 μM). Treatment with iMDK alone had minimal effects in all 3 cell lines. However, when combined with rapamycin, iMDK had a synergistic effect on the two TSC2-null cell lines (FIG. 3j). We defined synergy as the combined effect of two drugs is greater than the sum of each drug's individual activity^{81,82}. In normal fibroblasts (NHLF), rapamycin had a dramatic growth inhibitory effect, which was not significantly changed by the addition of iMDK. To determine whether iMDK sensitizes tumors to rapamycin treatment in vivo, we generated subcutaneous tumors using the TSC2-deficient TTJ cells in immune-deficient athymic nude mice. Combination treatment with iMDK and rapamycin led to a more rapid onset of tumor response, and a lower tumor burden, compared with rapamycin alone, while iMDK alone had no apparent effect (FIG. 3k, FIG. 3O). Many cancers have relatively high MDK expression in comparison to matched normal tissues, including bladder cancer (FIG. 3P). We found that three bladder cancer cell lines were also sensitized by iMDK to rapamycin treatment (FIG. 3Q). These data may provide a rationale for combination therapy targeting MDK and mTORC1 in TSC and other selected tumors.

### Example 6. Remodeling of Endothelial Cells by Heterogeneous Tumor Cell States

We next investigated the potential differential effects of these two cell states, SLS and IS, on the tumor microenvironment. As seen in FIGS. 1A-J, both blood and lymphatic endothelial cells were enriched in AML compared with adjacent normal kidney, suggesting ongoing angiogenesis and lymphangiogenesis. Strikingly, the distribution of IS and SLS was not uniform among our six AML samples, with two AMLs consisting mainly of IS (>70%), and four mainly SLS (>80%) (FIG. 4a). The SLS-dominant tumors had a much greater content of endothelial cells, with an average of 24.9% fenestrated endothelial cells and 1% lymphatic endothelial cells, in contrast to the IS-dominant tumors with average 1.4% fenestrated endothelial cells and 0.6% lymphatic endothelial cells (FIG. 4b). To validate this, immunohistochemistry (IHC) staining for the endothelial marker CD31 was performed on each AML and the percentage of endothelial cells was calculated by digital analysis, revealing a strong correlation with the percentage predicted by scRNA-Seq (FIG. 4c) and a higher percentage of endothelial cells in SLS-dominant tumors (FIG. 4d). This dramatic difference in the endothelial composition in SLS-dominant vs. IS-dominant tumors suggests that the endothelial cells are responding to specific cues arising from the predominant cell type within the tumor. Thus, we investigated all genes that were over-expressed in SLS compared to IS and again identified MDK as the top differentially expressed angiogenic gene (by fold change). VEGF-D (a pro-lymphangiogenic factor) is thought to drive pulmonary lymphangiogenesis in LAM. Differential expression analysis showed much higher expression of MDK than VEGFD in general, with higher expression of MDK in cluster 1 (SLS) and higher expression of VEGFD in cluster 3 (IS) (FIG. 4e). Interestingly, VEGFA, another well-recognized pro-angiogenic factor was only expressed in a small number of SLS cells. Taken together, these data suggest that high expression of pro-angiogenic MDK in SLS tumors may account for the enriched endothelial cells in this subtype of AML.

Further differential and pathway analysis revealed remodeling of endothelial cells in AML, including high expression of C-C Motif Chemokine Ligand 21 (CCL21), TBX1 and NRP2 specifically observed in tumor LECs. Regulon analysis further revealed that transcription factors NR2F1 and NR2F2 may underlie these transcriptional programs.

### Example 7. T Cell Dysfunction and Suppressed Clonal Expansion in SLS-Dominant Tumors Revealed by Integrative Analysis of scRNA-Seq and scTCR-Seq

T cell infiltration and exhaustion have been observed in human TSC tumors, and a clear benefit of immunotherapy was observed in mouse models^{17,18}. To determine whether T cells are influenced by tumor cell states in AML, we focused on the four AMLs with paired normal kidneys profiled, two of which were SLS-dominant and two of which were IS-dominant. Pathway activity analysis of tumor-derived T cells compared to that from paired normal kidneys revealed upregulation of inflammatory responses, including the type I and type II interferon pathways. Cell proliferation pathways (E2F targets, MYC targets, Mitotic signaling) were also consistently upregulated in tumor-derived T lymphocytes, in line with the observed general expansion of T cells (FIG. 1d). A population of proliferating CD8+ cells (CD8 T-prolif) was present specifically in the tumors and not in normal kidney, suggesting an expansion of tumor antigen-reactive T lymphocytes. T cell expansion in tumors was confirmed by CD3 IHC. Multiple immune checkpoint markers were expressed in tumor derived T cells.

The higher fraction of T cells in IS-dominant tumors compared to SLS-dominant tumors suggests more T cell infiltration and/or T cell proliferation in IS-dominant tumors, consistent with previous reports that stem-like states in tumors are associated with immunoresistance⁸³. Re-clustering of tumor-derived CD8+ T cells (down-sampled to have equal number of cells from SLS or IS samples) revealed three major clusters: memory/naïve T cells (CD8 Tm/naïve), effector T cells (CD8 Teff) and proliferating T cells (CD8 T-prolif), as well as subclusters within each major cluster, with different expression of immune checkpoint genes or cytotoxic effector genes (FIG. 5a-b). We calculated an exhaustion score for each cell based on relative expression of known checkpoint genes, including T Cell Immunoreceptor With Ig And ITIM Domains (TIGIT), Lymphocyte Activating 3 (LAG3), B- and T-Lymphocyte Attenuator (BTLA) and Killer Cell Lectin Like Receptor G1 (KLRG1); and a cytotoxic score based on relative expression of cytotoxic effectors, including Granzyme B (GZMB), Interferon Gamma (IFNG) and Tumor Necrosis Factor (TNF). CD8+ T cells derived from SLS-dominant tumors showed much lower cytotoxic scores compared to those derived from IS-dominant tumors, and a lower percentage of cytotoxic cells (defined as expressing at least one cytotoxic effector genes) within each subpopulation (FIG. 5c). In addition, SLS-dominant tumor derived cells exhibited higher exhaustion scores (FIG. 5c). Despite a roughly equal frequency of exhausted cells in each subpopulation (FIG. 5c), the fraction of exhausted CD8+ Teff cells in SLS-dominant tumors was higher than that in IS-dominant tumors, and IS-dominant tumors showed a higher frequency of both cytotoxic CD8+ Teff and CD8+Tm/Naïve populations (FIG. 5d). Similar analysis of tumor-derived CD4+ T cells revealed six subtypes of CD4+ T cells (FIG. 5e-f). While memory CD4+ T cells and CD40LG-high population derived from IS-dominant tumors showed a higher cytotoxicity score (FIG. 5g), no significant difference in cell frequency in any subtype was observed between SLS-dominant and IS-dominant tumors (FIG. 5h).

Tumor activated lymphocytes undergo clonal expansion, and expanded T cells from the same clone have the same TCR sequence (clonotypes), which enables tracking of differentiation trajectories. We examined sharing of expanded TCR clonotypes across all sub-populations of CD8+ and CD4+ T cells within individual samples after cell number normalization, which revealed 229 clonotypes shared among CD8+ T cell subtypes and 319 clonotypes shared among CD4+ T cell subtypes in IS-dominant tumors but only 5 clonotypes shared among CD8+T subtypes in SLS-dominant tumors (FIG. 5i). While SLS tumors showed quite limited clonotype sharing among subtypes in CD8+T population and no clonotype sharing in CD4+T population, IS tumors exhibited extensive clonotype sharing among subtypes in both CD8+T and CD4+T populations (FIG. 5j): 75% of expanded TCRs in the CD8+ Teff subtype were shared with the CD8+Tm/Naïve subtype in IS tumors, revealing a dynamic connection between these two CD8+ T cell states. In IS tumors, the majority of proliferating CD8+ T cells shared clonotypes with CD8+ Teff population, which may imply a tumor antigen-reactive T cell proliferation (FIG. 5j). Proliferating T cells shared a high number of clonotypes with two cytotoxic Teff populations (CD8 Teff-TNF and CD8 Teff-IFNG). In addition, extensive clonal sharing was observed between CD8 Teff and two cytotoxic Teff populations (CD8 Teff-TNF and CD8 Teff-IFNG), suggesting an active and dynamic differentiation trajectory toward functional T cells. These observations suggest that the high frequency of cytotoxic CD8+ T cells observed in IS tumors is at least partially due to a dynamic differentiation of presumably tumor-recognizing effector cells. As expected based on previous work⁸⁴, CD4+ T cells showed less clonal expansion in general compared to CD8+T population. CD4+ T cells clonal sharing was only detected in IS-dominant tumors. The Tregs cluster shared TCRs with CD4 T-cluster1, CD4 Tm and CD4 T-CTLA4 clusters (FIG. 5j), suggesting a complex dynamic differentiation of Tregs in tumors.

To infer dynamic differentiation among subtype T cells, we calculated splicing-based RNA velocity using single cell transcriptome data⁸⁵. Consistent with the substantial clonal connectivity observed in IS-dominant tumors, this analysis supported a differentiation trajectory from CD8 effector T cells to proliferating T cells and from multiple CD4+T subpopulations to Tregs (FIG. 5k). In contrast, SLS-dominant tumors showed limited differentiation potential among subtypes.

Given the striking difference in T cell modulation in SLS versus IS dominant tumors, we next explored whether SLS tumor cells express higher levels of immune checkpoint genes to inhibit T cells. We analyzed TIGIT ligands (PVR, NECTIN2), BTLA ligand (TNFRSF14), LAG3 ligand (HLA-DRA, FGL1), KLRG1 ligand (CDH1, CDH2), and PD-1 ligands (CD274, PDCD1LG2). Surprisingly, all of these ligands showed low expression in both groups of tumor cells. The low expression levels and lack of significant differences of these immune checkpoint ligands between SLS versus IS tumors suggest other mechanisms in the differential modulation of T cell function in these tumor cell states.

### Example 8. Delineating the Suppressive Immune Microenvironment in TSC

Immunosuppressive myeloid cells, such as tumor-associated macrophages (TAMs), are considered major barriers to cancer immunotherapy⁸⁶, due to their potent suppressive function and high abundance in the tumor microenvironment⁸⁷. As noted above, enrichment of macrophages represented the most striking immune infiltration in AML (FIG. 1b,1d). This enrichment of macrophages in the AML was confirmed by CD68 IHC (FIG. 6a). These AML-derived macrophages showed higher expression of the immune checkpoint genes T cell immunoglobulin and mucin domain-containing protein 3 (TIM3) encoded by HAVCR2, and V-domain immunoglobulin suppressor of T cell activation (VISTA) encoded by VSIR, in comparison to macrophages derived from matched normal kidneys (FIG. 6b). The expression of other immune checkpoint genes is provided in FIG. 6N. Expression of VISTA and TIM3 on tumor infiltrating macrophages is associated with T cell dysfunction in the tumor microenvironment^{88,89}.

Tumor cells may influence other cells in the microenvironment by direct ligand-receptor interactions or indirect cell-to-cell communications in which tumor cells produce a signal (such as paracrine effectors) to recruit or exclude immune cells and alter their behavior^{86,90}. Therefore, we further analyzed one SLS tumor and one IS tumor using Nanostring digital spatial profiler (DSP) to query spatial tumor-microenvironment organization, and confirmed higher expression of the IS marker gene ACTA2 in IS-dominant tumors (FIG. 6c). For each tumor, we selected 12 regions of interest (ROIs) that were enriched with tumor cells (smooth muscle actin positive), T cells (CD3 positive), and macrophages (CD68 positive) for RNA sequencing. Since single cell data have much higher resolution, we re-defined gene signatures for each of the major cell types identified in AML, including macrophages, Langerhans cells, dendritic cells, T cells, B cells, lymphatic endothelial cells and blood endothelial cells. We then used these cell type specific gene signatures to deconvolute the cell composition of each selected area and to infer the relative frequency or activity of these cell types in each of the ROIs, and searched for genes primarily expressed by tumor cells that may influence or correlate with the frequency/activity of another cell type. We reasoned that genes expressed primarily by tumor cells may influence a different cell type in the tumor microenvironment by an indirect paracrine signal, hence correlation analysis of the expression of genes (primarily expressed in tumor cells) and frequency/activity of another cell type in each ROI was performed to reveal genes mediating cell-to-cell communication, as shown previously⁹⁰. We found a high correlation of APOE, LGALSI and PCSKIN, which were primarily expressed in SLS tumor cells, with macrophage frequency/activity in SLS-dominant tumors (FIG. 6d). These correlations were not observed in IS-dominant tumors. Interestingly, all of these genes encode secreted proteins, suggesting a specific paracrine regulatory role of SLS tumor cell secretome on macrophages. Consistent with this concept, APOE and LGALS1 were previously shown to promote M2 polarization of macrophage/microglia in mouse models^{91,92}. We next analyzed a published bulk RNA-Seq dataset of ten AML tumors⁸, which again revealed a high correlation between APOE and macrophage population frequency. Since bulk RNA-Seq data are confounded by tumor purity and tumor heterogeneity, the robust identification of this correlation strongly supports the existence of a tumor-macrophage regulatory axis.

To search for putative macrophage receptors for these tumor ligands and to profile the full spectrum of ligand-receptor mediated direct tumor-microenvironment interactions, we next performed ligand-receptor interaction analysis using a validated algorithm previously described⁸⁶ and a list of over 2,500 curated pairs of ligand-receptors to infer putative tumor-microenvironment interaction based on ligand expression in one cell type and corresponding receptor expression in another cell type. This revealed tumor-macrophage interactions via APOE-TYROBP (DAP12) as the strongest interaction among tumor-microenvironment interactions (FIG. 6e). TYROBP and TREM2 form a receptor complex on macrophages which has been extensively studied in the context of neurodegenerative diseases, where the complex mediates signaling and cell activation following binding to its ligands including APOE or β-amyloid (a cleavage product of the amyloid-beta precursor protein APP) 93-9 Interestingly, APP also showed strong interaction with TYROBP. Recent studies have shown that TREM2+/TYROBP+ tumor-associated macrophages (TAMs) suppress T cell function and proliferation in various tumors and that targeting this TAM population can modulate immunosuppressive TAMs and restore T cell function^{96,97}.

To compare macrophages derived from SLS and IS tumors, we down sampled to 5,000 cells from each tumor type and included all macrophages derived from matched normal kidneys for downstream analysis. Re-clustering identified 12 clusters (FIG. 6f). Most clusters were primarily derived from tumors except two small clusters (cluster 8 and cluster 9) that were mainly derived from normal kidneys (FIG. 6g-h). Two main types of macrophages (tissue resident macrophages and TAMs) were identified in the tumor derived macrophage population. Cluster 2 and cluster 3 were annotated as tissue resident macrophages (TRM) based on high expression of IL7R⁹⁸ and inflammatory genes (FIG. 6i). The TAMs in AML are mainly composed of 4 clusters (cluster 0, cluster 1, cluster 4, and cluster 6) characterized by a high M2 module score, which was calculated by the relative expression of alternatively activated macrophage marker genes, including CD163⁹⁹, MRC1⁹⁹, VEGFA¹⁰⁰ and TREM2⁹⁶ (FIG. 6h). Surprisingly, the organization of TAMs showed a striking difference between SLS and IS tumors: cluster 1 and cluster 6 were mainly composed of cells derived from SLS tumors, whereas cluster 0 and cluster 4 were mainly composed of cells derived from IS tumors (FIG. 6i). Cells from cluster 1 and cluster 6 showed high expression of TREM2 and TYROBP (FIG. 6k). These data show that there is a higher percentage of TREM2+/TYROBP+ TAMs derived from SLS tumors.

These observations suggest a regulatory axis from SLS tumor cells to TAMs via an APOE-TREM2/TYROBP interaction, with APOE as a putative ligand for the TREM2/TYROBP complex in tumor TME. Consistent with this hypothesis, APOE (and APP) showed higher expression in SLS AML cells compared to IS cells (FIG. 6l). We sought to validate this observation using 10× Visium spatial transcriptomic profiling in an independent AML sample. We used CTSK and PMEL to identify AML cells (FIG. 6m). Spots with averaged expression of CTSK and PMEL higher than 50% across all spots were annotated as tumor spots. We then calculated scores for SLS and IS within identified tumor spots using the most robust marker genes MGP (for SLS) and ACTA2 (for IS) (see Methods), and identified islands enriched with SLS or IS. Plotting average expression of TREM2 and TYROBP (red) revealed higher expression in the SLS enriched island compared to IS enriched island (FIG. 6m). APOE also showed higher expression in the SLS enriched island (FIG. 6m).

Our discovery of a striking suppression of CD8+ T cells in SLS-dominant tumors is consistent with the reported role of TREM2+/TYROBOP+ TAMs in suppressing CD8+ T cell function and proliferation in tumors⁹⁶. The higher T cell clonal expansion and dynamic differentiation in IS-dominant tumors suggest tumor-reactive T cell activation. Taken together, this tumor-specific inhibition of T cell function and T cell proliferation/differentiation in SLS-dominant AML implies a major immunomodulatory role of myeloid cells in TSC. This has particular importance given the extremely low expression of immune checkpoint ligands on the AML tumor cells.

### Example 9. Analysis of Molecular Interactions Between Tumor and Tumor Microenvironment Provides Potential Targets for Distinct Precision Therapeutic Strategies for SLS and IS Tumor

In the immune compartment, we also observed enrichment of B lymphocytes and dendritic cells in AML relative to normal kidney. We detected 1,620 B cells predominately from tumor (FIG. 7a-b). Re-clustering revealed six clusters. Of these, five were particularly tumor enriched. We identified follicular B cells expressing high levels of CD20 (MS4A1) and CXCR5 in both tumor (cluster 5) and adjacent normal kidneys (clusters 1) (FIG. 7b). In contrast, plasma B cells expressing immunoglobulin gamma (IGHG1, CD27, CD38) were exclusively enriched in tumors (FIG. 7c). Pathway analysis identified induced interferon gamma and TGF beta signaling in regulatory B cells, suggesting a regulatory role of Tregs in tumor microenvironment¹⁰¹. A pattern of reduced activity in tumor-specific plasma B cells, evidenced by a universal downregulation of pathways involved in cell growth (Myc targets, mTOR pathway) and inflammation (interferon alpha/gamma, IL2 and TNF alpha signaling), may suggest reduced function of plasma cells in the tumors.

We detected 839 cross-presenting dendritic cells expressing CLEC9A and XCR1 exclusively in tumors. Re-clustering identified a small cluster of proliferating cells (cluster 3) (FIG. 7d-e). This cluster showed higher activity of Myc targets, E2M targets and mTORC1 signaling. HAVCR2 (TIM-3) has been reported to be an important regulatory factor of dendritic cells in anti-tumor immunity^{102,103}. The proliferating cluster exhibited high HAVCR2 expression (FIG. 7f), suggesting a pro-tumoral function of proliferating dendritic cells.

Although AML has an extremely low mutational burden¹⁹, the overall enrichment of plasma B cells and cross-presenting dendritic cells in tumors may suggest tumor-specific antigen presentation in tumor microenvironment that may include all the genes/proteins highly expressed in AML, including CTSK and MDK.

Tumor-microenvironment interactions play crucial roles in tumor development¹⁰⁴. To assess the comprehensive crosstalk between tumor and tumor microenvironment, we quantified potential cell-cell interactions among all cell types in the tumor microenvironment as described above. We observed numerous interactions in SLS-dominant tumors, including B2M-HLA-F, HLA-B-CANX, and MIF-CD74 in B and T cells, similar to what has been reported for melanoma⁸⁶, which is of interest because as described above, AML express many melanoma marker genes including MITF, PMEL and MLANA^{46,47}. Interestingly, we identified more tumor-TAF interactions in SLS-dominant tumors (21 pairs) compared to IS-dominant tumors (4 pairs) by ligand-receptor analysis. We also observed extensive interactions of KLRD1 and HLA family members between NK cells and other cell types, and interactions between tumor cells and other cell types related to extracellular matrix remodeling. Tumor cell secreted extracellular matrix molecule such as collagen (COL4A1) can bind to adhesion receptors broadly expressed on many cell types, such as integrin receptor ITGB1. We observed expression by tumor cells of thrombospondin (THBS1) and tissue inhibitors of metalloproteinases (TIMP1 and TIMP2), secreted factors involved in extracellular matrix remodeling.

Differential analysis of landscape of ligand-receptors interactions in SLS-dominant versus IS-dominant tumors revealed different tumor-microenvironment crosstalk in these two tumor cell states. For example, more interactions between tumor and blood endothelial cells were found in SLS-dominant tumors, consistent with enriched endothelial cells in SLS-dominant tumors. The depletion of interactions of CD8 and CD4 T cells with other cell types in SLS-dominant tumors may underlie the molecular mechanisms for the observed suppressed T cell clonal expansion.

### REFERENCE

1. Ben-Sahra I, Manning B D. mTORC1 signaling and the metabolic control of cell growth. Curr Opin Cell Biol. 2017; 45:72-82.
2. Sabatini D M. Twenty-five years of mTOR: Uncovering the link from nutrients to growth. Proc Natl Acad Sci USA. 2017; 114 (45): 11818-11825.
3 Henske E P, Jozwiak S, Kingswood J C, Sampson J R, Thiele E A. Tuberous sclerosis complex. Nat Rev Dis Primers. 2016; 2:16035.
4 Lam H C, Siroky B J, Henske E P. Renal disease in tuberous sclerosis complex: pathogenesis and therapy. Nat Rev Nephrol. 2018; 14 (11): 704-716.
5. Johnson S R, Taveira-DaSilva A M, Moss J. Lymphangioleiomyomatosis. Clin Chest Med. 2016; 37 (3): 389-403.
6. Bissler J J, McCormack F X, Young L R, et al. Sirolimus for angiomyolipoma in tuberous sclerosis complex or lymphangioleiomyomatosis. N Engl J Med. 2008; 358 (2): 140-151.
7. McCormack F X, Inoue Y, Moss J, et al. Efficacy and safety of sirolimus in lymphangioleiomyomatosis. N Engl J Med. 2011; 364 (17): 1595-1606.
8 Martin K R, Zhou W, Bowman M J, et al. The genomic landscape of tuberous sclerosis complex. Nat Commun. 2017; 8:15816.
9 Zarei M, Du H, Nassar A H, et al. Tumors with TSC mutations are sensitive to CDK7 inhibition through NRF2 and glutathione depletion. J Exp Med. 2019; 216 (11): 2635-2652.
10. Guo M, Yu J J, Perl A K, et al. Single-Cell Transcriptomic Analysis Identifies a Unique Pulmonary Lymphangioleiomyomatosis Cell. Am J Respir Crit Care Med. 2020; 202 (10): 1373-1387.
11. Obraztsova K, Basil M C, Rue R, et al. mTORC1 activation in lung mesenchyme drives sex- and age-dependent pulmonary structure and function decline. Nat Commun. 2020; 11 (1): 5640.
12. Das P K, Pillai S, Rakib M A, et al. Plasticity of Cancer Stem Cell: Origin and Role in Disease Progression and Therapy Resistance. Stem Cell Rev Rep. 2020; 16 (2): 397-412.
13. Kwiatkowski D J, Thiele E A, Whittemore V H. Tuberous Sclerosis Complex: Genes, Clinical Features and Therapeutics. 1. Aufl. ed. Hoboken: Wiley-Blackwell John Wiley & Sons, Incorporated; 2010.
14. Fluegen G, Avivar-Valderas A, Wang Y, et al. Phenotypic heterogeneity of disseminated tumour cells is preset by primary tumour hypoxic microenvironments. Nat Cell Biol. 2017; 19 (2): 120-132.
15. Tang Y, Kwiatkowski D J, Henske E P. mTORC1 hyperactivation in lymphangioleiomyomatosis leads to ACE2 upregulation in type II pneumocytes: implications for COVID-19. Eur Respir J. 2021; 57 (2).
16. Osterburg A R, Nelson R L, Yaniv B Z, et al. NK cell activating receptor ligand expression in lymphangioleiomyomatosis is associated with lung function decline. JCI Insight. 2016; 1 (16): e87270.
17. Liu H J, Lizotte P H, Du H, et al. TSC2-deficient tumors have evidence of T cell exhaustion and respond to anti-PD-1/anti-CTLA-4 immunotherapy. JCI Insight. 2018; 3 (8).
18. Maisel K, Merrilees M J, Atochina-Vasserman E N, et al. Immune Checkpoint Ligand PD-L1 Is Upregulated in Pulmonary Lymphangioleiomyomatosis. Am J Respir Cell Mol Biol. 2018; 59 (6): 723-732.
19. Giannikou K, Malinowska I A, Pugh T J, et al. Whole Exome Sequencing Identifies TSC1/TSC2 Biallelic Loss as the Primary and Sufficient Driver Event for Renal Angiomyolipoma Development. PLoS Genet. 2016; 12 (8): e1006242.
20. Eble J N, Amin M B, Young R H. Epithelioid angiomyolipoma of the kidney: a report of five cases with a prominent and diagnostically confusing epithelioid smooth muscle component. Am J Surg Pathol. 1997; 21 (10): 1123-1130.
21. Tochio H, Tamaki E, Imai Y, et al. CD68-Positive Cells in Hepatic Angiomyolipoma. Oncology. 2017; 92 Suppl 1:35-39.
22. Li S, Takeuchi F, Wang J A, et al. MCP-1 overexpressed in tuberous sclerosis lesions acts as a paracrine factor for tumor development. J Exp Med. 2005; 202 (5): 617-624.
23. Aran D, Looney A P, Liu L, et al. Reference-based analysis of lung single-cell sequencing reveals a transitional profibrotic macrophage. Nat Immunol. 2019; 20 (2): 163-172.
24. Henske E P, McCormack F X. Lymphangioleiomyomatosis—a wolf in sheep's clothing. J Clin Invest. 2012; 122 (11): 3807-3816.
25. Chilosi M, Pea M, Martignoni G, et al. Cathepsin-k expression in pulmonary lymphangioleiomyomatosis. Mod Pathol. 2009; 22 (2): 161-166.
26. Matsumoto Y, Horiba K, Usuki J, Chu S C, Ferrans V J, Moss J. Markers of cell proliferation and expression of melanosomal antigen in lymphangioleiomyomatosis. Am J Respir Cell Mol Biol. 1999; 21 (3): 327-336.
27. Kumasaka T, Seyama K, Mitani K, et al. Lymphangiogenesis in lymphangioleiomyomatosis: its implication in the progression of lymphangioleiomyomatosis. Am J Surg Pathol. 2004; 28 (8): 1007-1016.
28. Young L R, Inoue Y, McCormack F X. Diagnostic potential of serum VEGF-D for lymphangioleiomyomatosis. N Engl J Med. 2008; 358 (2): 199-200.
29. Martignoni G, Pea M, Reghellin D, et al. Molecular pathology of lymphangioleiomyomatosis and other perivascular epithelioid cell tumors. Arch Pathol Lab Med. 2010; 134 (1): 33-40.
30. Fetsch P A, Fetsch J F, Marincola F M, Travis W, Batts K P, Abati A. Comparison of melanoma antigen recognized by T cells (MART-1) to HMB-45: additional evidence to support a common lineage for angiomyolipoma, lymphangiomyomatosis, and clear cell sugar tumor. Mod Pathol. 1998; 11 (8): 699-703.
31. Rupp C, Scherzer M, Rudisch A, et al. IGFBP7, a novel tumor stroma marker, with growth-promoting effects in colon cancer through a paracrine tumor-stroma interaction. Oncogene. 2015; 34 (7): 815-825.
32. Park C K, Jung W H, Koo J S. Expression of cancer-associated fibroblast-related proteins differs between invasive lobular carcinoma and invasive ductal carcinoma. Breast Cancer Res Treat. 2016; 159 (1): 55-69.
33. Sahai E, Astsaturov I, Cukierman E, et al. A framework for advancing our understanding of cancer-associated fibroblasts. Nature Reviews Cancer. 2020; 20 (3): 174-186.
34. Stuart T, Butler A, Hoffman P, et al. Comprehensive Integration of Single-Cell Data. Cell. 2019; 177 (7): 1888-1902 e1821.
35. Butler A, Hoffman P, Smibert P, Papalexi E, Satija R. Integrating single-cell transcriptomic data across different conditions, technologies, and species. Nat Biotechnol. 2018; 36 (5): 411-420.
36. Lam H C, Baglini C V, Lope A L, et al. p62/SQSTM1 Cooperates with Hyperactive mTORC1 to Regulate Glutathione Production, Maintain Mitochondrial Integrity, and Promote Tumorigenesis. Cancer Res. 2017; 77 (12): 3255-3267.
37. Lee P S, Tsang S W, Moses M A, et al. Rapamycin-insensitive upregulation of MMP2 and other genes in tuberous sclerosis complex 2-deficient lymphangioleiomyomatosis-like cells. Am J Respir Cell Mol Biol. 2010; 42 (2): 227-234.
38. Li C, Liu X, Liu Y, et al. Tuberin Regulates Prostaglandin Receptor-Mediated Viability, via Rheb, in mTORC1-Hyperactive Cells. Mol Cancer Res. 2017; 15 (10): 1318-1330.
39. Hanzelmann S, Castelo R, Guinney J. GSVA: gene set variation analysis for microarray and RNA-seq data. BMC Bioinformatics. 2013; 14:7.
40. Porstmann T, Santos C R, Griffiths B, et al. SREBP activity is regulated by mTORC1 and contributes to Akt-dependent cell growth. Cell Metab. 2008; 8 (3): 224-236.
41. Priolo C, Ricoult S J, Khabibullin D, et al. Tuberous sclerosis complex 2 loss increases lysophosphatidylcholine synthesis in lymphangioleiomyomatosis. Am J Respir Cell Mol Biol. 2015; 53 (1): 33-41.
42. Csibi A, Blenis J. Appetite for destruction: the inhibition of glycolysis as a therapy for tuberous sclerosis complex-related tumors. BMC Biol. 2011; 9:69.
43. Zhang H H, Huang J, Duvel K, et al. Insulin stimulates adipogenesis through the Akt-T SC2-mTORC1 pathway. PLoS One. 2009; 4 (7): e6189.
44. Jones A T, Narov K, Yang J, Sampson J R, Shen M H. Efficacy of Dual Inhibition of Glycolysis and Glutaminolysis for Therapy of Renal Lesions in Tsc2 (+/−) Mice. Neoplasia. 2019; 21 (2): 230-238.
45. Aibar S, Gonzalez-Blas C B, Moerman T, et al. SCENIC: single-cell regulatory network inference and clustering. Nat Methods. 2017; 14 (11): 1083-1086.
46. Makhlouf H R, Ishak K G, Shekar R, Sesterhenn I A, Young D Y, Fanburg-Smith J C. Melanoma markers in angiomyolipoma of the liver and kidney: a comparative study. Arch Pathol Lab Med. 2002; 126 (1): 49-55.
47. Villari D, Grosso M, Vitarelli E, Tuccari G, Barresi G. Melanoma markers in angiomyolipoma. Arch Pathol Lab Med. 2002; 126 (10): 1157.
48. Alesi N, Akl E W, Khabibullin D, et al. TSC2 regulates lysosome biogenesis via a non-canonical RAGC and TFEB-dependent mechanism. Nat Commun. 2021; 12 (1): 4245.
49. Kim K, Pyo S, Um S H. S6 kinase 2 deficiency enhances ketone body production and increases peroxisome proliferator-activated receptor alpha activity in the liver. Hepatology. 2012; 55 (6): 1727-1737.
50. Siroky B J, Yin H, Dixon B P, et al. Evidence for pericyte origin of TSC-associated renal angiomyolipomas and implications for angiotensin receptor inhibition therapy. Am J Physiol Renal Physiol. 2014; 307 (5): F560-570.
51. Luo J, Wang X, Xia Z, et al. Transcriptional factor specificity protein 1 (SP1) promotes the proliferation of glioma cells by up-regulating midkine (MDK). Mol Biol Cell. 2015; 26 (3): 430-439.
52. Reynolds P R, Mucenski M L, Le Cras T D, Nichols W C, Whitsett J A. Midkine is regulated by hypoxia and causes pulmonary vascular remodeling. J Biol Chem. 2004; 279 (35): 37124-37132.
53. Filippou P S, Karagiannis G S, Constantinidou A. Midkine (MDK) growth factor: a key player in cancer progression and a promising therapeutic target. Oncogene. 2019.
54. Choudhuri R, Zhang H T, Donnini S, Ziche M, Bicknell R. An angiogenic role for the neurokines midkine and pleiotrophin in tumorigenesis. Cancer Res. 1997; 57 (9): 1814-1819.
55. Olmeda D, Cerezo-Wallis D, Riveiro-Falkenbach E, et al. Whole-body imaging of lymphovascular niches identifies pre-metastatic roles of midkine. Nature. 2017; 546 (7660): 676-680.
56. Wirka R C, Wagh D, Paik D T, et al. Atheroprotective roles of smooth muscle cell phenotypic modulation and the TCF21 disease gene as revealed by single-cell analysis. Nat Med. 2019; 25 (8): 1280-1289.
57. Yuan H, Yan M, Zhang G, et al. CancerSEA: a cancer single-cell state atlas. Nucleic Acids Res. 2019; 47 (D1): D900-D908.
58. Alvarez-Vasquez F, Sims K J, Cowart L A, Okamoto Y, Voit E O, Hannun Y A. Simulation and validation of modelled sphingolipid metabolism in Saccharomyces cerevisiae. Nature. 2005; 433 (7024): 425-430.
59. Curto R, Voit E O, Sorribas A, Cascante M. Mathematical models of purine metabolism in man. Math Biosci. 1998; 151 (1): 1-49.
60. Tang Y, Gupta A, Garimalla S, et al. Metabolic modeling helps interpret transcriptomic changes during malaria. Biochim Biophys Acta Mol Basis Dis. 2018; 1864 (6 Pt B): 2329-2340.
61. Emmanuel N, Ragunathan S, Shan Q, et al. Purine Nucleotide Availability Regulates mTORC1 Activity through the Rheb GTPase. Cell Rep. 2017; 19 (13): 2665-2680.
62. Hoxhaj G, Hughes-Hallett J, Timson R C, et al. The mTORC1 Signaling Network Senses Changes in Cellular Purine Nucleotide Levels. Cell Rep. 2017; 21 (5): 1331-1346.
63. Tang Y, El-Chemaly S, Taveira-Dasilva A, et al. Alterations in Polyamine Metabolism in Patients With Lymphangioleiomyomatosis and Tuberous Sclerosis Complex 2-Deficient Cells. Chest. 2019; 156 (6): 1137-1148.
64. Nishimura T, Nakata A, Chen X, et al. Cancer stem-like properties and gefitinib resistance are dependent on purine synthetic metabolism mediated by the mitochondrial enzyme MTHFD2. Oncogene. 2019; 38 (14): 2464-2481.
65. Prunier C, Baker D, Ten Dijke P, Ritsma L. TGF-beta Family Signaling Pathways in Cellular Dormancy. Trends Cancer. 2019; 5 (1): 66-78.
66. Phan T G, Croucher P I. The dormant cancer cell life cycle. Nat Rev Cancer. 2020; 20 (7): 398-411.
67. Borgen E, Rypdal M C, Sosa M S, et al. NR2F1 stratifies dormant disseminated tumor cells in breast cancer patients. Breast Cancer Res. 2018; 20 (1): 120.
68. Yu J J, Robb V A, Morrison T A, et al. Estrogen promotes the survival and pulmonary metastasis of tuberin-null cells. Proc Natl Acad Sci USA. 2009; 106 (8): 2635-2640.
69. Yu J, Astrinidis A, Howard S, Henske E P. Estradiol and tamoxifen stimulate LAM-associated angiomyolipoma cell growth and activate both genomic and nongenomic signaling pathways. Am J Physiol Lung Cell Mol Physiol. 2004; 286 (4): L694-700.
70. Carsillo T, Astrinidis A, Henske E P. Mutations in the tuberous sclerosis complex gene TSC2 are a cause of sporadic pulmonary lymphangioleiomyomatosis. Proc Natl Acad Sci USA. 2000; 97 (11): 6085-6090.
71. Glasgow C G, Avila N A, Lin J P, Stylianou M P, Moss J. Serum vascular endothelial growth factor-D levels in patients with lymphangioleiomyomatosis reflect lymphatic involvement. Chest. 2009; 135 (5): 1293-1300.
72. Gustavsson H, Jennbacken K, Welen K, Damber J E. Altered expression of genes regulating angiogenesis in experimental androgen-independent prostate cancer. Prostate. 2008; 68 (2): 161-170.
73. De Angelis M L, Francescangeli F, La Torre F, Zeuner A. Stem Cell Plasticity and Dormancy in the Development of Cancer Therapy Resistance. Frontiers in Oncology. 2019; 9 (626).
74. Visvader J E, Lindeman G J. Cancer stem cells in solid tumours: accumulating evidence and unresolved questions. Nat Rev Cancer. 2008; 8 (10): 755-768.
75. Dean M, Fojo T, Bates S. Tumour stem cells and drug resistance. Nat Rev Cancer. 2005; 5 (4): 275-284.
76. Aguirre-Ghiso J A. Models, mechanisms and clinical evidence for cancer dormancy. Nat Rev Cancer. 2007; 7 (11): 834-846.
77. Mirkin B L, Clark S, Zheng X, et al. Identification of midkine as a mediator for intercellular transfer of drug resistance. Oncogene. 2005; 24 (31): 4965-4974.
78. Goncharova E A, Goncharov D A, Fehrenbach M, et al. Prevention of alveolar destruction and airspace enlargement in a mouse model of pulmonary lymphangioleiomyomatosis (LAM). Sci Transl Med. 2012; 4 (154): 154ra134.
79. Hao H, Maeda Y, Fukazawa T, et al. Inhibition of the growth factor MDK/midkine by a novel small molecule compound to treat non-small cell lung cancer. PLoS One. 2013; 8 (8): e71093.
80. Cerezo-Wallis D, Contreras-Alcalde M, Troule K, et al. Midkine rewires the melanoma microenvironment toward a tolerogenic and immune-resistant state. Nat Med. 2020; 26 (12): 1865-1877.
81. Cokol M, Chua H N, Tasan M, et al. Systematic exploration of synergistic drug pairs. Mol Syst Biol. 2011; 7:544.
82. Kalan L, Wright G D. Antibiotic adjuvants: multicomponent anti-infective strategies. Expert Rev Mol Med. 2011; 13: e5.
83. Miranda A, Hamilton P T, Zhang A W, et al. Cancer stemness, intratumoral heterogeneity, and immune response across cancers. Proc Natl Acad Sci USA. 2019; 116 (18): 9020-9029.
84. Foulds K E, Zenewicz L A, Shedlock D J, Jiang J, Troy A E, Shen H. Cutting Edge: CD4 and CD8 T Cells Are Intrinsically Different in Their Proliferative Responses. The Journal of Immunology. 2002; 168 (4): 1528-1532.
85. Bergen V, Lange M, Peidli S, Wolf F A, Theis F J. Generalizing RNA velocity to transient cell states through dynamical modeling. Nat Biotechnol. 2020; 38 (12): 1408-1414.
86. Kumar M P, Du J, Lagoudas G, et al. Analysis of Single-Cell RNA-Seq Identifies Cell-Cell Communication Associated with Tumor Characteristics. Cell Rep. 2018; 25 (6): 1458-1468 e1454.
87. Lu W, Yu W, He J, et al. Reprogramming immunosuppressive myeloid cells facilitates immunotherapy for colorectal cancer. EMBO Mol Med. 2020: e12798. 88. Mahoney K M, Freeman G J. Acidity changes immunology: a new VISTA pathway. Nat Immunol. 2020; 21 (1): 13-16.
89. Das M, Zhu C, Kuchroo V K. Tim-3 and its role in regulating anti-tumor immunity. Immunol Rev. 2017; 276 (1): 97-111.
90. Tirosh I, Izar B, Prakadan S M, et al. Dissecting the multicellular ecosystem of metastatic melanoma by single-cell RNA-seq. Science. 2016; 352 (6282): 189-196.
91. Starossom S C, Mascanfroni I D, Imitola J, et al. Galectin-1 deactivates classically activated microglia and protects from inflammation-induced neurodegeneration. Immunity. 2012; 37 (2): 249-263.
92. Baitsch D, Bock H H, Engel T, et al. Apolipoprotein E induces antiinflammatory phenotype in macrophages. Arterioscler Thromb Vasc Biol. 2011; 31 (5): 1160-1168.
93. Zhao Y, Wu X, Li X, et al. TREM2 Is a Receptor for beta-Amyloid that Mediates Microglial Function. Neuron. 2018; 97 (5): 1023-1031 e1027.
94. Peng Q, Malhotra S, Torchia J A, Kerr W G, Coggeshall K M, Humphrey M B. TREM2- and DAP12-dependent activation of PI3K requires DAP10 and is inhibited by SHIP1. Sci Signal. 2010; 3 (122): ra38.
95. Ulland T K, Song W M, Huang S C, et al. TREM2 Maintains Microglial Metabolic Fitness in Alzheimer's Disease. Cell. 2017; 170 (4): 649-663 e613.
96. Molgora M, Esaulova E, Vermi W, et al. TREM2 Modulation Remodels the Tumor Myeloid Landscape Enhancing Anti-PD-1 Immunotherapy. Cell. 2020; 182 (4): 886-900 e817.
97. Katzenelenbogen Y, Sheban F, Yalin A, et al. Coupled scRNA-Seq and Intracellular Protein Activity Reveal an Immunosuppressive Role of TREM2 in Cancer. Cell. 2020; 182 (4): 872-885 e819.
98. Leung G A, Cool T, Valencia C H, Worthington A, Beaudin A E, Forsberg E C. The lymphoid-associated interleukin 7 receptor (IL7R) regulates tissue-resident macrophage development. Development. 2019; 146 (14).
99. Yang M, Mckay D, Pollard J W, Lewis C E. Diverse Functions of Macrophages in Different Tumor Microenvironments. Cancer Res. 2018; 78 (19): 5492-5503.
100. Zhou L, Zhuo H, Ouyang H, et al. Glycoprotein non-metastatic melanoma protein b (Gpnmb) is highly expressed in macrophages of acute injured kidney and promotes M2 macrophages polarization. Cell Immunol. 2017; 316:53-60.
101. Sarvaria A, Madrigal J A, Saudemont A. B cell regulation in cancer and anti-tumor immunity. Cell Mol Immunol. 2017; 14 (8): 662-674.
102. Patel J, Bozeman E N, Selvaraj P. Taming dendritic cells with TIM-3: another immunosuppressive strategy used by tumors. Immunotherapy. 2012; 4 (12): 1795-1798.
103. de Mingo Pulido A, Gardner A, Hiebler S, et al. TIM-3 Regulates CD103 (+) Dendritic Cell Function and Response to Chemotherapy in Breast Cancer. Cancer Cell. 2018; 33 (1): 60-74 e66.
104. Hanahan D, Weinberg R A. Hallmarks of cancer: the next generation. Cell. 2011; 144 (5): 646-674.
105. Menon S, Manning B D. Common corruption of the mTOR signaling network in human tumors. Oncogene. 2008; 27 Suppl 2 (0 2): S43-51.
106. Alayev A, Holz M K. mTOR signaling for biological control and cancer. J Cell Physiol. 2013; 228 (8): 1658-1664.
107. Guertin D A, Sabatini D M. Defining the role of mTOR in cancer. Cancer Cell. 2007; 12 (1): 9-22.
108. Kang H C, Kim I J, Park J H, et al. Identification of genes with differential expression in acquired drug-resistant gastric cancer cells using high-density oligonucleotide microarrays. Clin Cancer Res. 2004; 10 (1 Pt 1): 272-284.
109. Bonsib S M, Moghadamfalahi M, Bhalodia A. Lymphatic differentiation in renal angiomyolipomas. Hum Pathol. 2009; 40 (3): 374-380.
110. Young L R, Vandyke R, Gulleman P M, et al. Serum vascular endothelial growth factor-D prospectively distinguishes lymphangioleiomyomatosis from other diseases. Chest. 2010; 138 (3): 674-681.
111. McLane L M, Abdel-Hakeem M S, Wherry E J. CD8 T Cell Exhaustion During Chronic Viral Infection and Cancer. Annu Rev Immunol. 2019; 37:457-495.
112. DeNardo D G, Ruffell B. Macrophages as regulators of tumour immunity and immunotherapy. Nat Rev Immunol. 2019; 19 (6): 369-382.
113. Yeh F L, Wang Y, Tom I, Gonzalez L C, Sheng M. TREM2 Binds to Apolipoproteins, Including APOE and CLU/APOJ, and Thereby Facilitates Uptake of Amyloid-Beta by Microglia. Neuron. 2016; 91 (2): 328-340.
114. De Cicco P, Ercolano G, Ianaro A. The New Era of Cancer Immunotherapy: Targeting Myeloid-Derived Suppressor Cells to Overcome Immune Evasion. Frontiers in immunology. 2020; 11:1680-1680.
115. La Manno G, Soldatov R, Zeisel A, et al. RNA velocity of single cells. Nature. 2018; 560 (7719): 494-498.
116. Liberzon A, Birger C, Thorvaldsdottir H, Ghandi M, Mesirov J P, Tamayo P. The Molecular Signatures Database (MSigDB) hallmark gene set collection. Cell Syst. 2015; 1 (6): 417-425.
117. Ramilowski J A, Goldberg T, Harshbarger J, et al. A draft network of ligand-receptor-mediated multicellular signalling in human. Nat Commun. 2015; 6:7866.
118. Zou, Z., Tao, T., Li, H. et al. mTOR signaling pathway and mTOR inhibitors in cancer: progress and challenges. Cell Biosci 10, 31 (2020).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a Tuberous Sclerosis Complex (TSC)-associated disease, the method comprising administering to a subject in need thereof a therapeutically effective amount of a therapeutic agent targeting mTORC1 and a therapeutic agent targeting MDK.

2. The method of claim 1, wherein the TSC-associated disease is angiomyolipoma (AML) or lymphangioleiomyomatosis (LAM).

3. The method of claim 1, wherein the TSC-associated disease is a cortical dysplasia, subependymal nodule, subependymal giant cell astrocytoma (SEGA), cardiac rhabdomyoma, dermatologic or ophthalmic tumor, renal cyst, multifocal micronodular pneumocyte hyperplasia (MMPH), splenic hamartoma, or perivascular epithelioid tumor (PEComa).

4. A method for treating lymphangioleiomyomatosis (LAM) or angiomyolipoma (AML), the method comprising administering to a subject in need thereof a combination of a therapeutic agent targeting mTORC1 and a therapeutic agent targeting MDK.

5. The method of claim 4, wherein the subject does not have a diagnosis of TSC or a mutation in the TSC1 or TSC2 tumor suppressor genes.

6. The method of any of claims 1-5, wherein the mTORC1 inhibitor is selected from the group consisting of MLN0128, MHY1485, PI-103, PP242 (torkinib), PP30, XL388, AZD2014 (vistusertib), voxtalisib (SAR24540; XL765), vistusertib, OSI-227, WAY-600, WYE-132, WYE-687, or sapanisertib (TAK-228); PF-04691502; Gedatolisib (PKI-587; PF05212384); AZD 8055 ((5-(2,4-bis((S)-3-methylmorpholino)pyrido[2,3-d]pyrimidin-7-yl)-2-methoxyphenyl) methanol); Torin-1 (1-[4-[4-(1-oxopropyl)-1-piperazinyl]-3-(trifluoromethyl)phenyl]-9-(3-quinolinyl)-benzo[h]-1,6-naphthyridin-2 (1H)-one); torin-2; apitolisib; gedatolisib; GSK2126458 (GSK458); CC-223; 4H-1-benzopyran-4-one derivatives; rapamycin (sirolimus) and derivatives thereof, including: temsirolimus, umirolimus, everolimus, ridaforolimus (deforolimus), and zotarolimus; rapalogs, optionally AP23464, AP23841, 40-(2-hydroxyethyl) rapamycin; 40-[3-hydroxy (hydroxymethyl)methylpropanoate]-rapamycin (CC1779); 40-epi-(tetrazolyt)-rapamycin (ABT578); 32-deoxorapamycin; 16-pentynyloxy-32 (S)-dihydrorapanycin; and phosphorus-containing rapamycin derivatives; cornarin A, dactolisib, omipalisib, samotolisib, KU-0063794, gadatolisib, dactosulib tosylate, CC-115, apitolisib, bimarilisib, VS-5584, GDC-0349, CZ415, WYE-354, onatasertib, mTOR-inhibitor 3, palomid 529, PQR620, (+)-usnic acid, MT 63-78, MTI-31, FT-1518, AZD3147, and RMC-5552.

7. The method of any of claims 1-6, wherein the MDK inhibitor is iMDK; an anti-midkine antibody; an RNA Aptamer; or an inhibitory nucleic acid targeting midkine.

8. The method of claim 7, wherein the inhibitory nucleic acid targeting midkine is an antisense oligonucleotide, siRNA, or shRNA.

9. The method of claims 1-8 further comprising administering a checkpoint inhibitor or a treatment comprising chemotherapy, radiotherapy, and/or resection.

10. A composition comprising a combination of a therapeutic agent targeting mTORC1 and a therapeutic agent targeting MDK, and optionally a checkpoint inhibitor.

11. The composition of claim 10, wherein the mTORC1 inhibitor is selected from the group consisting of MLN0128, MHY1485, PI-103, PP242 (torkinib), PP30, XL388, AZD2014 (vistusertib), voxtalisib (SAR24540; XL765), vistusertib, OSI-227, WAY-600, WYE-132, WYE-687, or sapanisertib (TAK-228); PF-04691502; Gedatolisib (PKI-587; PF05212384); AZD 8055 ((5-(2,4-bis((S)-3-methylmorpholino)pyrido[2,3-d]pyrimidin-7-yl)-2-methoxyphenyl) methanol); Torin-1 (1-[4-[4-(1-oxopropyl)-1-piperazinyl]-3-(trifluoromethyl)phenyl]-9-(3-quinolinyl)-benzo[h]-1,6-naphthyridin-2 (1H)-one); torin-2; apitolisib; gedatolisib; GSK2126458 (GSK458); CC-223; 4H-1-benzopyran-4-one derivatives; rapamycin (sirolimus) and derivatives thereof, including: temsirolimus, umirolimus, everolimus, ridaforolimus (deforolimus), and zotarolimus; rapalogs, optionally AP23464, AP23841, 40-(2-hydroxyethyl) rapamycin; 40-[3-hydroxy (hydroxymethyl)methylpropanoate]-rapamycin (CC1779); 40-epi-(tetrazolyt)-rapamycin (ABT578); 32-deoxorapamycin; 16-pentynyloxy-32 (S)-dihydrorapanycin; and phosphorus-containing rapamycin derivatives; cornarin A, dactolisib, omipalisib, samotolisib, KU-0063794, gadatolisib, dactosulib tosylate, CC-115, apitolisib, bimarilisib, VS-5584, GDC-0349, CZ415, WYE-354, onatasertib, mTOR-inhibitor 3, palomid 529, PQR620, (+)-usnic acid, MT 63-78, MTI-31, FT-1518, AZD3147, and RMC-5552.

12. The composition of any of claims 10-11, wherein the MDK inhibitor is iMDK; an anti-midkine antibody; an RNA Aptamer; or an inhibitory nucleic acid targeting midkine.

13. The composition of claim 12, wherein the inhibitory nucleic acid targeting midkine is an antisense oligonucleotide, siRNA, or shRNA.

14. A therapeutic agent targeting mTORC1 and a therapeutic agent targeting MDK for use in a method of treating a Tuberous Sclerosis Complex (TSC)-associated disease.

15. A therapeutic agent targeting mTORC1 and a therapeutic agent targeting MDK for the use of claim 14, wherein the TSC-associated disease is angiomyolipoma (AML) or lymphangioleiomyomatosis (LAM).

16. The therapeutic agent targeting mTORC1 and a therapeutic agent targeting MDK for the use of claim 14, wherein the TSC-associated disease is a cortical dysplasia, subependymal nodule, subependymal giant cell astrocytoma (SEGA), cardiac rhabdomyoma, dermatologic or ophthalmic tumor, renal cyst, multifocal micronodular pneumocyte hyperplasia (MMPH), splenic hamartoma, or perivascular epithelioid tumor (PEComa).

17. A therapeutic agent targeting mTORC1 and a therapeutic agent targeting MDK for use in a method of treating lymphangioleiomyomatosis (LAM) or angiomyolipoma (AML).

18. The therapeutic agent targeting mTORC1 and a therapeutic agent targeting MDK for the use of claim 17, wherein the subject does not have a diagnosis of TSC or a mutation in the TSC1 or TSC2 tumor suppressor genes.

19. The therapeutic agent targeting mTORC1 and a therapeutic agent targeting MDK for the use of any of claims 14-18, wherein the mTORC1 inhibitor is selected from the group consisting of MLN0128, MHY1485, PI-103, PP242 (torkinib), PP30, XL388, AZD2014 (vistusertib), voxtalisib (SAR24540; XL765), vistusertib, OSI-227, WAY-600, WYE-132, WYE-687, or sapanisertib (TAK-228); PF-04691502; Gedatolisib (PKI-587; PF05212384); AZD 8055 ((5-(2,4-bis((S)-3-methylmorpholino)pyrido[2,3-d]pyrimidin-7-yl)-2-methoxyphenyl) methanol); Torin-1 (1-[4-[4-(1-oxopropyl)-1-piperazinyl]-3-(trifluoromethyl)phenyl]-9-(3-quinolinyl)-benzo[h]-1,6-naphthyridin-2 (1H)-one); torin-2; apitolisib; gedatolisib; GSK2126458 (GSK458); CC-223; 4H-1-benzopyran-4-one derivatives; rapamycin (sirolimus) and derivatives thereof, including: temsirolimus, umirolimus, everolimus, ridaforolimus (deforolimus), and zotarolimus; rapalogs, optionally AP23464, AP23841, 40-(2-hydroxyethyl) rapamycin; 40-[3-hydroxy (hydroxymethyl)methylpropanoate]-rapamycin (CC1779); 40-epi-(tetrazolyt)-rapamycin (ABT578); 32-deoxorapamycin; 16-pentynyloxy-32 (S)-dihydrorapanycin; and phosphorus-containing rapamycin derivatives; cornarin A, dactolisib, omipalisib, samotolisib, KU-0063794, gadatolisib, dactosulib tosylate, CC-115, apitolisib, bimarilisib, VS-5584, GDC-0349, CZ415, WYE-354, onatasertib, mTOR-inhibitor 3, palomid 529, PQR620, (+)-usnic acid, MT 63-78, MTI-31, FT-1518, AZD3147, and RMC-5552.

20. The therapeutic agent targeting mTORC1 and a therapeutic agent targeting MDK for the use of any of claims 14-19, wherein the MDK inhibitor is iMDK; an anti-midkine antibody; an RNA Aptamer; or an inhibitory nucleic acid targeting midkine.

21. The therapeutic agent targeting mTORC1 and a therapeutic agent targeting MDK for the use of claim 20, wherein the inhibitory nucleic acid targeting midkine is an antisense oligonucleotide, siRNA, or shRNA.

22. The therapeutic agent targeting mTORC1 and a therapeutic agent targeting MDK for the use of any of claims 14-21, wherein the method further comprises administering a checkpoint inhibitor or a treatment comprising chemotherapy, radiotherapy, and/or resection.

23. A method for selecting a treatment for a Tuberous Sclerosis Complex (TSC)-associated disease in a subject, the method comprising:
determining a level of MDK in a sample from the subject;
comparing the level of MDK in the sample to a reference level of MDK;
identifying a subject who has a level of MDK above the reference level; and
selecting a treatment comprising administering to identified the subject a therapeutically effective amount of a therapeutic agent targeting mTORC1 and a checkpoint inhibitor, and optionally a therapeutic agent targeting MDK.

24. The method of claim 23, wherein the TSC-associated disease is angiomyolipoma (AML) or lymphangioleiomyomatosis (LAM).

25. The method of claim 23, wherein the TSC-associated disease is a cortical dysplasia, subependymal nodule, subependymal giant cell astrocytoma (SEGA), cardiac rhabdomyoma, dermatologic or ophthalmic tumor, renal cyst, multifocal micronodular pneumocyte hyperplasia (MMPH), splenic hamartoma, or perivascular epithelioid tumor (PEComa).

26. A method for selecting a treatment for lymphangioleiomyomatosis (LAM) or angiomyolipoma (AML), the method comprising:
determining a level of MDK in a sample from the subject;
comparing the level of MDK in the sample to a reference level of MDK;
identifying a subject who has a level of MDK above the reference level; and
selecting a treatment comprising administering to identified the subject a therapeutically effective amount of a therapeutic agent targeting mTORC1 and a checkpoint inhibitor, and optionally a therapeutic agent targeting MDK.

27. The method of claim 26, wherein the subject does not have a diagnosis of TSC or a mutation in the TSC1 or TSC2 tumor suppressor genes.

28. The method of any of claims 23-27, wherein the mTORC1 inhibitor is selected from the group consisting of MLN0128, MHY1485, PI-103, PP242 (torkinib), PP30, XL388, AZD2014 (vistusertib), voxtalisib (SAR24540; XL765), vistusertib, OSI-227, WAY-600, WYE-132, WYE-687, or sapanisertib (TAK-228); PF-04691502; Gedatolisib (PKI-587; PF05212384); AZD 8055 ((5-(2,4-bis((S)-3-methylmorpholino)pyrido[2,3-d]pyrimidin-7-yl)-2-methoxyphenyl) methanol); Torin-1 (1-[4-[4-(1-oxopropyl)-1-piperazinyl]-3-(trifluoromethyl)phenyl]-9-(3-quinolinyl)-benzo[h]-1,6-naphthyridin-2 (1H)-one); torin-2; apitolisib; gedatolisib; GSK2126458 (GSK458); CC-223; 4H-1-benzopyran-4-one derivatives; rapamycin (sirolimus) and derivatives thereof, including: temsirolimus, umirolimus, everolimus, ridaforolimus (deforolimus), and zotarolimus; rapalogs, optionally AP23464, AP23841, 40-(2-hydroxyethyl) rapamycin; 40-[3-hydroxy (hydroxymethyl)methylpropanoate]-rapamycin (CC1779); 40-epi-(tetrazolyt)-rapamycin (ABT578); 32-deoxorapamycin; 16-pentynyloxy-32 (S)-dihydrorapanycin; and phosphorus-containing rapamycin derivatives; cornarin A, dactolisib, omipalisib, samotolisib, KU-0063794, gadatolisib, dactosulib tosylate, CC-115, apitolisib, bimarilisib, VS-5584, GDC-0349, CZ415, WYE-354, onatasertib, mTOR-inhibitor 3, palomid 529, PQR620, (+)-usnic acid, MT 63-78, MTI-31, FT-1518, AZD3147, and RMC-5552.

29. The method of any of claims 23-28, wherein the MDK inhibitor is iMDK; an anti-midkine antibody; an RNA Aptamer; or an inhibitory nucleic acid targeting midkine

30. The method of claim 29, wherein the inhibitory nucleic acid targeting midkine is an antisense oligonucleotide, siRNA, or shRNA.

31. The method of claims 23-30, further comprising administering the treatment to the identified subject.

32. The method of claim 30, further comprising administering a treatment comprising chemotherapy, radiotherapy, and/or resection.

33. Any of claim 9, 10, 22, 23, or 26, wherein the checkpoint inhibitor is an inhibitor of PD-1 signaling, optionally an antibody that binds to PD-1, CD40, or PD-L1; an inhibitor of Tim3 or Lag3, optionally an antibody that binds to Tim3 or Lag3; an inhibitor of CTLA4, optionally an antibody that binds to CTLA-4; or an inhibitor of T-cell immunoglobulin and ITIM domains (TIGIT), optionally an antibody that binds to TIGIT.
